(19) United States
(12) Patent Application Publication
(10) Pub. No.: US 2025/0221959 A1
(43) Pub. Date: Jul. 10, 2025

(54) COMBINED USE OF INDIVIDUAL COMPOUNDS FOR TREATMENT OF CHRONIC PAIN DISORDERS

(71) Applicant: VasoCeuticals, Inc., San Diego, CA (US)

(72) Inventors: Frank Lambert RICE, Delmar (NY); Phillip James ALBRECHT, Delmar, NY (US); Tobias MOELLER-BERTRAM, San Diego, CA (US)

(73) Assignee: VasoCeuticals, Inc., San Diego, CA (US)

(21) Appl. No.: 18/848,048

(22) PCT Filed: Mar. 16, 2023
(86) PCT No.: PCT/US2023/015421

§ 371 Date: Sep. 17, 2024

Related U.S. Application Data
(60) Provisional application No. 63/320,931, filed on Mar. 17, 2022.

(51) Int. Cl. A61K 31/381; A61K 31/165; A61K 31/4168; A61K 31/4545; A61K 39/395

(52) U.S. Cl. CPC A61K 31/381; A61K 31/165; A61K 31/4168; A61K 31/4545; A61K 39/3955

(57) ABSTRACT

Disclosed herein are pharmaceutical compositions comprising several active ingredients in combination for treatment of chronic pain-related disorders involving a specific neurovascular AVS pathology, while minimizing undesirable side effects. Also provided are methods for treating subjects using such pharmaceutical compositions.

### PRIORITY CLAIM

The present application claims the benefit of U.S. Application No. 63/320,931 filed Mar. 17, 2022, the full disclosure of which is incorporated herein by reference in its entirety for all purposes.

### FIELD OF THE INVENTION

The present disclosure relates to pharmaceutical combinations of several active ingredients for treatment of patients suffering from a variety of painful afflictions that share a combination of medically unexplained symptoms (MUS), typically including chronic widespread pain, fatigue, non-restorative sleep, and cognitive dysfunction (“brain fog”), while minimizing undesirable side effects. The disclosure also relates to methods for treating patients using such pharmaceutical compositions.

### BACKGROUND OF THE INVENTION

Fibromyalgia (FM), post-traumatic stress disorder (PTSD), myalgic encephalomyelitis/chronic fatigue syndrome (ME/CFS), irritable bowel syndrome (IBS), post-viral syndromes (e.g., post-COVID-19 (long COVID)), post-war syndromes (e.g., Post Gulf War), environmentally-implicated syndromes (e.g. extreme chemical sensitivity and sick-building syndrome, and other similar afflictions are characterized by medically unexplained symptoms (MUS) that include chronic widespread pain as the major complaint, along with fatigue, non-restorative sleep, and cognitive dysfunction. Lacking evidence of pathologies in the peripheral nervous system (PNS), disorders characterized by MUS are often attributed to an induced Central Nervous System (CNS) hyperactivity dysfunction referred to as Central Sensitization Syndrome. Disorders with MUS have been characterized based almost entirely on subjective patient perceptions of differences in the proportions of the shared MUS, additional unshared symptoms, and etiology histories. The subjective symptomology and complexity of these types of disorders with MUS makes them notoriously difficult to diagnose and treat, and therefore they are still often dismissed as psychosomatic. Current therapeutics for disorders with MUS remain limited, and those commonly utilized offer inconsistent relief that is often diminished by intolerable side-effects at doses used to achieve efficacy. Thus, there remains a strong need for novel, rationally developed therapeutics that are successful in treating these types of debilitating chronic pain conditions, while avoiding adverse side-effects.

A goal of research to treat acute and chronic pain conditions is to identify the biological mechanisms (e.g., molecular, cellular, and/or structural) that initiate and maintain the neuronal activity producing pain perceptions and to rationally develop therapeutics that will act on those mechanisms to promote pain relief. This goal remains largely unmet for numerous chronic pain conditions, notably because the underlying pathologic mechanisms driving chronic pain perceptions remain unknown.

### SUMMARY OF THE INVENTION

Aspects of the present disclosure are based on the discovery of a significant neurovascular pathology in the hands of FM and PTSD patients (Albrecht et al., 2013, ibid). The functional implications of the discovered peripheral neurovascular pathology provide a novel disease target, and suggest the use of specific combinations of active compounds to modulate the skewed neurovascular functioning and achieve resolution of common MUS, particularly chronic widespread pain, fatigue, non-restorative sleep, and cognitive dysfunction (see FIG. 2 for a schematic of the compounds and their combined corrective resolution).

This disclosure describes pharmaceutical combinations and methods for treating chronic conditions characterized by MUS including diffuse widespread chronic pain, fatigue, non-restorative sleep, and cognitive dysfunction (“brain fog”) (Albrecht and Rice, 2016, Rev Environ Health 31:281-294, PMID: 27105483) (see FIG. 2). In various embodiments, the combined compounds are effective in treating patients with FM, PTSD, ME/CFS, IBS, post-viral syndromes, post-war syndromes, and other afflictions characterized by MUS of chronic widespread pain, fatigue, non-restorative sleep, and cognitive dysfunction. In some embodiments, the pharmaceutical combinations described herein are composed of compounds that are approved for individual use by the United States Federal Drug Administration (FDA). In various embodiments, the combined compounds are efficacious at lower concentrations than when they would be administered alone, due to additive and/or synergistic actions of the combined compounds. Utilizing lower concentrations of these compounds will allow for a greater range of efficacious utility while decreasing side-effects. Aspects of the present disclosure are supported by the scientific evidence for blood flow regulation and peripheral mechanisms of homeostasis.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows a schematic illustration of normal cutaneous microneurovascular organization and function for balancing thermal and metabolic regulation under different thermal and exercise challenges (panels #1-4). Also shown (panel #5) is a schematic illustration of pathological microneurovascular organization in, e.g., subjects with fibromyalgia and/or PTSD, according to certain aspects of this disclosure.

FIG. 2 shows a schematic illustration of the molecular mechanism of AVS neurovascular regulation, AVS pathology in fibromyalgia and PTSD patients, and a multi-modal therapeutic strategy, according to certain aspects of this disclosure.

### DETAILED DESCRIPTION OF THE INVENTION

### I. Introduction

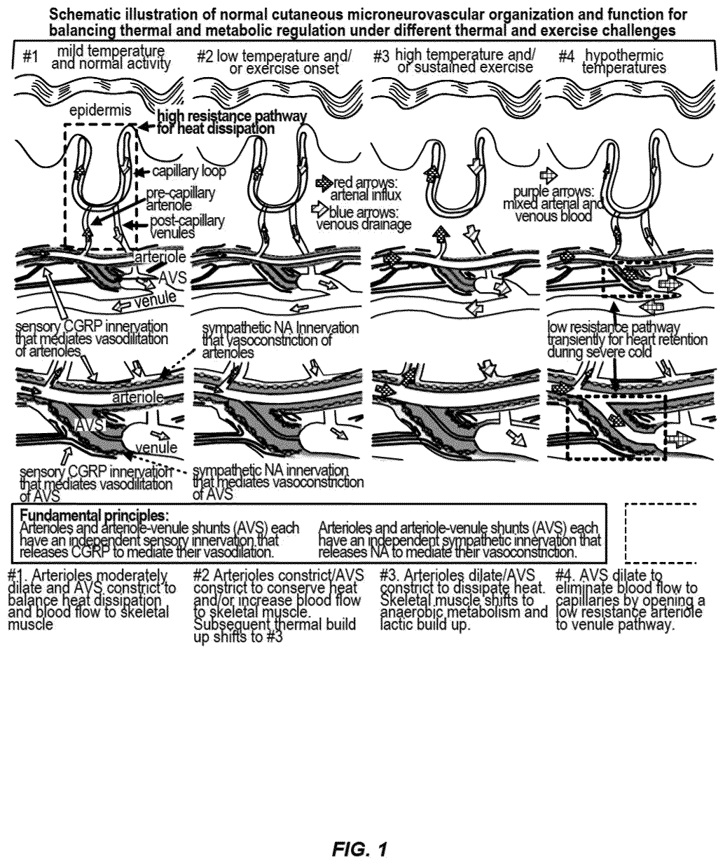

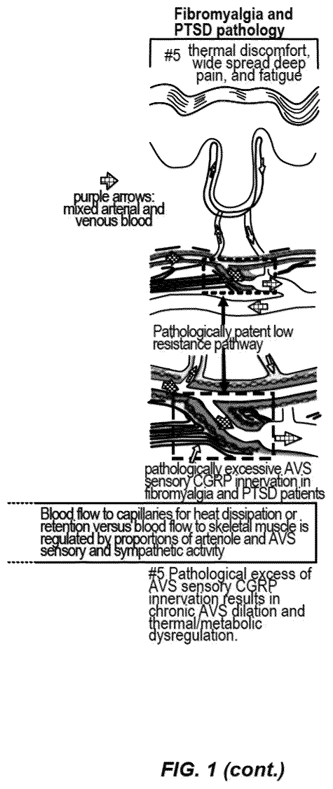

Certain aspects of the present disclosure are based from the discovery and neurochemical profiling of a neurovascular pathology in the hands of FM and PTSD patients (Albrecht et al., 2013, Pain Med. 14:895-915. PMID: 23691965). Using a comprehensive immunolabeling platform for complex tissues, our group identified a unique neurovascular pathology associated with specific regulatory structures in glabrous skin of FM patients (Albrecht et al., 2013, ibid.), and subsequent work also demonstrated a similar pathology in a small cohort of PTSD patients (see FIG. 1 for a schematic of the pathology). The functional implications for this pathology suggest that dysfunctional vascular regulation may be a driving source for the MUS of chronic widespread pain, fatigue, non-restorative sleep, and cognitive dysfunction reported in FM and PTSD, and may also be present in other disorders with similar MUS, for example IBS, post-viral syndromes (e.g., post-COVID-19 (long COVID)), post-war syndromes (e.g., Post Gulf War), and others. The inventors considered the current best-practice treatment methods for these disorders, the novel discovery and neurochemical profiling of the peripheral neurovascular pathology, and the functional implications of the peripheral neurovascular pathology in proposing the unobvious methods and compositions described herein for the use of specific combinations of individual compounds, as described herein.

Fibromyalgia (FM), post-traumatic stress disorder (PTSD), myalgic encephalomyelitis/chronic fatigue syndrome (ME/CFS), irritable bowel syndrome (IBS), post-viral syndromes (e.g., post-COVID-19 (long COVID)), post-war syndromes (e.g., Post-Gulf War), environmentally-implicated syndromes (e.g., chemical hypersensitivity and sick-building syndrome), and other similar disorders, are all characterized by medically unexplained symptoms (MUS). Intractable widespread chronic pain is one of the most debilitating MUS, which also commonly include fatigue, non-restorative sleep, and cognitive dysfunction (brain fog). Treatments for chronic pain disorders remain limited, and the currently utilized therapies lack efficacy and are limited by side-effects. Moreover, development of novel analgesic compounds has been repeatedly hindered by late-stage clinical trial failures. These failures to translate promising basic animal pain research into efficacious therapies in humans has been highlighted recently by several government-funded specialty groups convened to specifically address these issues (see IMMPACT, ACCTION). Importantly, the technology utilized herein to discover the novel neurovascular pathology target—comprehensive skin biopsy immunolabel profiling—was recommended by these groups as a means to improve discovery and development of chronic pain therapeutics. The present disclosure is based on molecular and morphologic properties of a neurovascular PNS pathology which provides a novel unifying functional mechanism for disorders with MUS and suggests novel therapeutic targets and treatment strategies.

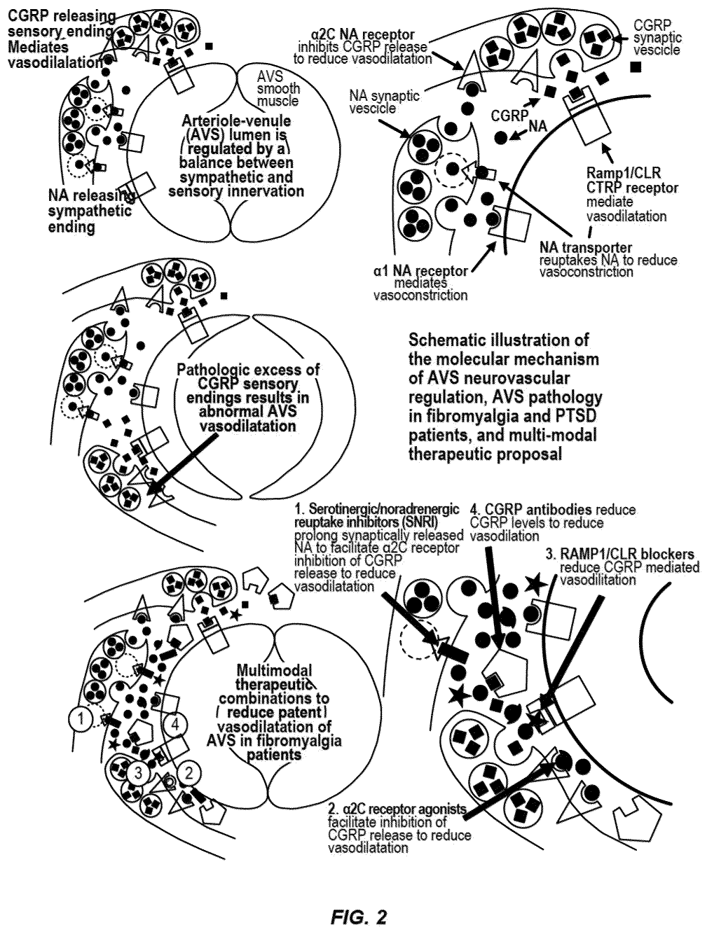

Certain aspects of the current disclosure are based from novel neurovascular pathology evidence suggesting that targeting nervous system mechanisms associated with peripheral vascular tone and blood flow regulation will provide significant benefit to disorders with MUS, particularly chronic widespread pain, fatigue, non-restorative sleep, and cognitive dysfunction as commonly reported in FM, PTSD, ME/CSF, IBS, post-viral syndromes, post-war syndromes, and other disorders (see FIGS. 1 and 2).

Nervous System Function and Neuronal Circuits Overview. The human nervous system is composed of billions of specialized nerve cells (neurons) developmentally organized into distinct groups (termed nuclei and ganglia) which are functionally divided into the Central Nervous System (CNS; brain and spinal cord) and the Peripheral Nervous System (PNS; peripheral nerves), and these systems are fully interconnected to each other. Neurons are specialized electrically-active cells capable of maintaining and directionally distributing electrical current, termed action potentials (AP), along unique cellular structures, called axons, which extend from each neuron cell body to a distant neuron or target. Bundled axon processes, called nerves, interconnect specific neuron groups into complex active circuits to create a highly interconnected neural network of electrochemical signaling. The complex patterned activity across the neural network serves to monitor and coordinate all human functions in response to changes in the external environment, as well as the internal environment of the tissues. The nervous system innervates every organ of the body and utilizes the patterned neural network activity to control organ processes and direct immune and endocrine responses to coordinate and maintain proper physiologic balance and functioning, a term called homeostasis (Rice and Albrecht, 2008, The Senses: A Comprehensive Reference, Vol 6, Somatosensation, Gardner E and Kaas J H. San Diego: Academic Press. P. 1-32).

A majority of neurons are concentrated in the various nuclei of the CNS. The CNS functions to interpret the totality of sensory stimulation (input) and coordinate and deliver the proper motor response (output) for every situation. The PNS is composed of various nerves that connect all the tissues of the body to the CNS. The PNS acts to relay sensory stimulation (input to CNS) and motor response (output from CNS) from/to each organ and system to maintain overall homeostasis.

About half of the PNS axons arise from primary sensory neurons (loSN) with cell bodies located in ganglia near the spinal cord and brainstem. The loSN have a unique axonal structure with a peripheral axon portion that innervates to a peripheral terminal site, as well as a central axon portion that connects to the CNS spinal cord or brainstem, thereby connecting the peripheral tissues to the CNS circuits that process sensory input. A major part of the nervous system sensory input and processing involves conscious awareness of sensation and a directed motor response, whereas a great portion of sensory input is acted upon by the nervous system, but without a conscious perception of the sensory input or drive of the response, only the perceptions of the result (e.g., wet mouth from salivating, cool skin from sweating). The other half of PNS axons in peripheral nerves arise from motor (output) neurons that project large peripheral axons to skeletal muscle and produce volitional movement (behavior) responses, as well as smaller axons from the autonomic nervous system (ANS) that mediate essential functions like respiration, cardiovascular performance, GI motility, peripheral blood flow regulation, and glandular responses, like salivating and sweating. The coordinated neural network functions of the CNS and PNS maintain homeostasis and promote survival under fluctuating situations and unknown stressors through a constant process of sensory input, neural signal pattern recognition, and directed motor output.

Somatosensory and Pain Pathways. Numerous parallel somatosensory pathways, including those that sense pain, transduce sensory stimuli and activate specific CNS circuits to provide sensory perceptions of the body. Somatosensory pathways generally consist of three sequentially connected neurons whose axons span from every tissue in the body to the cerebral cortex of the CNS (Rice and Albrecht, 2008, ibid). Sensory input begins through stimulus energy transduction to the peripheral terminals of loSN axons and the generation of an AP in the PNS nerves. The AP is transferred via synaptic transmission to a second order neuron located in the CNS spinal cord with axons that conduct the AP to the thalamus. The AP is then transferred via synaptic transmission to a 3rd neuron located in the thalamus with axons that terminate in specific regions of the cerebral cortex. The process of somatosensation, including pain, occurs with 5 distinct processes: 1) Stimulus Transduction, 2) AP Conduction, 3) Synaptic Transmission, 4) Signal Modulation, and 5) Cortical Pattern Recognition (Perception).

Non-pain somatosensory pathways are activated by various physical properties of objects in contact with the skin such as shape, firmness, texture, and temperature. The resultant patterns of cerebral cortex neural activity generate a conscious perception of touch that directly relates to the physical properties of the object. Other somatosensory pathways detect the position of the digits, limbs and body parts, with the patterns of cortical activity driving a conscious perception that relates to the position in space. Additional non-pain sensory pathways sense the biochemical functioning status of body tissues, including internal organs, muscles, vasculature, and skin, for which the patterns of CNS activity monitor and maintain proper balance, i.e., normal homeostasis.

Specific PNS loSN that activate CNS pain pathways are biochemically and structurally specialized to be activated by specific noxious stimuli from existing or potential physical damage to body tissues. These specialized loSN are called nociceptors (i.e., pain receptors) and AP generation on these axons is termed nociception to delineate the electrochemical signal from the conscious perception of pain, which only occurs after cerebral cortex patterned activation. The perception of pain from nociceptor activation of CNS pain pathways and specific cerebral cortex activation can be experienced across all of the somatosensory modalities (vision, hearing, taste, smell, touch).

Pain is a Complex Homeostatic Perception. The neural circuits that develop and function in the CNS and PNS detect, monitor, and respond to a vast array of subconscious sensory input regarding body systems to maintain homeostasis (Rice and Albrecht, 2008, ibid). Protective responses from noxious stimuli remain universal across the animal kingdom, and the complex psychologic perception of pain in humans serves an essential survival mechanism. In humans, the nervous system also creates a conscious perception (self-awareness) in relation to the constantly changing environment (external and internal). In any situation where sensory input and/or homeostasis is skewed beyond a normal range of functioning, particularly with impending or real tissue damage, an unpleasant, aversive, and motivative conscious sensory percept is created, called pain. Human pain represents a culmination of noxious sensory input from activation of PNS nociceptors (monitoring external and internal environments), expectation of the events from past experience, coupled with novel information, to create a comprehensive perception of pain/discomfort/fear/anxiety—to direct avoidance behaviors, promote tissue healing, and learning of potentially harmful situations.

The conscious perception of pain is also known to be parsed in the brain into an emotional component and a physical component, both with a wide range of subjective human perception and experience. Although motivative (behavioral) activities are often similar among individuals responding to a similar noxious stimuli (i.e., hand withdraw from fire), no two people can experience the exact same pain because it is an individual perception, modulated and influenced by innumerable factors, including individual psychology and group sociology. As such, pain perception is considered to be a biopsychosocial percept.

Pain Mechanisms. Following any type of potential or real tissue injury or infection/inflammation, a painful percept is generated that lasts until the damage is repaired or the threat is avoided. This normal, transient, acute pain warns about ongoing damage or pending threats to the body. The nociceptive molecular mechanisms associated with the generation of acute pain are well-established and several medications exist that can reduce acute pain during the tissue recovery and inflammation phases.

Acute nociceptive pain perceptions are adaptive cognitive survival mechanisms that are temporary (acute; <3 months) in providing an essential protective function for detecting incipient or overt damage in body tissues, motivating behavioral action to avoid or limit the damage, and then ceasing when damage is avoided and/or tissues are repaired. Acute pain may be associated with injury, surgery, inflammation, or any threat of tissue injury. Acute pain generally occurs with a sudden onset and terminates in a manner associated with threat avoidance and/or tissue healing. Normal acute pain activity is initiated and sustained by nociceptive molecular mechanisms being stimulated in the loSN of the PNS portion of the pain pathway, which are subsequently perceived as painful when conveyed to central nociceptive circuits and the cerebral cortex. Acute pain can be effectively managed with several classes of pharmaceuticals that either block activation of neurons that drive painful perceptions (i.e., nociceptors) or that activate endogenous pain reducing circuits (e.g., opiates). In particular, common existing medications for acute pain temporarily block specific molecular mechanisms associated with the membrane lipid cyclooxygenase (COX) signaling system. Inhibiting the production of COX intermediate signaling factors that activate and sensitize the peripheral nociceptors and central circuits associated with driving pain perception leads to a significant analgesic response. Opiates are highly effective for acute pain management, and act by stimulating an endogenous pain modulatory signaling system. Activation of the complex opiate system remains the most efficacious to mitigate acute pain perceptions, however the opiate system is also highly involved in other essential homeostatic functions, including respiration, and therefore pharmaceutical manipulation is often dangerous and limited by potentially fatal side-effects.

Disruption of homeostasis and/or overt tissue damage can occur following many varied situations, and recovery can be limited and/or incomplete. In particular, many people suffer from persistent and debilitating chronic pain conditions characterized by pain perceptions that continue after the tissue damage is healed and/or the threat avoided. In select cases of chronic pain, permanent damage to the nervous system is identified to be causative, and is therefore called neuropathic pain (NP) to distinguish it from an acute nociceptive pain process. NP conditions are most often chronic, and currently there are very few, mostly insufficient, treatment options for chronic NP conditions. As well, in many chronic pain conditions, the homeostatic skew responsible for the pain perceptions remains unknown. As such, chronic pain remains a pervasive and debilitating clinical problem for all patients afflicted with FM, PTSD, ME/CFS, IBS, post-viral and post-war syndromes, and other afflictions characterized by similar MUS (Albrecht and Rice, 2016, ibid).

Neuropathic pain (NP) is attributable to direct nerve pathologies, as with peripheral neuropathies and often soft tissue trauma. Any condition that causes abnormal nerve transmission, including metabolic disease, infection, ischemia, injury, entrapment, connective tissue disease, immunodeficiency, malignancy, drugs, and toxins can result in neuropathic changes and pain. In the absence of definitive evidence for direct neuron pathology, FM, PTSD, ME/CFS, IBS, post-viral and post-war syndromes, and other afflictions with MUS, have not been regarded as neuropathic. Rather, in many instances, these afflictions are presumed to have an unidentified CNS pathophysiological origin (e.g., central sensitization), and/or are commonly dismissed as psychosomatic malingering (Albrecht and Rice, 2016, ibid). Aspects of the present disclosure are based on the discovery of a direct PNS neuropathology in FM and PTSD, indicating that they are very likely peripheral NP disorders (Albrecht et., 2013, ibid).

Pain perceptions that continue beyond the avoidance of threat or the complete healing of injured tissue and resolution of inflammation is considered chronic pain (CP). CP is debilitating and maladaptive in that it no longer serves a survival or protective function. Some CP afflictions are attributed to irreparable and/or continuing tissue damage or disease (e.g., osteoarthritis, cancer), or have defined neuropathologies involving peripheral nerves (e.g., sciatica, carpal tunnel syndrome). Pathologies such as tumors or strokes directly impacting CNS neurons of pain pathways can also produce chronic perceptions of pain. Other CP afflictions develop following an initial tissue damaging event and subsequent healing, such as post-viral infections like COVID-19 and post-COVID-19 symptoms, often referred to as “long COVID.” Long COVID is often characterized by symptoms including fatigue, shortness of breath, brain fog, and sleep disorders. Additionally, several syndrome type CP afflictions (e.g., fibromyalgia, PTSD) have been attributed to pathologies within the CNS that create heightened nociceptive pathway stimulation, termed central sensitization. Central sensitization enhances normal peripheral input, thereby creating erroneous heightened misperceptions of homeostatic skew, leading to chronic conditions of widespread discomfort and pain. Also, many debilitating and intractable CP afflictions have no identified association with an acute trauma or known origin. These types of inexplicable CP conditions remain a common complaint in clinical practice, as exemplified by the high incidence and MUS among FM, PTSD, ME/CFS, IBS, post-viral and post-war syndrome patients. Many of the same pharmaceuticals that are efficacious for acute pain remain largely ineffective (e.g., COX inhibitors) or have only limited utility (e.g., opiates) for use in treating CP conditions.

Neurological basis of neuropharmaceuticals and pain therapeutics. The functional basis of neural circuits and patterned activity, including conscious pain perceptions, are driven by several unique properties of neurons that enable networked cellular communication (Rice and Albrecht, ibid; Albrecht and Rice, 2010, Curr Pain Headache Rep 3:179-188, PMID: 20425194). As noted prior for the somatosensory system, pain perceptions follow a sequential process of 1) neuronal stimulus transduction, 2) axonal conduction, 3) synaptic transmission, 4) signal modulation, and 5) cortical pattern recognition (pain perception). Manipulation of each of these processes via pharmaceuticals can have beneficial effects on blocking painful perceptions.

Neuronal conduction is the process where an electrochemical signal, termed an action potential (AP) is directionally propagated by the rapid movement of negative and positive ions through molecular pores (ion channels) embedded in the axonal membranes. AP are rapidly self-propagating neuronal membrane depolarizations that travel over the length of the axons. At the axon terminal, the AP is transferred to the next neuron across a small gap termed the synaptic cleft (e.g., synapse) via synaptic transmission. Specific signaling chemicals (neurotransmitters) carry the electrical impulses across the synapse and stimulate an AP in the post-synaptic neuron or effector cell (muscle or gland). Sensory stimuli transduction consists of multi-molecular mechanisms that convert sensory stimuli into AP for conductance within the loSN of any specific neuronal circuit. Once activated, an AP has no inherent meaning as a stand-alone signal, but the collective timing and activity patterns among the various neural circuits produce conscious perceptions and functional outcomes. Additionally, AP are under constant signal modulation by other active circuits at each synapse to shape the incoming signal for cortical recognition.

Pharmaceutical manipulation of sensory stimuli transduction, axon conductance, synaptic transmission, and/or signal modulation can lead to a diverse range of effects, including pain relief (analgesia). Consequently, the development of neuropharmaceuticals is based on the molecular properties associated with neural translation, conductance, transmission, and modulation. Therapeutic strategies for most neurological diseases, including chronic pain, are focused at manipulating some aspect of the molecular mechanisms associated with sensory transduction, AP conductance, synaptic transmission, and AP modulation.

Within a neuron, AP are generated and directionally conducted by the rapid movement of ions acting to establish equilibrium against a normal axon resting membrane potential of ˜-70 mV. Voltage-gated ion channels embedded in specific locations along the length of the axon membrane open in response to changes in membrane voltage and allow for the rapid influx of ions. The rapid depolarization of the axon membrane pushes the voltage changes down the axon and subsequently continues to open channels along the axon membrane, thereby propagating the AP along the axon length. Aberrant AP patterns driven by physical trauma and/or disease processes directly involving nociceptive neuron axons can generate chronic pain perceptions in the cerebral cortex. Specific potent anesthetics, for example lidocaine, directly block the voltage-gated ion channels in axons that propagate AP, and thus can suppress aberrant activity in neurons. Anesthetic therapeutics stop all axon conductance (sensory and motor) and therefore are quite useful for managing acute pain conditions during surgical procedures or for relief for chronic pain that is localized to a specific body location that does not require movements. However, these types of ion channel conduction blockers are not useful as analgesics when movement is still required, and therefore have very limited utility in treating the chronic pain associated with FM, PTSD, ME/CFS, IBS, and post-viral and post-war syndromes.

Many pharmaceutical strategies to treat a wide variety of neurological disorders, including chronic pain, are designed to target molecular mechanisms involved in neuronal synaptic transmission (Rice and Albrecht, 2008, ibid; Albrecht and Rice, 2010, ibid). Other common pharmaceutical treatments for pain conditions, and many OTC therapies, also interfere with sensory stimuli transduction to the 1° SN, thereby blocking the generation of an AP in a nociceptive neuron. Many topical pain relievers have transduction inhibition properties, for example capsaicin.

Neuronal Function. The molecular mechanisms of synaptic transmission pass an active AP from a presynaptic neuron across the synapse to initiate AP conduction in a post-synaptic neuron axon. In forming neuronal circuits, the tips of axons that connect neurons to each other physically end at their synaptic contacts, leaving a narrow ˜80 nanometer synaptic cleft. Synaptic transmission is effected through a multi-step multimolecular process in which the AP in the presynaptic neuron triggers the release of specific small molecules, called neurotransmitters (NT), from the synaptic tips of their axons. The released NT rapidly diffuse across the synaptic cleft and bind to specific molecular receptors in the postsynaptic neuron membrane. NT receptor binding stimulates the opening of ion channels in the post-synaptic neuron and the generation of a propagated AP. Synaptic transmission is repeated from neuron to neuron using a variety of different NT to complete the wide functional varieties of different nervous system circuits and pattern generation.

Neurons utilize genetically-programmed diversity in molecular expression of various neurotransmitters (NT) and receptors that imparts functional specificity to the synaptic transmission. Different types of neurons utilize specific NT to effect synaptic communication. Most neurons are genetically-programmed to synthesize a particular combination of NTs and are characterized by their specific NT expressions. Most NT have a stimulatory effect on the post-synaptic AP generation, called excitatory NT (e.g., glutamate), whereas several NT cause a decrease in the AP generation, called inhibitory NT (e.g., GABA). In addition to specific NT expressed by neurons for synaptic communication, a large variety of small molecule neuropeptides (e.g., Calcitonin Gene-related Peptide, CGRP) are also expressed and released to signal various end organs and structures (i.e., smooth muscle, glands). Currently, over 60 neurotransmitters and small molecules and their cognate receptor signaling systems have been identified, providing great diversity among neuronal populations.

Generation of AP and synaptic transmission across neural circuits are rapid transient events in which the strength of signaling is not due to AP magnitude, but rather AP frequency. As such, stronger stimulating events produce an increased number of AP conducted in the axons of the stimulated neurons. As each AP arrives at the synapse, it triggers a brief rapid release of neurotransmitter into the synapse. Consequently, a higher frequency of impulses triggers a matching frequency release of neurotransmitter to the subsequent neurons of the circuit.

Rate-limiting factors of synaptic transmission. Specific NT are constantly synthesized by neurons and available for synaptic transmission. A high sustained frequency of impulses can exceed and deplete the availability of NT within a neuron and therefore reduce the frequency of AP in the postsynaptic neurons of circuits. As well, following each AP, the released NT in the synaptic cleft binds to the molecular receptors in the postsynaptic neurons and initiates a response. Each AP-driven NT/receptor interaction has to be rapidly removed from the membrane and the synapse cleared, so the receptors can be available to respond to the next release of NT. Inhibition of any of the mechanisms associated with synaptic transmission function can modulate AP strength, and with regards to the somatosensory system and pain, alter perceptual qualities of a stimulus. There are two major mechanisms for reducing the rate-limiting restrictions of neuronal AP propagation. With some NT, for example acetylcholine (ACh), the postsynaptic neurons produce enzymes that rapidly degrade any unbound NT, thereby enabling rapid recovery and the continued ability to detect NT release from subsequent AP. For other NT, for example serotonin (5-HT) and norepinephrine (NE), the presynaptic neurons have specific transporter proteins that re-uptake the NT from the synaptic cleft into the presynaptic axon terminals.

Certain aspects of the present disclosure are based from several unique discoveries of PNS normal neurovascular innervation organization and neuropathology in select patients (Rice and Rasmussen, 2000, J Comp Neurol 417:467-490. PMID: 10701867; Bowsher et al., 2009, Pain 147:287-298. PMID: 19836135; Albrecht et al., 2013, ibid). The discovery and neurochemical characterization of a unique neuropathology in FM and PTSD provided a novel target and non-intuitive mechanistic rationale for potential treatments. By targeting these peripheral pathologic mechanisms using a unique combination of FDA-approved pharmaceuticals, greater efficacy and limited side-effects can be achieved. Based on the properties of the neuropathology, the methods and combinations described herein make novel use of at least two types of existing pharmaceuticals that each act at three different functional steps as part of the discovered FM and PTSD pathology: 1) CGRP release from presynaptic neurons, 2) CGRP availability in the synaptic cleft, and 3) CGRP activation of receptors on postsynaptic targets (see FIG. 2). The existing pharmaceuticals are Serotonin-Norepinephrine Reuptake Inhibitors (SNRI) and Calcitonin Gene-Related Peptide (CGRP) signaling inhibitors (see FIG. 2).

SNRI. In some embodiments, the therapeutic combinations described herein comprise pharmaceuticals referred to as SNRI. The SNRI duloxetine (manufactured under the name Cymbalta®) was developed and approved by the US FDA for major depressive disorders (MDD), but has seen off-label use in chronic pain. The SNRI milnacipran (manufactured under the name Sevella®) is approved by the European Union for MDD and in the US for the treatment of FM, but is often discontinued in FM due to increased adverse events. The development of SNRI was based from CNS circuits involving 5-HT and NE, for which abnormally low levels of synaptic signaling were implicated in MDD. SNRI were developed to prolong the activity of released 5-HT and/or NE within the synapse by inhibiting the molecular re-uptake mechanism. Although there were no initial indications of SNRI functionality for CP conditions, off-label empirical use demonstrated SNRI efficacy in certain CP conditions, particularly FM. Currently, the modest efficacy of SNRI for CP remains limited due to adverse side-effects, possibly due to disturbance of normally functioning 5-HT and/or NE signaling mechanisms.

CGRP. A select population of PNS loSN produce the neuropeptide CGRP and are called peptidergic neurons. CGRP is released from the synaptic contacts of peptidergic axons in the spinal cord and brainstem, in addition to the NT glutamate. In these specialized peptidergic loSN, CGRP can also be released from the peripheral terminals to effect responses in innervated tissues. Since the time of its discovery in the 1980s, CGRP has been known to be involved in numerous biologic processes, and specific peptidergic CGRP loSN subgroups serve different tissue needs, including inflammatory pain processes. As such, CGRP has been a target of pharmaceutical research for treating CP. Peptidergic CGRP loSN are essential to normal pain pathways, and in certain CP conditions, alterations of the CGRP system have been identified (Albrecht and Rice, 2010, ibid). CGRP effects are mediated via a specific multi-molecular receptor complex consisting of a calcitonin receptor-like receptor (CRLR) protein, a co-receptor protein designated receptor activity-modifying protein 1 (RAMP1), and the CGRP receptor component protein (RCP), as a distinct CRLR/RAMP1/RCP complex to initiate downstream signaling events. Some types of peptidergic CGRP loSN have peripheral terminations in skeletal muscle, and these axons also express specific Acid Sensing Ion Channels (ASIC). For example, ASIC3 is activated by lactic acid, a skeletal muscle activity-byproduct that can shift muscle tissue to anaerobic metabolism when the capillary supply of oxygen is insufficient to sustain prolonged exercise (Molliver et al., 2005, Mol. Pain 1:35:1-13. PMID: 16305749). As such, the activation of these ASIC3/CGRP loSN are implicated in the perception of muscle aches and fatigue, as a signal for the need to rest and recover to normal levels of oxidative metabolism.

In some subgroups of CGRP loSN, CGRP is released from the sensory neuron peripheral endings. Peripherally released CGRP is known to mediate a variety of functions among local cells that express the CRLR/RAMP1/RCP complex, including the process of neurogenic inflammation. For example, CGRP loSN subtypes that terminate in the epidermis may release CGRP to maintain epidermal homeostasis, as CGRP promotes the normal proliferation of epidermal keratinocytes and modulates epidermal Langerhans Cell function. Importantly, CGRP activation of the CRLR/RAMP1/RCP complex expressed by capillary endothelial cells and by arteriole and arteriovenous shunt (AVS) smooth muscle cells leads to significant relaxation, and CGRP signaling is considered to be one of the most potent vasodilatory mechanisms. Numerous CGRP loSN terminate among capillaries in the skin where the CGRP vasodilatory properties contribute to redness (flare) and edema (wheal) associated with inflammation and pain in injured skin.

CGRP inhibitors. A select subgroup of peptidergic CGRP loSN terminate extensively among capillaries in the dura mater. CGRP release from these terminals is thought to be a major source of elevated CGRP blood levels known to occur during migraine headache events (Albrecht et al., 2010, ibid; Rice et al. 2016, Cephalalgia 37:1250-1272, PMID: 27852962). Recently, several types of CGRP signaling inhibitors gained FDA-approval for the treatment of migraine headache, after decades of attempts to develop CGRP-related therapeutics for several CP disorders. The specific inhibitors target the CGRP ligand and/or the receptor complex with monoclonal antibodies or small molecules. However, given the numerous locations and diverse functional roles of CGRP signaling, several CGRP inhibitors have limiting adverse effects, particularly constipation, likely due to CGRP modulation of GI function.

SNRI pharmaceuticals were developed for treatment of MDD based from evidence of dysfunctional signaling among 5-HT and NE neurons in the CNS. The efficacy of SNRI for pain relief in FM was based on empirical off-label use. The partial effectiveness of SNRI in treating FM pain contributes to the prevailing hypothesis that FM is a CNS disorder (i.e., central sensitization), particularly since existing evidence of peripheral pathology was unknown prior to the novel discovery among the inventors described herein (Albrecht et al., 2013, ibid) (see FIG. 1). Additionally, there had been no mechanistic evidence for SNRI involvement in the PNS. However, the inventors have an extensive research history on normal skin innervation in many different species, including humans, which led them to recognize that there was a potential site for SNRI effects among the PNS neurovascular innervation to cutaneous arterioles and AVS (Rice and Albrecht, 2008, ibid; Bowsher et al., 2009, ibid). Specifically, among the vascular bed arterioles and AVS, a PNS convergence of peptidergic CGRP loSN innervation and sympathetic NE innervation occurs, most likely to modulate vascular tone and blood flow dynamics. Follow-up research in FM patients led to the discovery of a significant neurovascular pathology, which was subsequently also observed in a small cohort of PTSD patients (see FIG. 1). Characterization of the peripheral neurovascular pathology demonstrated an increased proportion of CGRP innervation, and confirmed that peripheral activity of an SNRI compound could modulate the pathology and possibly derive clinical benefit (see FIGS. 1 and 2).

Sympathetic NE innervation and vasoconstriction. Neurovascular innervation plays an essential role in regulating blood flow to balance fluctuations in activity-related demands for nutrition (such as increases in skeletal muscle during exercise) and thermal conditions (such as increased metabolic heat from exercise and changes in external temperatures) (Albrecht et al. 2013, ibid). The ability to properly regulate blood flow and apportioning is essential to homeostatic mechanisms during which a constant oxygen and nutrient supply is required, as well as a maintenance of core temperature to provide proper functioning of organs. The non-conscious ANS is a major part of the PNS that innervates and regulates visceral motor functions, including neurovascular regulation. In maintaining normal homeostasis, blood flow through the capillary beds of all body tissues is where demands are met for oxygen, cellular nutrition, and for the conservation or dissipation of heat at the skin surface in order to maintain a constant core temperature. Blood flow to the capillaries, as needed, is overwhelmingly controlled at the level of arterioles, AVS, and precapillary arterioles whose vessel walls contain smooth muscle that can constrict or dilate to respectively reduce or increase blood flow through the capillary beds. Vasoconstriction is mediated primarily by a subset of ANS sympathetic neurons that release NE at their vascular axon terminals, which in turn binds to NE receptors on the smooth muscle to effect contraction (see FIGS. 1 and 2). In facial skin and brain vasculature, other ANS neurons (referred to as parasympathetic) terminate on arterioles to effect dilation of the smooth muscle through the release and receptor binding of the neurotransmitter acetylcholine. However, parasympathetic innervation is lacking among arterioles, AVS, and precapillaries throughout the body. As such, body-wide neurovascular organization and modeling has been largely based on the assumption that activation of NE sympathetic innervation mediates vasoconstriction, and vasodilatation has been attributed to the relaxation of smooth muscles via nitric oxide (NO) passively released from endothelial cells in response to smooth muscle contraction.

CGRP loSN and vasodilation. Many types of loSN use CGRP as a signaling mediator, which is released from their synaptic contacts in the spinal cord and brainstem, and implicated as part of pain pathways. CGRP was initially demonstrated to be a potent vasodilator in skin and is implicated in inflammatory pain-related dilation of capillaries after injuries. Additionally, CGRP loSN had been shown to innervate arterioles, however the innervation was largely thought to sense vascular tone, and not to have a direct effector function. For example, the discovery that ASIC3/CGRP loSN innervate among blood vessels in skeletal muscle was attributed to those axons being functional metabolic receptors, implicated in perceptions of normal muscle aches and fatigue due to lactic acid build-up from activity (Molliver et al., 2005, ibid). As well, CGRP loSN to the dura mater had been demonstrated and the vasodilation theory of migraine supported a direct role of CGRP in the pathophysiology. Recently, several anti-CGRP therapies have now been FDA-approved for use in migraine treatment. However, the potential role of CGRP-release from peptidergic loSN on body-wide arterioles, AVS, and precapillary arterioles remains largely ignored as a mechanism in normal vasoregulation, and has not been considered as a potential source of the MUS related to FM and PTSD, including CP, fatigue, non-restorative sleep, and cognitive dysfunction.

NE sympathetic inhibition and CGRP loSN dilation in normal vasoregulation. Comprehensive characterization of the discovered neurovascular pathology demonstrated excessive CGRP loSN terminals on the AVS in the palmar skin of FM patients (Albrecht et al., 2013, ibid) (see FIG. 1). CGRP released from these excessive endings would likely contribute to blood flow dysregulation and an increase in dilation tone. Importantly, the peptidergic CGRP loSN terminations on cutaneous arterioles and AVS expressed a specific type of NE receptor subtype, referred to as alpha receptors (Albrecht et al., 2013, ibid; Rice et al., 2016, ibid). Several types of alpha receptors activated by NE binding result in effector cell stimulation, for example alpha-1 (aA1) receptors on arteriole and AVS smooth muscle cells mediate NE-stimulated vasoconstriction. Comprehensive characterization of the arteriole and AVS neurovascular pathology innervation from FM and PTSD patients demonstrated that the peptidergic CGRP innervation expressed the alpha2C (a2C) subtype of NE receptors (Albrecht et al., 2013, ibid). NE binding to a2C receptors results in effector cell inhibition and a2C activation been shown to inhibit CGRP release from the peripheral axon terminals. The combined observations indicate that normal vasoregulation is likely controlled by the balance of vasoconstriction effects of sympathetic NE and vasodilation effects of sensory CGRP from the loSN terminals (see FIG. 1). NE can also inhibit the release of vasodilatory CGRP via alpha2C receptors, thereby increasing the vasoconstrictive effects of released NE. Taken altogether, this data demonstrates a potential mechanism for an SNRI impact among the aberrant peripheral innervation to AVS, which would reduce the level of the vasodilatory molecule CGRP, thereby restoring normal vascular homeostasis (see FIGS. 1 and 2). Once vascular dynamics are corrected, it is hypothesized that a resolution of pain mechanisms, fatigue, non-restorative sleep, and cognitive dysfunction will be observed.

A novel peripheral mechanism for the MUS of FM patients. The discovery of excessive CGRP loSN innervation in the palmar skin AVS of FM patients by the inventors provides a possible rationale for the extreme tenderness in FM patient hands and particularly widespread pain, fatigue, non-restorative sleep, and cognitive dysfunction (Albrecht et al., 2013, ibid). AVS are mostly located in the hands and feet in humans, and are essential structures for total body blood apportioning and flow dynamics. Under the pathological conditions of excessive CGRP loSN innervation in FM patient AVS, the NE sympathetic innervation may be insufficient to prevent or override an excessive release of CGRP, resulting in increased AVS dilation, limited function for proper blood apportioning, and a potential to develop MUS associated with FM, PTSD, ME/CSF, IBS, post-viral and post-war syndromes. Because, a NE-mediated A2C activation would inhibit CGRP release, SNRI may slow NE reuptake, enabling prolonged availability of NE to reduce CGRP release from the excessive CGRP loSN innervation (see FIGS. 1 and 2).

Several previously unexplained phenomena provide a unified insight into the debilitating MUS of widespread pain, fatigue, and also non-restorative sleep and cognitive dysfunctions as an indirect consequence of systemic vasoregulatory dysfunction and misappropriation of blood flow (Albrecht et al., 2013, ibid). In particular, the hands and feet have a high surface to volume ratio, and are the location with a high density of AVS structures. These glabrous skin locations function under normal conditions as important thermoregulatory locations, in which blood flow is increased to the capillary beds during the need to dissipate heat, or restricted from the capillary beds to conserve heat and maintain core temperature. The AVS vascular structures and large capillary beds of the glabrous skin in the hands and feet allow for constant subtle variations in blood volume, apportioning, and flow dynamics. For example, blood flow is largely apportioned to skeletal muscle during increased aerobic physical activity. After continued activity, the increased metabolic heat needs to be dissipated, and blood flow is apportioned back toward the skin capillary beds of hands and feet. Under normal physiology, these processes act to maintain appropriate vascular homeostasis (flow, volume apportioning) on a moment-to-moment basis. Concomitantly, skeletal muscle transitions to anerobic metabolism and produces a consequent increase in lactic acid. This triggers activation of the skeletal muscle ASIC/CGRP loSN that are implicated in the normal perception of muscle aching and fatigue that normally dissipates with rest and restoration of aerobic metabolism. These are precisely the perceptions that are exacerbated and persistently debilitating in FM, PTSD, and particularly ME/CFS patients. These phenomena highlight the AVS as essential structural mediators of the normal metabolic- and thermoregulatory-driven vascular dynamics required to maintain homeostasis under constantly varying environmental (external and internal) conditions. Structurally, AVS create a direct vascular connection between arterioles and venules, as a bypass to pushing arteriole (oxygenated) blood flow through a capillary bed and back into venous (nonoxygenated) blood. The activity of sympathetic NE-mediated AVS constriction directs blood flow to the skin capillaries, for example to dissipate heat. Activity of sensory CGRP-mediated AVS dilation diverts blood flow away from skin capillaries. These systems function in dynamic combination to maintain vascular tone and proper blood apportioning to match metabolic and thermal demands. Characterization of the discovered neuropathology in FM and PTSD patients demonstrated an excessive sensory CGRP vasodilatory innervation that far exceeded the sympathetic NE vasoconstictive innervation, and therefore the AVS are likely in a pathological constantly partially dilated state (i.e., skewed homeostatic vascular tone), and also are unable to respond properly to metabolic or thermal challenges, or required alterations in blood flow dynamics during sleep and for normal cognitive functioning. The discovered AVS neuropathology would compromise needed blood flow to skeletal muscle resulting in constant anaerobic conditions and lactic acid build-up, triggering a perception of chronic wide spread muscle aching and fatigue. As well, this AVS pathological mechanism likely also accounts for exacerbated weather-related symptoms in FM patients, particularly temperature extremes, hot or cold. Furthermore, the AVS neuropathology likely also leads to a mismanagement of blood flow during sleep resulting in non-restorative sleep and fatigue. Similarly, improper blood flow dynamics to the brain may impact normal cognitive function in FM patients. As such, a major benefit of SNRI treatment could be due to peripheral mechanisms associated with vascular AVS function and blood flow dynamics. Action of the SNRI to increase the amount of NE available to bind at the AVS would enhance the direct constrictive function, and also provide an indirect blocking function of the release of CGRP from the sensory innervation. These SNRI activities likely act to restore a more balanced function to the skewed AVS and promote vascular homeostasis.

Certain aspects of the present disclosure are based from a comprehensive neurovascular innervation characterization and tissue structure analysis of glabrous skin. The ChemoMorphometric Analysis technology utilized allowed for the discovery of a unique and functionally important neuropathology. In doing so, the inventors utilized their unobvious expertise and apriori knowledge to hypothesize where to look in the PNS for a site of SNRI impact (i.e., vascular AVS of glabrous skin), and characterize the unique molecular properties associated with the discovered neurovascular pathology. Furthermore, detailed understanding of vascular function allowed for the recognition that the AVS innervation pathology provides a plausible global mechanism for the MUS of widespread pain, fatigue, non-restorative sleep, and cognitive dysfunction observed in FM, PTSD, ME/CFS, IBS, and others. A detailed knowledge of pharmacology allowed for the recognition of how an appropriate combination of existing therapeutic compounds (as provided herein, in some embodiments) may improve the MUS associated with FM and other disorders that have an underlying similar neurovascular pathology, while minimizing adverse events commonly associated with the use of these compounds. A subsequent discovery of a similar neurovascular pathology in PTSD patients, with identical MUS of FM patients, provides additional rationale for testing the combination strategy on FM and PTSD patients, as well as and other patients with similar MUS, for example ME/CFS, IBS, or post-viral and post-war syndromes.

In summary, aspects of the present disclosure are based on the following novel discoveries, interpretation, and theory (see FIGS. 1 and 2): 1) AVS in humans play an important role in regulating blood flow to adjust for daily dynamics in metabolic and thermal demands; 2) homeostatic blood flow regulation and volume apportioning by AVS is normally achieved by maintaining appropriate vascular tone via proportions of direct smooth muscle vasodilation mediated by CGRP release from loSN terminals and vasoconstriction mediated by NE release from sympathetic terminals; 3) sympathetic NE innervation also modulates vasodilation via binding to inhibitory a2C receptors on loSN terminals to inhibit the release of CGRP; 4) FM patients have a pathologically extreme excess of CGRP loSN terminals on AVS for which the NE innervation would be insufficient to inhibit excessive release of vasodilatory CGRP, resulting in an AVS with increased vasodilatory tone; 5) the AVS vasodilated patency would compromise normal vasoregulation resulting in insufficient blood flow to skeletal muscles, increased anaerobic production of lactic acid, hyperactivity of skeletal muscle afferents, and chronic wide spread pain and fatigue; 6) AVS dysregulation would compromise vascular dynamics associated with changes in ambient temperatures contributing to fatigue and non-restorative sleep; 7) AVS dysregulation during sleep and waking cycles would compromise oxygenation levels of the brain and contribute to non-restorative sleep and cognitive dysfunctions.

Provided herein are combinations of non-opiate pharmaceuticals that have different mechanistic approaches to reduce CGRP release from loSN innervation terminals among AVS, which normally have a convergence of sympathetic NE and sensory CGRP innervation in an approximate 1:1 ratio (see FIG. 2). The rationale for the specific compound combinations is based on the discovery of excessive sensory CGRP innervation to AVS in FM and PTSD patients. The discovered neurovascular pathology showed a significant excess of sensory CGRP vasodilatory innervation compared with sympathetic NE innervation. The sympathetic NE innervation may normally play a role in regulating the activity of the sensory CGRP innervation, and together these systems maintain a homeostatic balance of vasodilation and constriction (Albrecht and Rice, 2016, ibid). Proper AVS blood flow regulation and volume apportioning are essential for microvascular blood flow dynamics to maintain oxygen, metabolic, and thermoregulatory demands of tissues. Each of the individual compounds used in embodiments of the combinations described herein has been previously FDA-approved for systemic delivery. Specifically, SNRI may partially reduce pain among many FM patients, and CGRP inhibitors may reduce the incidence or severity of migraine headaches. Each of the drug classes described herein is mechanistically different and has significant side-effects when used alone at their approved systemic dosages. Certain aspects of the present disclosure make use of the discovered expression of a2C among the sensory CGRP innervation to AVS. The disclosure provides for combinations of pharmaceutical compounds that will have additive or synergistic effects, thereby treating the underlying vascular regulation pathology leading to a reduction in the MUS of chronic widespread pain, fatigue, non-restorative sleep, and cognitive dysfunction, but at relatively lower individual doses to limit adverse events and increase the therapeutic window of these compounds.

Some embodiments of the compositions and methods described herein are based on a strategy to address three aspects of the excess CGRP pathology: 1) reduce CGRP release, 2) reduce availability of CGRP that has been released, and 3) reduce the ability of available CGRP to activate the CRLR/RAMP1/RCP receptor complex. In some embodiments, each of these aspects is addressed in incremental steps to be sure not to overwhelm the CGRP signaling system at off-target locations.

### II. Formulations

In one aspect, provided herein are pharmaceutical compositions comprising at least two components (“active ingredient combination”) selected from the group consisting of: 1) a serotonin-norepinephrine reuptake inhibitor (SNRI); 2) a calcitonin gene-related peptide (CGRP) receptor antagonist; 3) an anti-CGRP monoclonal antibody; and 4) a specific alpha-2C agonist (see FIG. 2). As used herein, the term “pharmaceutical composition” refers to a mixture of components suited for administration to a subject. In some embodiments, a single formulation comprises all of the active ingredients. In some embodiments, the active ingredients are present in separate compound formulations intended for co-administration.

### 1. SNRI

In some embodiments, the pharmaceutical compositions comprise a serotonin-norepinephrine reuptake inhibitor (SNRI), a class of medication often used for depression. In some embodiments, SNRI compounds block the reabsorption (reuptake) of the neurotransmitters serotonin (5-HT) and norepinephrine (NE). In some embodiments, this may lead to inhibition of the release of CGRP from sensory fibers and a reduction of AVS dilation. SNRI compounds useful in the pharmaceutical compositions disclosed herein include, but are not limited to, duloxetine (Cymbalta, Irenka), milnacipran (Savella), venlafaxine (Effexor XR), levomilnacipran (Fetzima), desvenlafaxine (Pristiq, Khedezla), atomoxetine (Strattera), amitriptyline, nortriptyline, desipramine, or combinations thereof.

The SNRI compounds duloxetine and milnacipran have been approved by the FDA for the treatment of fibromyalgia. Administration of SNRI can lead to undesirable side-effects that limit efficacious utility. As used herein, the term “side-effect” refers to a secondary, typically undesirable adverse event directly caused by, or associated with, use of any targeted pharmaceutical compound or medical treatment. SNRI side-effects often include nausea, changes in appetite, muscle weakness, tremors, agitation, heart palpitations, increased blood pressure, increased heart rate, headache, difficulty urinating, dizziness, insomnia, sleepiness, dry mouth, excessive sweating, constipation, fluid retention, or changes in sexual function (e.g., reduced sexual desire, difficulty reaching orgasm, inability to maintain arousal).

### 2. CGRP Receptor Antagonist

In some embodiments, the pharmaceutical compositions disclosed herein comprise a small molecule calcitonin gene-related peptide (CGRP) receptor antagonist, compounds which, in some embodiments, block association of CGRP with the CGRP receptor complex (CRLR/RAMP1/RCP). In some embodiments, this may lead to reduction in vasodilation of the AVS. Small molecule CGRP receptor antagonists useful in the pharmaceutical compositions disclosed herein may include, but are not limited to, rimegepant (Nurtec ODT), ubrogepant (Ubrelvy), atogepant, BI 44370 TA, MK-3207, olcegepant, SB-268262, or telcagepant. In some embodiments, the CGRP receptor antagonist is a specific monoclonal antibody, such as erenumab (Aimovig).

The CGRP receptor antagonists ubrogepant and rimegepant have been approved by the FDA for treatment of migraines. Administration of CGRP receptor antagonists can lead to undesirable side-effects, including but not limited to allergic reactions (e.g., trouble breathing or rash), nausea, somnolence, dry mouth, fever, hives, reddening of the skin, swelling of the eyes, face or inside of the nose, or constipation.

### 3. Anti-CGRP Monoclonal Antibody

In some embodiments, the pharmaceutical compositions disclosed herein comprise an anti-CGRP monoclonal antibody. In some embodiments, anti-CGRP monoclonal antibodies bind to CGRP and prevent its association with the CGRP receptor complex (CRLR/RAMP1/RCP). In some embodiments, this interaction may lead to a reduction in vasodilation of the AVS. The anti-CGRP antibodies useful in the pharmaceutical compositions disclosed herein may include, but are not limited to, eptinezumab (Vyepti), fremanezumab (Ajovy), and galcanezumab (Emgality), all of which have been approved by the FDA for treatment and/or prevention of migraine headache.

Administration of anti-CGRP monoclonal antibodies can lead to undesirable side effects, which may include injection site reactions (e.g., redness, itchiness, pain, or tenderness), allergic reaction (e.g., rash, swelling of face, lips, tongue, or throat, trouble breathing, hives, redness), fatigue, stuffy nose, scratchy throat, fast heartbeat, fever, irritation, tightness in the chest, trouble swallowing, or joint pain.

### 4. Alpha-2C Agonist

In some embodiments, the pharmaceutical compositions disclosed herein comprise an alpha-2C agonist. In some embodiments, an alpha-2C agonist binds to the alpha-2C adrenergic receptor and inhibits CGRP release. In some embodiments, this may lead to a reduction in vasodilation of the AVS. The alpha-2C agonists useful in the pharmaceutical compositions disclosed herein may include, but are not limited to, clonidine (Catapres), (R)-3-nitrobiphenyline, tizanidine (Zanaflex), or dexmedetomidine (Precedex). In some embodiments, the alpha-2C agonist is specific for the alpha-2C receptor. In some embodiments, the alpha-2C agonist interacts with multiple alpha-2 adrenergic receptors.

Clonidine is FDA approved for hypertension, attention deficit hyperactivity disorder, Tourette syndrome tics, and as an adjunct therapy for cancer-related pain. Administration of alpha-2C agonists can lead to undesirable side effects, which may include abdominal pain, headache, hypotension, fatigue, nausea, emotional instability, constipation, xerostomia, diarrhea, sexual dysfunction, dizziness, sedation, depression, hypersensitivity, atrioventricular block, bradycardia, syncope, fever, or congestive heart failure.

### 5. Active Ingredient Combinations

The pharmaceutical compositions disclosed herein comprise a combination of at least two compounds selected from the categories consisting of: 1) a serotonin-norepinephrine reuptake inhibitor (SNRI); 2) a calcitonin gene-related peptide (CGRP) receptor antagonist; 3) an anti-CGRP monoclonal antibody; and 4) an alpha-2C agonist. Any combination of compounds from two, three, or four of the disclosed categories is contemplated. In some embodiments, multiple compounds are included from one or more of the at least two categories. For example, a pharmaceutical composition of the present disclosure may include two SNRI compounds and a CGRP receptor antagonist compound. As another example, a pharmaceutical composition of the present disclosure may include two SNRI compounds and two CGRP receptor antagonist compounds.

In some embodiments, the pharmaceutical composition comprises an SNRI (e.g., duloxetine, milnacipran, venlafaxine, levomilnacipran, desvenlafaxine, atomoxetine, or combinations thereof). In some embodiments, the pharmaceutical composition comprises a CGRP receptor antagonist (e.g., rimegepant, ubrogepant, atogepant, BI 44370 TA, MK-3207, olcegepant, SB-268262, telcagepant, erenumab, or combinations thereof). In some embodiments, the pharmaceutical composition comprises an anti-CGRP monoclonal antibody (e.g., eptinezumab, fremanezumab, galcanezumab, or combinations thereof). In some embodiments, the pharmaceutical composition comprises an alpha-2C agonist (e.g., clonidine, (R)-3-nitrobiphenyline, tizanidine, dexmedetomidine, or combinations thereof).

In some embodiments, the pharmaceutical composition comprises an SNRI and a CGRP receptor antagonist. In some embodiments, the pharmaceutical composition comprises an SNRI and an anti-CGRP monoclonal antibody. In some embodiments, the pharmaceutical composition comprises an SNRI and an alpha-2C agonist. In some embodiments, the pharmaceutical composition comprises an SNRI, a CGRP receptor antagonist, and an anti-CGRP monoclonal antibody. In some embodiments, the pharmaceutical composition comprises an SNRI, a CGRP receptor antagonist, and an alpha-2C agonist. In some embodiments, the pharmaceutical composition comprises an SNRI, an anti-CGRP monoclonal antibody, and an alpha-2C agonist. In some embodiments, the pharmaceutical composition comprises an SNRI, a CGRP receptor antagonist, an anti-CGRP monoclonal antibody, and an alpha-2C agonist.

In some embodiments, the pharmaceutical composition comprises a CGRP receptor antagonist and an anti-CGRP monoclonal antibody. In some embodiments, the pharmaceutical composition comprises a CGRP receptor antagonist and an alpha-2C agonist. In some embodiments, the pharmaceutical composition comprises a CGRP receptor antagonist, an anti-CGRP monoclonal antibody, and an alpha-2C agonist. In some embodiments, the pharmaceutical composition comprises an anti-CGRP monoclonal antibody and an alpha-2C agonist.

### 6. Pharmaceutically Acceptable Carriers

The formulations of the present disclosure may comprise, in addition to the active ingredient combination, a pharmaceutically acceptable carrier (excipient). A pharmaceutically acceptable carrier is a material that is not biologically or otherwise undesirable, i.e., the material is administered to a subject without causing undesirable biological effects or interacting in a deleterious manner with the other components of the pharmaceutical composition in which it is contained. The carrier is selected to minimize any degradation of the active ingredients and to minimize any adverse side effects in the subject. The pharmaceutical compositions may further comprise a diluent, solubilizer, emulsifier, preservative, and/or adjuvant to be used with the methods disclosed herein. Such pharmaceutical compositions can be used in a subject that would benefit from administration of a combination of any two of the active ingredients (e.g., a subject displaying symptoms of a chronic pain disorder, as detailed below).

Suitable carriers and their formulations are described in Remington: The Science and Practice of Pharmacy, 21st Edition, Philip P. Gerbino, ed., Lippincott Williams & Wilkins (2006). In certain embodiments, acceptable formulation materials preferably are nontoxic to recipients at the dosages and concentrations employed. In certain embodiments, the formulation material(s) are for subcutaneous and/or intravenous administration. In certain embodiments, the formulation comprises an appropriate amount of a pharmaceutically-acceptable salt to render the formulation isotonic. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. In certain embodiments, the optimal pharmaceutical composition is determined by one skilled in the art depending upon, for example, the intended route of administration, delivery format and desired dosage. See, for example, Remington: The Science and Practice of Pharmacy, 22nd Edition, Lloyd V. Allen, Jr., ed., The Pharmaceutical Press (2014). In certain embodiments, such compositions may influence the physical state, stability, rate of in vivo release and/or rate of in vivo clearance of the active ingredients.

In certain embodiments, the primary vehicle or carrier in a pharmaceutical composition can be either aqueous or non-aqueous in nature. For example, in certain embodiments, a suitable vehicle or carrier can be sterile water for injection, physiological saline solution, buffered solutions like Ringer's solution, dextrose solution, or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise a pH controlling buffer such phosphate-buffered saline or acetate-buffered saline. In certain embodiments, a composition comprising the active ingredient combinations disclosed herein can be prepared for storage by mixing the selected composition having the desired degree of purity with optional formulation agents (see Remington: The Science and Practice of Pharmacy, 22nd Edition, Lloyd V. Allen, Jr., ed., The Pharmaceutical Press (2014)) in the form of a lyophilized cake or an aqueous solution. Further, in certain embodiments, a composition comprising the active ingredient combinations disclosed herein can be formulated as a lyophilizate using appropriate excipients. In some instances, appropriate excipients may include a cryo-preservative, a bulking agent, a surfactant, or a combination of any thereof. Exemplary excipients include one or more of a polyol, a disaccharide, or a polysaccharide, such as, for example, mannitol, sorbitol, sucrose, trehalose, and dextran 40. In some instances, the cryo-preservative may be sucrose or trehalose. In some instances, the bulking agent may be glycine or mannitol. In one example, the surfactant may be a polysorbate such as, for example, polysorbate-20 or polysorbate-80.

In certain embodiments, the pharmaceutical composition can be selected for parenteral delivery (e.g., through injection by intravenous, intraperitoneal, intracerebral (intra-parenchymal), intracerebral, intraventricular, intramuscular, subcutaneous, intra-ocular, intraarterial, intraportal, or intralesional routes). Preparations for parenteral administration can be in the form of a pyrogen-free, parenterally acceptable aqueous solution (i.e., water, alcoholic/aqueous solutions, emulsions or suspensions, including saline and buffered media) comprising the active ingredients in a pharmaceutically acceptable vehicle. Preparations for parenteral administration can also include non-aqueous solutions, suspensions, and emulsions. Examples of non-aqueous solvents are propylene glycol, polyethylene glycol, vegetable oils such as olive oil, and injectable organic esters such as ethyl oleate. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product that can then be delivered via a depot injection. In certain embodiments, hyaluronic acid can also be used, and can have the effect of promoting sustained duration in the circulation.

In certain embodiments, the compositions can be selected for inhalation or for delivery through the digestive tract, such as orally (e.g., sublingually). Compositions for oral administration include powders or granules, suspension or solutions in water or non-aqueous media, capsules, sachets, or tables. Thickeners, flavorings, diluents, emulsifiers, dispersing aids or binders are optionally desirable.

In certain embodiments, the compositions can be selected for topical delivery. Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

In certain embodiments, the formulation components are present in concentrations that are acceptable to the site of administration. In certain embodiments, buffers are used to maintain the composition at physiological pH or at a slightly lower pH, typically within a pH range of from about 5 to about 8. For example, the pH may be 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 6.9, 7.0, 7.1, 7.2, 7.3, 7.4, 7.5, 7.6, 7.7, 7.8, 7.9, 8.0, 8.1, 8.2, 8.3, 8.4, or 8.5. In some instances, the pH of the pharmaceutical composition may be in the range of 6.6-8.5 such as, for example, 7.0-8.5, 6.6-7.2, 6.8-7.2, 6.8-7.4, 7.2-7.8, 7.0-7.5, 7.5-8.0, 7.2-8.2, 7.6-8.5, or 7.8-8.3. In some instances, the pH of the pharmaceutical composition may be in the range of 5.5-7.5 such as, for example, 5.5-5.8, 5.5-6.0, 5.7-6.2, 5.8-6.5, 6.0-6.5, 6.2-6.8, 6.5-7.0, 6.8-7.2, or 6.8-7.5.

In certain embodiments, a pharmaceutical composition can comprise a therapeutically effective amount of two or more active ingredients in a mixture with non-toxic excipients suitable for the manufacture of tablets. In certain embodiments, by dissolving the tablets in sterile water or other appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions can be selected by one skilled in the art, including formulations involving the active ingredient combinations disclosed herein in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, International Application Publication No. WO/1993/015722, which describes the controlled release of porous polymeric microparticles for the delivery of pharmaceutical compositions. In certain embodiments, sustained-release preparations can include semipermeable polymer matrices in the form of shaped articles, e.g., films, or microcapsules. Sustained release matrices can include polyesters, hydrogels, polylactides (see, e.g., U.S. Pat. Nos. 3,773,919; 5,594,091; 8,383,153; 4,767,628; International Application Publication No. WO1998043615, Calo, E. et al. (2015) Eur. Polymer J 65:252-267 and European Patent No. EP 058,481), including, for example, chemically synthesized polymers, starch based polymers, and polyhydroxyalkanoates (PHAs), copolymers of L-glutamic acid and gamma ethyl-L-glutamate (Sidman et al. (1993) Biopolymers 22:547-556), poly (2-hydroxyethyl-methacrylate) (Langer et al. (1981) J Biomed Mater Res. 15:167-277; and Langer (1982) Chem Tech 12:98-105), ethylene vinyl acetate (Hsu and Langer (1985) J Biomed Materials Res 19 (4): 445-460) or poly-D (-)-3-hydroxybutyric acid (European Patent No. EP0133988). In certain embodiments, sustained release compositions can also include liposomes, which can be prepared by any of several methods known in the art. (See, e.g., Eppstein et al. (1985) Proc. Natl. Acad. Sci. USA 82:3688-3692; European Patent No. EP 036,676; and U.S. Pat. Nos. 4,619,794 and 4,615,885).

The pharmaceutical composition to be used for in vivo administration typically is sterile. In certain embodiments, sterilization is accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

### 7. Unit Dose and Kits

In still another aspect, unit dose forms comprising two or more of the active ingredients as described in this disclosure are provided. A unit dose form can be formulated for administration according to any of the routes described in this disclosure. In one example, the unit dose form is formulated for intravenous or intraperitoneal administration. In still another aspect, pharmaceutical packages comprising unit dose forms of the active ingredients are provided. In some embodiments, unit dose forms are formulated for administration once daily (e.g., a capsule or pill). In some embodiments, unit dose forms are formulated for administration multiple times/day, e.g., 2 times/day, 3 times/day, or 4 times/day. In some embodiments, unit dose forms are formulated for administration from 1 time/year to 1 time/day, e.g., 1 time/year, 2 times/year, 4 times/year, 6 times/year, 12 times/year, 1 time/six months, 1 time/four months, 1 time/3 months, 1 time/2 months, 1 time/month, 2 times/month, 4 times/month, 8 times/month, 1 time/4 weeks, 1 time/3 weeks, 1 time/2 weeks, 1 time/week, 2 times/week, 3 times/week, 4 times/week, 5 times/week, 6 times/week, 7 times/week, or 1 time/day.

In some embodiments, the unit dose comprises at least one active ingredient component at a lower dose than the standard dose for said active ingredient. As used herein, the term “standard dose” refers to the dosage amount at which one of the active ingredients described above is generally prescribed for an approved therapeutic use. In some embodiments, the unit dose comprises an active ingredient at less than 100% of the standard dose, e.g., less than 99% of the standard dose, less than 98% of the standard dose, less than 96% of the standard dose, less than 94% of the standard dose, less than 92% of the standard dose, less than 90% of the standard dose, less than 85% of the standard dose, less than 80% of the standard dose, less than 75% of the standard dose, less than 70% of the standard dose, less than 65% of the standard dose, less than 60% of the standard dose, less than 55% of the standard dose, less than 50% of the standard dose, less than 45% of the standard dose, less than 40% of the standard dose, less than 35% of the standard dose, less than 30% of the standard dose, less than 25% of the standard dose, less than 20% of the standard dose, less than 15% of the standard dose, less than 10% of the standard dose, or less than 5% of the standard dose.

In some embodiments, the unit dose can comprise up to about 120 mg duloxetine or up to about 60 mg duloxetine, e.g., up to 58 mg duloxetine, up to 56 mg duloxetine, up to 54 mg duloxetine, up to 52 mg duloxetine, up to 50 mg duloxetine, up to 48 mg duloxetine, up to 46 mg duloxetine, up to 44 mg duloxetine, up to 42 mg duloxetine, up to 40 mg duloxetine, up to 38 mg duloxetine, up to 36 mg duloxetine, up to 34 mg duloxetine, up to 32 mg duloxetine, up to 30 mg duloxetine, up to 28 mg duloxetine, up to 26 mg duloxetine, up to 24 mg duloxetine, up to 22 mg duloxetine, up to 20 mg duloxetine, up to 18 mg duloxetine, up to 16 mg duloxetine, up to 14 mg duloxetine, up to 12 mg duloxetine, or up to 10 mg duloxetine. In terms of ranges, the unit dose can comprise from 10 mg to 60 mg duloxetine, e.g., from 15 mg to 55 mg duloxetine, from 20 mg to 50 mg duloxetine, from 25 mg to 45 mg duloxetine, from 30 mg to 40 mg duloxetine, from 10 mg to 30 mg duloxetine, from 10 mg to 20 mg duloxetine, from 30 mg to 45 mg duloxetine, from 30 mg to 50 mg duloxetine, from 30 mg to 60 mg duloxetine, from 40 mg to 60 mg duloxetine, or from 50 mg to 60 mg duloxetine. In some embodiments, the unit dose comprises one or more additional active ingredients in addition to duloxetine and the total active ingredients comprise from 0.1 wt. % to 99.9 wt. % duloxetine, e.g., up to 0.5 wt. % duloxetine, up to 1 wt. % duloxetine, up to 2 wt. % duloxetine, up to 4 wt. % duloxetine, up to 6 wt. % duloxetine, up to 8 wt. % duloxetine, up to 10 wt. % duloxetine, up to 15 wt. % duloxetine, up to 20 wt. % duloxetine, up to 25 wt. % duloxetine, up to 30 wt. % duloxetine, up to 35 wt. % duloxetine, up to 40 wt. % duloxetine, up to 45 wt. % duloxetine, up to 50 wt. % duloxetine, up to 55 wt. % duloxetine, up to 60 wt. % duloxetine, up to 65 wt. % duloxetine, up to 70 wt. % duloxetine, up to 75 wt. % duloxetine, up to 80 wt. % duloxetine, up to 85 wt. % duloxetine, up to 90 wt. % duloxetine, up to 92 wt. % duloxetine, up to 94 wt. % duloxetine, up to 96 wt. % duloxetine, up to 98 wt. % duloxetine, up to 99 wt. % duloxetine, or up to 99.5 wt. % duloxetine. In some embodiments, the unit dose comprising duloxetine is formulated for administration 1 time/day.

In some embodiments, the unit dose can comprise up to about 200 mg milnacipran, e.g., up to 195 mg milnacipran, up to 190 mg milnacipran, up to 185 mg milnacipran, up to 180 mg milnacipran, up to 175 mg milnacipran, up to 170 mg milnacipran, up to 165 mg milnacipran, up to 160 mg milnacipran, up to 155 mg milnacipran, up to 150 mg milnacipran, up to 145 mg milnacipran, up to 140 mg milnacipran, up to 135 mg milnacipran, up to 130 mg milnacipran, up to 125 mg milnacipran, up to 120 mg milnacipran, up to 115 mg milnacipran, up to 110 mg milnacipran, up to 105 mg milnacipran, up to 100 mg milnacipran, up to 95 mg milnacipran, up to 90 mg milnacipran, up to 85 mg milnacipran, up to 80 mg milnacipran, up to 75 mg milnacipran, up to 70 mg milnacipran, up to 65 mg milnacipran, up to 60 mg milnacipran, up to 55 mg milnacipran, up to 50 mg milnacipran, up to 45 mg milnacipran, up to 40 mg milnacipran, up to 35 mg milnacipran, up to 30 mg milnacipran, up to 25 mg milnacipran, up to 20 mg milnacipran, up to 15 mg milnacipran, or up to 10 mg milnacipran. In terms of ranges, the unit dose can comprise from 10 mg to 200 mg milnacipran, e.g., from 20 mg to 190 mg milnacipran, from 30 mg to 180 mg milnacipran, from 40 mg to 170 mg milnacipran, from 50 mg to 160 mg milnacipran, from 60 mg to 150 mg milnacipran, from 70 mg to 140 mg milnacipran, from 80 mg to 130 mg milnacipran, from 90 mg to 120 mg milnacipran, from 100 mg to 110 mg milnacipran, from 10 mg to 100 mg milnacipran, from 15 mg to 90 mg milnacipran, from 20 mg to 80 mg milnacipran, from 25 mg to 75 mg milnacipran, from 100 mg to 200 mg milnacipran, from 110 mg to 190 mg milnacipran, from 120 mg to 180 mg milnacipran, from 125 mg to 175 mg milnacipran, from 130 mg to 165 mg milnacipran, from 50 mg to 150 mg milnacipran, from 60 mg to 150 mg milnacipran, from 75 mg to 150 mg milnacipran, or from 75 mg to 125 mg milnacipran. In some embodiments, the unit dose comprises one or more additional active ingredients in addition to milnacipran and the total active ingredients comprise from 0.1 wt. % to 99.9 wt. % milnacipran, e.g., up to 0.5 wt. % milnacipran, up to 1 wt. % milnacipran, up to 2 wt. % milnacipran, up to 4 wt. % milnacipran, up to 6 wt. % milnacipran, up to 8 wt. % milnacipran, up to 10 wt. % milnacipran, up to 15 wt. % milnacipran, up to 20 wt. % milnacipran, up to 25 wt. % milnacipran, up to 30 wt. % milnacipran, up to 35 wt. % milnacipran, up to 40 wt. % milnacipran, up to 45 wt. % milnacipran, up to 50 wt. % milnacipran, up to 55 wt. % milnacipran, up to 60 wt. % milnacipran, up to 65 wt. % milnacipran, up to 70 wt. % milnacipran, up to 75 wt. % milnacipran, up to 80 wt. % milnacipran, up to 85 wt. % milnacipran, up to 90 wt. % milnacipran, up to 92 wt. % milnacipran, up to 94 wt. % milnacipran, up to 96 wt. % milnacipran, up to 98 wt. % milnacipran, up to 99 wt. % milnacipran, or up to 99.5 wt. % milnacipran. In some embodiments, the unit dose comprising milnacipran is formulated for administration 1 time/day.

In some embodiments, the unit dose can comprise up to about 75 mg rimegepant, e.g., up to 74 mg rimegepant, up to 73 mg rimegepant, up to 72 mg rimegepant, up to 70 mg rimegepant, up to 68 mg rimegepant, up to 66 mg rimegepant, up to 64 mg rimegepant, up to 62 mg rimegepant, up to 60 mg rimegepant, up to 58 mg rimegepant, up to 56 mg rimegepant, up to 54 mg rimegepant, up to 52 mg rimegepant, up to 50 mg rimegepant, up to 48 mg rimegepant, up to 46 mg rimegepant, up to 44 mg rimegepant, up to 42 mg rimegepant, up to 40 mg rimegepant, up to 38 mg rimegepant, up to 36 mg rimegepant, up to 34 mg rimegepant, up to 32 mg rimegepant, up to 30 mg rimegepant, up to 28 mg rimegepant, up to 26 mg rimegepant, up to 24 mg rimegepant, up to 22 mg rimegepant, up to 20 mg rimegepant, up to 18 mg rimegepant, up to 16 mg rimegepant, up to 14 mg rimegepant, up to 12 mg rimegepant, or up to 10 mg rimegepant. In terms of ranges, the unit dose can comprise from 10 mg to 75 mg rimegepant, e.g., from 15 mg to 70 mg rimegepant, from 20 mg to 65 mg rimegepant, from 25 mg to 60 mg rimegepant, from 30 mg to 55 mg rimegepant, from 10 mg to 40 mg rimegepant, from 10 mg to 30 mg rimegepant, from 10 mg to 20 mg rimegepant, from 30 mg to 45 mg rimegepant, from 30 mg to 50 mg rimegepant, from 30 mg to 60 mg rimegepant, from 30 mg to 70 mg rimegepant, from 30 mg to 75 mg rimegepant, from 40 mg to 75 mg rimegepant, from 50 mg to 75 mg rimegepant, or from 60 mg to 75 mg rimegepant. In some embodiments, the unit dose comprises one or more additional active ingredients in addition to rimegepant and the total active ingredients comprise from 0.1 wt. % to 99.9 wt. % rimegepant, e.g., up to 0.5 wt. % rimegepant, up to 1 wt. % rimegepant, up to 2 wt. % rimegepant, up to 4 wt. % rimegepant, up to 6 wt. % rimegepant, up to 8 wt. % rimegepant, up to 10 wt. % rimegepant, up to 15 wt. % rimegepant, up to 20 wt. % rimegepant, up to 25 wt. % rimegepant, up to 30 wt. % rimegepant, up to 35 wt. % rimegepant, up to 40 wt. % rimegepant, up to 45 wt. % rimegepant, up to 50 wt. % rimegepant, up to 55 wt. % rimegepant, up to 60 wt. % rimegepant, up to 65 wt. % rimegepant, up to 70 wt. % rimegepant, up to 75 wt. % rimegepant, up to 80 wt. % rimegepant, up to 85 wt. % rimegepant, up to 90 wt. % rimegepant, up to 92 wt. % rimegepant, up to 94 wt. % rimegepant, up to 96 wt. % rimegepant, up to 98 wt. % rimegepant, up to 99 wt. % rimegepant, or up to 99.5 wt. % rimegepant. In some embodiments, the unit dose comprising rimegepant is formulated for administration 1 time/day.

In some embodiments, the unit dose can comprise up to about 200 mg ubrogepant, e.g., up to 195 mg ubrogepant, up to 190 mg ubrogepant, up to 185 mg ubrogepant, up to 180 mg ubrogepant, up to 175 mg ubrogepant, up to 170 mg ubrogepant, up to 165 mg ubrogepant, up to 160 mg ubrogepant, up to 155 mg ubrogepant, up to 150 mg ubrogepant, up to 145 mg ubrogepant, up to 140 mg ubrogepant, up to 135 mg ubrogepant, up to 130 mg ubrogepant, up to 125 mg ubrogepant, up to 120 mg ubrogepant, up to 115 mg ubrogepant, up to 110 mg ubrogepant, up to 105 mg ubrogepant, up to 100 mg ubrogepant, up to 95 mg ubrogepant, up to 90 mg ubrogepant, up to 85 mg ubrogepant, up to 80 mg ubrogepant, up to 75 mg ubrogepant, up to 70 mg ubrogepant, up to 65 mg ubrogepant, up to 60 mg ubrogepant, up to 55 mg ubrogepant, up to 50 mg ubrogepant, up to 45 mg ubrogepant, up to 40 mg ubrogepant, up to 35 mg ubrogepant, up to 30 mg ubrogepant, up to 25 mg ubrogepant, up to 20 mg ubrogepant, up to 15 mg ubrogepant, or up to 10 mg ubrogepant. In terms of ranges, the unit dose can comprise from 10 mg to 200 mg ubrogepant, e.g., from 20 mg to 190 mg ubrogepant, from 30 mg to 180 mg ubrogepant, from 40 mg to 170 mg ubrogepant, from 50 mg to 160 mg ubrogepant, from 60 mg to 150 mg ubrogepant, from 70 mg to 140 mg ubrogepant, from 80 mg to 130 mg ubrogepant, from 90 mg to 120 mg ubrogepant, from 100 mg to 110 mg ubrogepant, from 10 mg to 30 mg ubrogepant, from 10 mg to 40 mg ubrogepant, from 10 mg to 45 mg ubrogepant, from 10 mg to 50 mg ubrogepant, from 10 mg to 75 mg ubrogepant, from 10 mg to 100 mg ubrogepant, from 15 mg to 90 mg ubrogepant, from 20 mg to 80 mg ubrogepant, from 25 mg to 75 mg ubrogepant, from 100 mg to 200 mg ubrogepant, from 110 mg to 190 mg ubrogepant, from 120 mg to 180 mg ubrogepant, from 125 mg to 175 mg ubrogepant, from 130 mg to 165 mg ubrogepant, from 50 mg to 150 mg ubrogepant, from 60 mg to 150 mg ubrogepant, from 75 mg to 150 mg ubrogepant, or from 75 mg to 125 mg ubrogepant. In some embodiments, the unit dose comprises one or more additional active ingredients in addition to ubrogepant and the total active ingredients comprise from 0.1 wt. % to 99.9 wt. % ubrogepant, e.g., up to 0.5 wt. % ubrogepant, up to 1 wt. % ubrogepant, up to 2 wt. % ubrogepant, up to 4 wt. % ubrogepant, up to 6 wt. % ubrogepant, up to 8 wt. % ubrogepant, up to 10 wt. % ubrogepant, up to 15 wt. % ubrogepant, up to 20 wt. % ubrogepant, up to 25 wt. % ubrogepant, up to 30 wt. % ubrogepant, up to 35 wt. % ubrogepant, up to 40 wt. % ubrogepant, up to 45 wt. % ubrogepant, up to 50 wt. % ubrogepant, up to 55 wt. % ubrogepant, up to 60 wt. % ubrogepant, up to 65 wt. % ubrogepant, up to 70 wt. % ubrogepant, up to 75 wt. % ubrogepant, up to 80 wt. % ubrogepant, up to 85 wt. % ubrogepant, up to 90 wt. % ubrogepant, up to 92 wt. % ubrogepant, up to 94 wt. % ubrogepant, up to 96 wt. % ubrogepant, up to 98 wt. % ubrogepant, up to 99 wt. % ubrogepant, or up to 99.5 wt. % ubrogepant. In some embodiments, the unit dose comprising ubrogepant is formulated for administration 1 time/day.

In some embodiments, the unit dose can comprise up to about 60 mg atogepant, e.g., up to 58 mg atogepant, up to 56 mg atogepant, up to 54 mg atogepant, up to 52 mg atogepant, up to 50 mg atogepant, up to 48 mg atogepant, up to 46 mg atogepant, up to 44 mg atogepant, up to 42 mg atogepant, up to 40 mg atogepant, up to 38 mg atogepant, up to 36 mg atogepant, up to 34 mg atogepant, up to 32 mg atogepant, up to 30 mg atogepant, up to 28 mg atogepant, up to 26 mg atogepant, up to 24 mg atogepant, up to 22 mg atogepant, up to 20 mg atogepant, up to 18 mg atogepant, up to 16 mg atogepant, up to 14 mg atogepant, up to 12 mg atogepant, or up to 10 mg atogepant. In terms of ranges, the unit dose can comprise from 10 mg to 60 mg atogepant, e.g., from 15 mg to 55 mg atogepant, from 20 mg to 50 mg atogepant, from 25 mg to 45 mg atogepant, from 30 mg to 40 mg atogepant, from 10 mg to 30 mg atogepant, from 10 mg to 20 mg atogepant, from 30 mg to 45 mg atogepant, from 30 mg to 50 mg atogepant, from 30 mg to 60 mg atogepant, from 40 mg to 60 mg atogepant, or from 50 mg to 60 mg atogepant. In some embodiments, the unit dose comprises one or more additional active ingredients in addition to atogepant and the total active ingredients comprise from 0.1 wt. % to 99.9 wt. % atogepant, e.g., up to 0.5 wt. % atogepant, up to 1 wt. % atogepant, up to 2 wt. % atogepant, up to 4 wt. % atogepant, up to 6 wt. % atogepant, up to 8 wt. % atogepant, up to 10 wt. % atogepant, up to 15 wt. % atogepant, up to 20 wt. % atogepant, up to 25 wt. % atogepant, up to 30 wt. % atogepant, up to 35 wt. % atogepant, up to 40 wt. % atogepant, up to 45 wt. % atogepant, up to 50 wt. % atogepant, up to 55 wt. % atogepant, up to 60 wt. % atogepant, up to 65 wt. % atogepant, up to 70 wt. % atogepant, up to 75 wt. % atogepant, up to 80 wt. % atogepant, up to 85 wt. % atogepant, up to 90 wt. % atogepant, up to 92 wt. % atogepant, up to 94 wt. % atogepant, up to 96 wt. % atogepant, up to 98 wt. % atogepant, up to 99 wt. % atogepant, or up to 99.5 wt. % atogepant. In some embodiments, the unit dose comprising atogepant is formulated for administration 1 time/day.

In some embodiments, the unit dose can comprise up to about 140 mg erenumab, e.g., up to 139 mg erenumab, up to 138 mg erenumab, up to 137 mg erenumab, up to 135 mg erenumab, up to 130 mg erenumab, up to 125 mg erenumab, up to 120 mg erenumab, up to 115 mg erenumab, up to 110 mg erenumab, up to 105 mg erenumab, up to 100 mg erenumab, up to 95 mg erenumab, up to 90 mg erenumab, up to 85 mg erenumab, up to 80 mg erenumab, up to 75 mg erenumab, up to 70 mg erenumab, up to 65 mg erenumab, up to 60 mg erenumab, up to 55 mg erenumab, up to 50 mg erenumab, up to 45 mg erenumab, up to 40 mg erenumab, up to 35 mg erenumab, up to 30 mg erenumab, up to 25 mg erenumab, up to 20 mg erenumab, up to 15 mg erenumab, or up to 10 mg erenumab. In terms of ranges, the unit dose can comprise from 10 mg to 140 mg erenumab, e.g., from 15 mg to 135 mg erenumab, from 20 mg to 130 mg erenumab, from 25 mg to 125 mg erenumab, from 30 mg to 120 mg erenumab, from 35 mg to 115 mg erenumab, from 40 mg to 110 mg erenumab, from 45 mg to 105 mg erenumab, from 50 mg to 100 mg erenumab, from 55 mg to 95 mg erenumab, from 60 mg to 90 mg erenumab, from 10 mg to 30 mg erenumab, from 10 mg to 40 mg erenumab, from 10 mg to 45 mg erenumab, from 10 mg to 50 mg erenumab, from 10 mg to 60 mg erenumab, from 10 mg to 70 mg erenumab, from 10 mg to 75 mg erenumab, from 10 mg to 100 mg erenumab, from 15 mg to 90 mg erenumab, from 20 mg to 80 mg erenumab, from 25 mg to 75 mg erenumab, from 70 mg to 120 mg erenumab, from 70 mg to 125 mg erenumab, from 70 mg to 130 mg erenumab, from 70 mg to 135 mg erenumab, or from 70 mg to 140 mg erenumab. In some embodiments, the unit dose comprises one or more additional active ingredients in addition to erenumab and the total active ingredients comprise from 0.1 wt. % to 99.9 wt. % erenumab, e.g., up to 0.5 wt. % erenumab, up to 1 wt. % erenumab, up to 2 wt. % erenumab, up to 4 wt. % erenumab, up to 6 wt. % erenumab, up to 8 wt. % erenumab, up to 10 wt. % erenumab, up to 15 wt. % erenumab, up to 20 wt. % erenumab, up to 25 wt. % erenumab, up to 30 wt. % erenumab, up to 35 wt. % erenumab, up to 40 wt. % erenumab, up to 45 wt. % erenumab, up to 50 wt. % erenumab, up to 55 wt. % erenumab, up to 60 wt. % erenumab, up to 65 wt. % erenumab, up to 70 wt. % erenumab, up to 75 wt. % erenumab, up to 80 wt. % erenumab, up to 85 wt. % erenumab, up to 90 wt. % erenumab, up to 92 wt. % erenumab, up to 94 wt. % erenumab, up to 96 wt. % erenumab, up to 98 wt. % erenumab, up to 99 wt. % erenumab, or up to 99.5 wt. % erenumab. In some embodiments, the unit dose comprising erenumab is formulated for administration 1 time/month.

In some embodiments, the unit dose can comprise up to about 300 mg eptinezumab, e.g., up to 295 mg eptinezumab, up to 290 mg eptinezumab, up to 285 mg eptinezumab, up to 280 mg eptinezumab, up to 275 mg eptinezumab, up to 270 mg eptinezumab, up to 265 mg eptinezumab, up to 260 mg eptinezumab, up to 255 mg eptinezumab, up to 250 mg eptinezumab, up to 245 mg eptinezumab, up to 240 mg eptinezumab, up to 235 mg eptinezumab, up to 230 mg eptinezumab, up to 225 mg eptinezumab, up to 220 mg eptinezumab, up to 215 mg eptinezumab, up to 210 mg eptinezumab, up to 205 mg eptinezumab, up to 200 mg eptinezumab, up to 195 mg eptinezumab, up to 190 mg eptinezumab, up to 185 mg eptinezumab, up to 180 mg eptinezumab, up to 175 mg eptinezumab, up to 170 mg eptinezumab, up to 165 mg eptinezumab, up to 160 mg eptinezumab, up to 155 mg eptinezumab, up to 150 mg eptinezumab, up to 145 mg eptinezumab, up to 140 mg eptinezumab, up to 135 mg eptinezumab, up to 130 mg eptinezumab, up to 125 mg eptinezumab, up to 120 mg eptinezumab, up to 115 mg eptinezumab, up to 110 mg eptinezumab, up to 105 mg eptinezumab, up to 100 mg eptinezumab, up to 95 mg eptinezumab, up to 90 mg eptinezumab, up to 85 mg eptinezumab, up to 80 mg eptinezumab, up to 75 mg eptinezumab, up to 70 mg eptinezumab, up to 65 mg eptinezumab, up to 60 mg eptinezumab, up to 55 mg eptinezumab, up to 50 mg eptinezumab, up to 45 mg eptinezumab, up to 40 mg eptinezumab, up to 35 mg eptinezumab, up to 30 mg eptinezumab, up to 25 mg eptinezumab, up to 20 mg eptinezumab, up to 15 mg eptinezumab, or up to 10 mg eptinezumab. In terms of ranges, the unit dose can comprise from 10 mg to 300 mg eptinezumab, e.g., from 20 mg to 290 mg eptinezumab, from 30 mg to 280 mg eptinezumab, from 40 mg to 270 mg eptinezumab, from 50 mg to 260 mg eptinezumab, from 60 mg to 250 mg eptinezumab, from 70 mg to 240 mg eptinezumab, from 80 mg to 230 mg eptinezumab, from 90 mg to 220 mg eptinezumab, from 100 mg to 210 mg eptinezumab, from 110 mg to 200 mg eptinezumab, from 120 mg to 190 mg eptinezumab, from 130 mg to 180 mg eptinezumab, from 140 mg to 170 mg eptinezumab, from 10 mg to 30 mg eptinezumab, from 10 mg to 40 mg eptinezumab, from 10 mg to 45 mg eptinezumab, from 10 mg to 50 mg eptinezumab, from 10 mg to 75 mg eptinezumab, from 10 mg to 100 mg eptinezumab, from 10 mg to 150 mg eptinezumab, from 100 mg to 150 mg eptinezumab, from 100 mg to 175 mg eptinezumab, from 100 mg to 200 mg eptinezumab, from 150 mg to 300 mg eptinezumab, from 150 mg to 275 mg eptinezumab, from 150 mg to 250 mg eptinezumab, from 150 mg to 225 mg eptinezumab, from 150 mg to 200 mg eptinezumab, from 200 mg to 300 mg eptinezumab, from 200 mg to 275 mg eptinezumab, or from 200 mg to 250 mg eptinezumab. In some embodiments, the unit dose comprises one or more additional active ingredients in addition to eptinezumab and the total active ingredients comprise from 0.1 wt. % to 99.9 wt. % eptinezumab, e.g., up to 0.5 wt. % eptinezumab, up to 1 wt. % eptinezumab, up to 2 wt. % eptinezumab, up to 4 wt. % eptinezumab, up to 6 wt. % eptinezumab, up to 8 wt. % eptinezumab, up to 10 wt. % eptinezumab, up to 15 wt. % eptinezumab, up to 20 wt. % eptinezumab, up to 25 wt. % eptinezumab, up to 30 wt. % eptinezumab, up to 35 wt. % eptinezumab, up to 40 wt. % eptinezumab, up to 45 wt. % eptinezumab, up to 50 wt. % eptinezumab, up to 55 wt. % eptinezumab, up to 60 wt. % eptinezumab, up to 65 wt. % eptinezumab, up to 70 wt. % eptinezumab, up to 75 wt. % eptinezumab, up to 80 wt. % eptinezumab, up to 85 wt. % eptinezumab, up to 90 wt. % eptinezumab, up to 92 wt. % eptinezumab, up to 94 wt. % eptinezumab, up to 96 wt. % eptinezumab, up to 98 wt. % eptinezumab, up to 99 wt. % eptinezumab, or up to 99.5 wt. % eptinezumab. In some embodiments, the unit dose comprising eptinezumab is formulated for administration 1 time/month. In some embodiments, the unit dose comprising eptinezumab is formulated for administration 1 time/3 months.

In some embodiments, the unit dose can comprise up to about 225 mg fremanezumab, e.g., up to 220 mg fremanezumab, up to 215 mg fremanezumab, up to 210 mg fremanezumab, up to 205 mg fremanezumab, up to 200 mg fremanezumab, up to 195 mg fremanezumab, up to 190 mg fremanezumab, up to 185 mg fremanezumab, up to 180 mg fremanezumab, up to 175 mg fremanezumab, up to 170 mg fremanezumab, up to 165 mg fremanezumab, up to 160 mg fremanezumab, up to 155 mg fremanezumab, up to 150 mg fremanezumab, up to 145 mg fremanezumab, up to 140 mg fremanezumab, up to 135 mg fremanezumab, up to 130 mg fremanezumab, up to 125 mg fremanezumab, up to 120 mg fremanezumab, up to 115 mg fremanezumab, up to 110 mg fremanezumab, up to 105 mg fremanezumab, up to 100 mg fremanezumab, up to 95 mg fremanezumab, up to 90 mg fremanezumab, up to 85 mg fremanezumab, up to 80 mg fremanezumab, up to 75 mg fremanezumab, up to 70 mg fremanezumab, up to 65 mg fremanezumab, up to 60 mg fremanezumab, up to 55 mg fremanezumab, up to 50 mg fremanezumab, up to 45 mg fremanezumab, up to 40 mg fremanezumab, up to 35 mg fremanezumab, up to 30 mg fremanezumab, up to 25 mg fremanezumab, up to 20 mg fremanezumab, up to 15 mg fremanezumab, or up to 10 mg fremanezumab. In terms of ranges, the unit dose can comprise from 10 mg to 225 mg fremanezumab, e.g., from 20 mg to 220 mg fremanezumab, from 30 mg to 215 mg fremanezumab, from 40 mg to 210 mg fremanezumab, from 50 mg to 205 mg fremanezumab, from 60 mg to 200 mg fremanezumab, from 70 mg to 190 mg fremanezumab, from 80 mg to 180 mg fremanezumab, from 90 mg to 170 mg fremanezumab, from 100 mg to 160 mg fremanezumab, from 10 mg to 30 mg fremanezumab, from 10 mg to 40 mg fremanezumab, from 10 mg to 45 mg fremanezumab, from 10 mg to 50 mg fremanezumab, from 10 mg to 75 mg fremanezumab, from 10 mg to 100 mg fremanezumab, from 10 mg to 115 mg fremanezumab, from 10 mg to 125 mg fremanezumab, from 15 mg to 90 mg fremanezumab, from 20 mg to 80 mg fremanezumab, from 25 mg to 75 mg fremanezumab, from 100 mg to 225 mg fremanezumab, from 110 mg to 220 mg fremanezumab, from 120 mg to 210 mg fremanezumab, from 125 mg to 175 mg fremanezumab, from 130 mg to 165 mg fremanezumab, from 50 mg to 150 mg fremanezumab, from 60 mg to 150 mg fremanezumab, from 75 mg to 150 mg fremanezumab, from 75 mg to 125 mg fremanezumab, from 150 mg to 225 mg fremanezumab, from 160 mg to 225 mg fremanezumab, from 175 mg to 225 mg fremanezumab, or from 200 mg to 225 mg fremanezumab. In some embodiments, the unit dose comprises one or more additional active ingredients in addition to fremanezumab and the total active ingredients comprise from 0.1 wt. % to 99.9 wt. % fremanezumab, e.g., up to 0.5 wt. % fremanezumab, up to 1 wt. % fremanezumab, up to 2 wt. % fremanezumab, up to 4 wt. % fremanezumab, up to 6 wt. % fremanezumab, up to 8 wt. % fremanezumab, up to 10 wt. % fremanezumab, up to 15 wt. % fremanezumab, up to 20 wt. % fremanezumab, up to 25 wt. % fremanezumab, up to 30 wt. % fremanezumab, up to 35 wt. % fremanezumab, up to 40 wt. % fremanezumab, up to 45 wt. % fremanezumab, up to 50 wt. % fremanezumab, up to 55 wt. % fremanezumab, up to 60 wt. % fremanezumab, up to 65 wt. % fremanezumab, up to 70 wt. % fremanezumab, up to 75 wt. % fremanezumab, up to 80 wt. % fremanezumab, up to 85 wt. % fremanezumab, up to 90 wt. % fremanezumab, up to 92 wt. % fremanezumab, up to 94 wt. % fremanezumab, up to 96 wt. % fremanezumab, up to 98 wt. % fremanezumab, up to 99 wt. % fremanezumab, or up to 99.5 wt. % fremanezumab. In some embodiments, the unit dose comprising fremanezumab is formulated for administration 1 time/month.

In some embodiments, the unit dose can comprise up to about 300 mg galcanezumab, e.g., up to 295 mg galcanezumab, up to 290 mg galcanezumab, up to 285 mg galcanezumab, up to 280 mg galcanezumab, up to 275 mg galcanezumab, up to 270 mg galcanezumab, up to 265 mg galcanezumab, up to 260 mg galcanezumab, up to 255 mg galcanezumab, up to 250 mg galcanezumab, up to 245 mg galcanezumab, up to 240 mg galcanezumab, up to 235 mg galcanezumab, up to 230 mg galcanezumab, up to 225 mg galcanezumab, up to 220 mg galcanezumab, up to 215 mg galcanezumab, up to 210 mg galcanezumab, up to 205 mg galcanezumab, up to 200 mg galcanezumab, up to 195 mg galcanezumab, up to 190 mg galcanezumab, up to 185 mg galcanezumab, up to 180 mg galcanezumab, up to 175 mg galcanezumab, up to 170 mg galcanezumab, up to 165 mg galcanezumab, up to 160 mg galcanezumab, up to 155 mg galcanezumab, up to 150 mg galcanezumab, up to 145 mg galcanezumab, up to 140 mg galcanezumab, up to 135 mg galcanezumab, up to 130 mg galcanezumab, up to 125 mg galcanezumab, up to 120 mg galcanezumab, up to 115 mg galcanezumab, up to 110 mg galcanezumab, up to 105 mg galcanezumab, up to 100 mg galcanezumab, up to 95 mg galcanezumab, up to 90 mg galcanezumab, up to 85 mg galcanezumab, up to 80 mg galcanezumab, up to 75 mg galcanezumab, up to 70 mg galcanezumab, up to 65 mg galcanezumab, up to 60 mg galcanezumab, up to 55 mg galcanezumab, up to 50 mg galcanezumab, up to 45 mg galcanezumab, up to 40 mg galcanezumab, up to 35 mg galcanezumab, up to 30 mg galcanezumab, up to 25 mg galcanezumab, up to 20 mg galcanezumab, up to 15 mg galcanezumab, or up to 10 mg galcanezumab. In terms of ranges, the unit dose can comprise from 10 mg to 300 mg galcanezumab, e.g., from 20 mg to 290 mg galcanezumab, from 30 mg to 280 mg galcanezumab, from 40 mg to 270 mg galcanezumab, from 50 mg to 260 mg galcanezumab, from 60 mg to 250 mg galcanezumab, from 70 mg to 240 mg galcanezumab, from 80 mg to 230 mg galcanezumab, from 90 mg to 220 mg galcanezumab, from 100 mg to 210 mg galcanezumab, from 110 mg to 200 mg galcanezumab, from 120 mg to 190 mg galcanezumab, from 130 mg to 180 mg galcanezumab, from 140 mg to 170 mg galcanezumab, from 10 mg to 30 mg galcanezumab, from 10 mg to 40 mg galcanezumab, from 10 mg to 45 mg galcanezumab, from 10 mg to 50 mg galcanezumab, from 10 mg to 75 mg galcanezumab, from 10 mg to 100 mg galcanezumab, from 10 mg to 150 mg galcanezumab, from 100 mg to 150 mg galcanezumab, from 100 mg to 175 mg galcanezumab, from 100 mg to 200 mg galcanezumab, from 150 mg to 300 mg galcanezumab, from 150 mg to 275 mg galcanezumab, from 150 mg to 250 mg galcanezumab, from 150 mg to 225 mg galcanezumab, from 150 mg to 200 mg galcanezumab, from 200 mg to 300 mg galcanezumab, from 200 mg to 275 mg galcanezumab, or from 200 mg to 250 mg galcanezumab. In some embodiments, the unit dose comprises one or more additional active ingredients in addition to galcanezumab and the total active ingredients comprise from 0.1 wt. % to 99.9 wt. % galcanezumab, e.g., up to 0.5 wt. % galcanezumab, up to 1 wt. % galcanezumab, up to 2 wt. % galcanezumab, up to 4 wt. % galcanezumab, up to 6 wt. % galcanezumab, up to 8 wt. % galcanezumab, up to 10 wt. % galcanezumab, up to 15 wt. % galcanezumab, up to 20 wt. % galcanezumab, up to 25 wt. % galcanezumab, up to 30 wt. % galcanezumab, up to 35 wt. % galcanezumab, up to 40 wt. % galcanezumab, up to 45 wt. % galcanezumab, up to 50 wt. % galcanezumab, up to 55 wt. % galcanezumab, up to 60 wt. % galcanezumab, up to 65 wt. % galcanezumab, up to 70 wt. % galcanezumab, up to 75 wt. % galcanezumab, up to 80 wt. % galcanezumab, up to 85 wt. % galcanezumab, up to 90 wt. % galcanezumab, up to 92 wt. % galcanezumab, up to 94 wt. % galcanezumab, up to 96 wt. % galcanezumab, up to 98 wt. % galcanezumab, up to 99 wt. % galcanezumab, or up to 99.5 wt. % galcanezumab. In some embodiments, the unit dose comprising galcanezumab is formulated for administration 1 time/month.

In some embodiments, the unit dose can comprise up to about 0.6 mg clonidine, e.g., up to 0.58 mg clonidine, up to 0.56 mg clonidine, up to 0.54 mg clonidine, up to 0.52 mg clonidine, up to 0.50 mg clonidine, up to 0.48 mg clonidine, up to 0.46 mg clonidine, up to 0.44 mg clonidine, up to 0.42 mg clonidine, up to 0.40 mg clonidine, up to 0.38 mg clonidine, up to 0.36 mg clonidine, up to 0.34 mg clonidine, up to 0.32 mg clonidine, up to 0.30 mg clonidine, up to 0.28 mg clonidine, up to 0.26 mg clonidine, up to 0.24 mg clonidine, up to 0.22 mg clonidine, up to 0.20 mg clonidine, up to 0.18 mg clonidine, up to 0.16 mg clonidine, up to 0.14 mg clonidine, up to 0.12 mg clonidine, up to 0.10 mg clonidine, up to 0.08 mg clonidine, up to 0.06 mg clonidine, up to 0.04 mg clonidine, up to 0.02 mg clonidine, or up to 0.01 mg clonidine. In terms of ranges, the unit dose can comprise from 0.01 mg to 0.60 mg clonidine, e.g., from 0.02 mg to 0.55 mg clonidine, from 0.05 mg to 0.50 mg clonidine, from 0.1 mg to 0.50 mg clonidine, from 0.20 mg to 0.40 mg clonidine, from 0.01 mg to 0.30 mg clonidine, from 0.01 mg to 0.20 mg clonidine, from 0.01 mg to 0.10 mg clonidine, from 0.10 mg to 0.40 mg clonidine, from 0.30 mg to 0.60 mg clonidine, from 0.40 mg to 0.60 mg clonidine, or from 0.50 mg to 0.60 mg clonidine. In some embodiments, the unit dose comprises one or more additional active ingredients in addition to clonidine and the total active ingredients comprise from 0.1 wt. % to 99.9 wt. % clonidine, e.g., up to 0.5 wt. % clonidine, up to 1 wt. % clonidine, up to 2 wt. % clonidine, up to 4 wt. % clonidine, up to 6 wt. % clonidine, up to 8 wt. % clonidine, up to 10 wt. % clonidine, up to 15 wt. % clonidine, up to 20 wt. % clonidine, up to 25 wt. % clonidine, up to 30 wt. % clonidine, up to 35 wt. % clonidine, up to 40 wt. % clonidine, up to 45 wt. % clonidine, up to 50 wt. % clonidine, up to 55 wt. % clonidine, up to 60 wt. % clonidine, up to 65 wt. % clonidine, up to 70 wt. % clonidine, up to 75 wt. % clonidine, up to 80 wt. % clonidine, up to 85 wt. % clonidine, up to 90 wt. % clonidine, up to 92 wt. % clonidine, up to 94 wt. % clonidine, up to 96 wt. % clonidine, up to 98 wt. % clonidine, up to 99 wt. % clonidine, or up to 99.5 wt. % clonidine. In some embodiments, the unit dose comprising clonidine is formulated for administration 1 time/day.

In some embodiments, the unit dose can comprise an SNRI at up to about 300 mg, e.g., up to 295 mg, up to 290 mg, up to 285 mg, up to 280 mg, up to 275 mg, up to 270 mg, up to 265 mg, up to 260 mg, up to 255 mg, up to 250 mg, up to 245 mg, up to 240 mg, up to 235 mg, up to 230 mg, up to 225 mg, up to 220 mg, up to 215 mg, up to 210 mg, up to 205 mg, up to 200 mg, up to 195 mg, up to 190 mg, up to 185 mg, up to 180 mg, up to 175 mg, up to 170 mg, up to 165 mg, up to 160 mg, up to 155 mg, up to 150 mg, up to 145 mg, up to 140 mg, up to 135 mg, up to 130 mg, up to 125 mg, up to 120 mg, up to 115 mg, up to 110 mg, up to 105 mg, up to 100 mg, up to 95 mg, up to 90 mg, up to 85 mg, up to 80 mg, up to 75 mg, up to 70 mg, up to 65 mg, up to 60 mg, up to 55 mg, up to 50 mg, up to 45 mg, up to 40 mg, up to 35 mg, up to 30 mg, up to 25 mg, up to 20 mg, up to 15 mg, up to 10 mg, up to 5 mg, up to 2 mg, up to 1 mg, up to 0.7 mg, up to 0.6 mg, up to 0.5 mg, up to 0.4 mg, up to 0.3 mg, up to 0.2 mg, or up to 0.1 mg. In terms of ranges, the unit dose can comprise an SNRI from 0.1 mg to 300 mg, e.g., from 0.1 mg to 295 mg, from 0.5 mg to 295 mg, from 1 mg to 290 mg, from 5 mg to 290 mg, from 10 mg to 285 mg, from 20 mg to 285 mg, from 30 mg to 280 mg, from 40 mg to 270 mg, from 50 mg to 260 mg, from 60 mg to 250 mg, from 70 mg to 240 mg, from 80 mg to 230 mg, from 90 mg to 220 mg, from 100 mg to 210 mg, from 110 mg to 200 mg, from 120 mg to 190 mg, from 130 mg to 180 mg, from 140 mg to 170 mg, from 0.1 mg to 30 mg, from 0.1 mg to 40 mg, from 0.1 mg to 45 mg, from 0.1 mg to 50 mg, from 0.1 mg to 75 mg, from 0.1 mg to 100 mg, from 0.1 mg to 150 mg, from 100 mg to 150 mg, from 100 mg to 175 mg, from 100 mg to 200 mg, from 150 mg to 300 mg, from 150 mg to 275 mg, from 150 mg to 250 mg, from 150 mg to 225 mg, from 150 mg to 200 mg, from 200 mg to 300 mg, from 200 mg to 275 mg, or from 200 mg to 250 mg. In some embodiments, the unit comprises one or more additional active ingredients in addition to an SNRI, and the total active ingredients in the unit dose comprise from 0.1 wt. % to 99.9 wt. % SNRI, e.g., up to 0.5 wt. % SNRI, up to 1 wt. % SNRI, up to 2 wt. % SNRI, up to 4 wt. % SNRI, up to 6 wt. % SNRI, up to 8 wt. % SNRI, up to 10 wt. % SNRI, up to 15 wt. % SNRI, up to 20 wt. % SNRI, up to 25 wt. % SNRI, up to 30 wt. % SNRI, up to 35 wt. % SNRI, up to 40 wt. % SNRI, up to 45 wt. % SNRI, up to 50 wt. % SNRI, up to 55 wt. % SNRI, up to 60 wt. % SNRI, up to 65 wt. % SNRI, up to 70 wt. % SNRI, up to 75 wt. % SNRI, up to 80 wt. % SNRI, up to 85 wt. % SNRI, up to 90 wt. % SNRI, up to 92 wt. % SNRI, up to 94 wt. % SNRI, up to 96 wt. % SNRI, up to 98 wt. % SNRI, up to 99 wt. % SNRI, or up to 99.5 wt. % SNRI.

In some embodiments, the unit dose can comprise a CGRP receptor antagonist at up to about 300 mg, e.g., up to 295 mg, up to 290 mg, up to 285 mg, up to 280 mg, up to 275 mg, up to 270 mg, up to 265 mg, up to 260 mg, up to 255 mg, up to 250 mg, up to 245 mg, up to 240 mg, up to 235 mg, up to 230 mg, up to 225 mg, up to 220 mg, up to 215 mg, up to 210 mg, up to 205 mg, up to 200 mg, up to 195 mg, up to 190 mg, up to 185 mg, up to 180 mg, up to 175 mg, up to 170 mg, up to 165 mg, up to 160 mg, up to 155 mg, up to 150 mg, up to 145 mg, up to 140 mg, up to 135 mg, up to 130 mg, up to 125 mg, up to 120 mg, up to 115 mg, up to 110 mg, up to 105 mg, up to 100 mg, up to 95 mg, up to 90 mg, up to 85 mg, up to 80 mg, up to 75 mg, up to 70 mg, up to 65 mg, up to 60 mg, up to 55 mg, up to 50 mg, up to 45 mg, up to 40 mg, up to 35 mg, up to 30 mg, up to 25 mg, up to 20 mg, up to 15 mg, up to 10 mg, up to 5 mg, up to 2 mg, up to 1 mg, up to 0.7 mg, up to 0.6 mg, up to 0.5 mg, up to 0.4 mg, up to 0.3 mg, up to 0.2 mg, or up to 0.1 mg. In terms of ranges, the unit dose can comprise a CGRP receptor antagonist from 0.1 mg to 300 mg, e.g., from 0.1 mg to 295 mg, from 0.5 mg to 295 mg, from 1 mg to 290 mg, from 5 mg to 290 mg, from 10 mg to 285 mg, from 20 mg to 285 mg, from 30 mg to 280 mg, from 40 mg to 270 mg, from 50 mg to 260 mg, from 60 mg to 250 mg, from 70 mg to 240 mg, from 80 mg to 230 mg, from 90 mg to 220 mg, from 100 mg to 210 mg, from 110 mg to 200 mg, from 120 mg to 190 mg, from 130 mg to 180 mg, from 140 mg to 170 mg, from 0.1 mg to 30 mg, from 0.1 mg to 40 mg, from 0.1 mg to 45 mg, from 0.1 mg to 50 mg, from 0.1 mg to 75 mg, from 0.1 mg to 100 mg, from 0.1 mg to 150 mg, from 100 mg to 150 mg, from 100 mg to 175 mg, from 100 mg to 200 mg, from 150 mg to 300 mg, from 150 mg to 275 mg, from 150 mg to 250 mg, from 150 mg to 225 mg, from 150 mg to 200 mg, from 200 mg to 300 mg, from 200 mg to 275 mg, or from 200 mg to 250 mg. In some embodiments, the unit dose comprises one or more additional active ingredients in addition to a CGRP receptor antagonist, and the total active ingredients in the unit dose comprise from 0.1 wt. % to 99.9 wt. % CGRP receptor antagonist, e.g., up to 0.5 wt. % CGRP receptor antagonist, up to 1 wt. % CGRP receptor antagonist, up to 2 wt. % CGRP receptor antagonist, up to 4 wt. % CGRP receptor antagonist, up to 6 wt. % CGRP receptor antagonist, up to 8 wt. % CGRP receptor antagonist, up to 10 wt. % CGRP receptor antagonist, up to 15 wt. % CGRP receptor antagonist, up to 20 wt. % CGRP receptor antagonist, up to 25 wt. % CGRP receptor antagonist, up to 30 wt. % CGRP receptor antagonist, up to 35 wt. % CGRP receptor antagonist, up to 40 wt. % CGRP receptor antagonist, up to 45 wt. % CGRP receptor antagonist, up to 50 wt. % CGRP receptor antagonist, up to 55 wt. % CGRP receptor antagonist, up to 60 wt. % CGRP receptor antagonist, up to 65 wt. % CGRP receptor antagonist, up to 70 wt. % CGRP receptor antagonist, up to 75 wt. % CGRP receptor antagonist, up to 80 wt. % CGRP receptor antagonist, up to 85 wt. % CGRP receptor antagonist, up to 90 wt. % CGRP receptor antagonist, up to 92 wt. % CGRP receptor antagonist, up to 94 wt. % CGRP receptor antagonist, up to 96 wt. % CGRP receptor antagonist, up to 98 wt. % CGRP receptor antagonist, up to 99 wt. % CGRP receptor antagonist, or up to 99.5 wt. % CGRP receptor antagonist.

In some embodiments, the unit dose can comprise an anti-CGRP monoclonal antibody at up to about 300 mg, e.g., up to 295 mg, up to 290 mg, up to 285 mg, up to 280 mg, up to 275 mg, up to 270 mg, up to 265 mg, up to 260 mg, up to 255 mg, up to 250 mg, up to 245 mg, up to 240 mg, up to 235 mg, up to 230 mg, up to 225 mg, up to 220 mg, up to 215 mg, up to 210 mg, up to 205 mg, up to 200 mg, up to 195 mg, up to 190 mg, up to 185 mg, up to 180 mg, up to 175 mg, up to 170 mg, up to 165 mg, up to 160 mg, up to 155 mg, up to 150 mg, up to 145 mg, up to 140 mg, up to 135 mg, up to 130 mg, up to 125 mg, up to 120 mg, up to 115 mg, up to 110 mg, up to 105 mg, up to 100 mg, up to 95 mg, up to 90 mg, up to 85 mg, up to 80 mg, up to 75 mg, up to 70 mg, up to 65 mg, up to 60 mg, up to 55 mg, up to 50 mg, up to 45 mg, up to 40 mg, up to 35 mg, up to 30 mg, up to 25 mg, up to 20 mg, up to 15 mg, up to 10 mg, up to 5 mg, up to 2 mg, up to 1 mg, up to 0.7 mg, up to 0.6 mg, up to 0.5 mg, up to 0.4 mg, up to 0.3 mg, up to 0.2 mg, or up to 0.1 mg. In terms of ranges, the unit dose can comprise an anti-CGRP monoclonal antibody from 0.1 mg to 300 mg, e.g., from 0.1 mg to 295 mg, from 0.5 mg to 295 mg, from 1 mg to 290 mg, from 5 mg to 290 mg, from 10 mg to 285 mg, from 20 mg to 285 mg, from 30 mg to 280 mg, from 40 mg to 270 mg, from 50 mg to 260 mg, from 60 mg to 250 mg, from 70 mg to 240 mg, from 80 mg to 230 mg, from 90 mg to 220 mg, from 100 mg to 210 mg, from 110 mg to 200 mg, from 120 mg to 190 mg, from 130 mg to 180 mg, from 140 mg to 170 mg, from 0.1 mg to 30 mg, from 0.1 mg to 40 mg, from 0.1 mg to 45 mg, from 0.1 mg to 50 mg, from 0.1 mg to 75 mg, from 0.1 mg to 100 mg, from 0.1 mg to 150 mg, from 100 mg to 150 mg, from 100 mg to 175 mg, from 100 mg to 200 mg, from 150 mg to 300 mg, from 150 mg to 275 mg, from 150 mg to 250 mg, from 150 mg to 225 mg, from 150 mg to 200 mg, from 200 mg to 300 mg, from 200 mg to 275 mg, or from 200 mg to 250 mg. In some embodiments, the unit dose comprises one or more additional active ingredients in addition to an anti-CGRP monoclonal antibody, and the total active ingredients in the unit dose comprise from 0.1 wt. % to 99.9 wt. % anti-CGRP monoclonal antibody, e.g., up to 0.5 wt. % anti-CGRP monoclonal antibody, up to 1 wt. % anti-CGRP monoclonal antibody, up to 2 wt. % anti-CGRP monoclonal antibody, up to 4 wt. % anti-CGRP monoclonal antibody, up to 6 wt. % anti-CGRP monoclonal antibody, up to 8 wt. % anti-CGRP monoclonal antibody, up to 10 wt. % anti-CGRP monoclonal antibody, up to 15 wt. % anti-CGRP monoclonal antibody, up to 20 wt. % anti-CGRP monoclonal antibody, up to 25 wt. % anti-CGRP monoclonal antibody, up to 30 wt. % anti-CGRP monoclonal antibody, up to 35 wt. % anti-CGRP monoclonal antibody, up to 40 wt. % anti-CGRP monoclonal antibody, up to 45 wt. % anti-CGRP monoclonal antibody, up to 50 wt. % anti-CGRP monoclonal antibody, up to 55 wt. % anti-CGRP monoclonal antibody, up to 60 wt. % anti-CGRP monoclonal antibody, up to 65 wt. % anti-CGRP monoclonal antibody, up to 70 wt. % anti-CGRP monoclonal antibody, up to 75 wt. % anti-CGRP monoclonal antibody, up to 80 wt. % anti-CGRP monoclonal antibody, up to 85 wt. % anti-CGRP monoclonal antibody, up to 90 wt. % anti-CGRP monoclonal antibody, up to 92 wt. % anti-CGRP monoclonal antibody, up to 94 wt. % anti-CGRP monoclonal antibody, up to 96 wt. % anti-CGRP monoclonal antibody, up to 98 wt. % anti-CGRP monoclonal antibody, up to 99 wt. % anti-CGRP monoclonal antibody, or up to 99.5 wt. % anti-CGRP monoclonal antibody.

In some embodiments, the unit dose can comprise an alpha-2C agonist at up to about 300 mg, e.g., up to 295 mg, up to 290 mg, up to 285 mg, up to 280 mg, up to 275 mg, up to 270 mg, up to 265 mg, up to 260 mg, up to 255 mg, up to 250 mg, up to 245 mg, up to 240 mg, up to 235 mg, up to 230 mg, up to 225 mg, up to 220 mg, up to 215 mg, up to 210 mg, up to 205 mg, up to 200 mg, up to 195 mg, up to 190 mg, up to 185 mg, up to 180 mg, up to 175 mg, up to 170 mg, up to 165 mg, up to 160 mg, up to 155 mg, up to 150 mg, up to 145 mg, up to 140 mg, up to 135 mg, up to 130 mg, up to 125 mg, up to 120 mg, up to 115 mg, up to 110 mg, up to 105 mg, up to 100 mg, up to 95 mg, up to 90 mg, up to 85 mg, up to 80 mg, up to 75 mg, up to 70 mg, up to 65 mg, up to 60 mg, up to 55 mg, up to 50 mg, up to 45 mg, up to 40 mg, up to 35 mg, up to 30 mg, up to 25 mg, up to 20 mg, up to 15 mg, up to 10 mg, up to 5 mg, up to 2 mg, up to 1 mg, up to 0.7 mg, up to 0.6 mg, up to 0.5 mg, up to 0.4 mg, up to 0.3 mg, up to 0.2 mg, or up to 0.1 mg. In terms of ranges, the unit dose can comprise an alpha-2C agonist from 0.1 mg to 300 mg, e.g., from 0.1 mg to 295 mg, from 0.5 mg to 295 mg, from 1 mg to 290 mg, from 5 mg to 290 mg, from 10 mg to 285 mg, from 20 mg to 285 mg, from 30 mg to 280 mg, from 40 mg to 270 mg, from 50 mg to 260 mg, from 60 mg to 250 mg, from 70 mg to 240 mg, from 80 mg to 230 mg, from 90 mg to 220 mg, from 100 mg to 210 mg, from 110 mg to 200 mg, from 120 mg to 190 mg, from 130 mg to 180 mg, from 140 mg to 170 mg, from 0.1 mg to 30 mg, from 0.1 mg to 40 mg, from 0.1 mg to 45 mg, from 0.1 mg to 50 mg, from 0.1 mg to 75 mg, from 0.1 mg to 100 mg, from 0.1 mg to 150 mg, from 100 mg to 150 mg, from 100 mg to 175 mg, from 100 mg to 200 mg, from 150 mg to 300 mg, from 150 mg to 275 mg, from 150 mg to 250 mg, from 150 mg to 225 mg, from 150 mg to 200 mg, from 200 mg to 300 mg, from 200 mg to 275 mg, or from 200 mg to 250 mg. In some embodiments, the unit dose comprises one or more additional active ingredients in addition to an alpha-2C agonist, and the total active ingredients in the unit dose comprise from 0.1 wt. % to 99.9 wt. % alpha-2C agonist, e.g., up to 0.5 wt. % alpha-2C agonist, up to 1 wt. % alpha-2C agonist, up to 2 wt. % alpha-2C agonist, up to 4 wt. % alpha-2C agonist, up to 6 wt. % alpha-2C agonist, up to 8 wt. % alpha-2C agonist, up to 10 wt. % alpha-2C agonist, up to 15 wt. % alpha-2C agonist, up to 20 wt. % alpha-2C agonist, up to 25 wt. % alpha-2C agonist, up to 30 wt. % alpha-2C agonist, up to 35 wt. % alpha-2C agonist, up to 40 wt. % alpha-2C agonist, up to 45 wt. % alpha-2C agonist, up to 50 wt. % alpha-2C agonist, up to 55 wt. % alpha-2C agonist, up to 60 wt. % alpha-2C agonist, up to 65 wt. % alpha-2C agonist, up to 70 wt. % alpha-2C agonist, up to 75 wt. % alpha-2C agonist, up to 80 wt. % alpha-2C agonist, up to 85 wt. % alpha-2C agonist, up to 90 wt. % alpha-2C agonist, up to 92 wt. % alpha-2C agonist, up to 94 wt. % alpha-2C agonist, up to 96 wt. % alpha-2C agonist, up to 98 wt. % alpha-2C agonist, up to 99 wt. % alpha-2C agonist, or up to 99.5 wt. % alpha-2C agonist.

In some embodiments, a unit dose disclosed herein comprises a combination of at least two components selected from the categories consisting of: 1) a serotonin-norepinephrine reuptake inhibitor (SNRI); 2) a calcitonin gene-related peptide (CGRP) receptor antagonist; 3) an anti-CGRP monoclonal antibody; and 4) an alpha-2C agonist. The at least two components may be present at any ratio relative to each other.

In some embodiments, the unit dose comprises an SNRI and a CGRP receptor antagonist, wherein the ratio of SNRI wt. % to CGRP receptor antagonist wt. % can be from about 10:1 to about 1:10, e.g., about 10:1 to about 1.5:1; about 10:1 to about 2:1; about 9:1 to about 1.5:1; about 9:1 to about 2:1; about 8:1 to about 1.5:1; about 8:1 to about 2:1; about 7:1 to about 2:1; about 5:1 to about 2:1; about 4:1 to about 2:1; or about 3:1 to about 2:1; about 1:2 to about 1:3; about 1:2 to about 1:4; about 1:2 to about 1:5; about 1:2 to about 1:7; about 1:8 to about 1:2; about 1:1.5 to about 1:8; about 1:2 to about 1:9; about 1:1.5 to about 1:9; about 1:2 to about 1:10; or about 1:1.5 to about 1:10. In some embodiments, the unit dose comprises an SNRI and an anti-CGRP monoclonal antibody, wherein the ratio of SNRI wt. % to anti-CGRP monoclonal antibody wt. % can be from about 10:1 to about 1:10, e.g., about 10:1 to about 1.5:1; about 10:1 to about 2:1; about 9:1 to about 1.5:1; about 9:1 to about 2:1; about 8:1 to about 1.5:1; about 8:1 to about 2:1; about 7:1 to about 2:1; about 5:1 to about 2:1; about 4:1 to about 2:1; or about 3:1 to about 2:1; about 1:2 to about 1:3; about 1:2 to about 1:4; about 1:2 to about 1:5; about 1:2 to about 1:7; about 1:8 to about 1:2; about 1:1.5 to about 1:8; about 1:2 to about 1:9; about 1:1.5 to about 1:9; about 1:2 to about 1:10; or about 1:1.5 to about 1:10. In some embodiments, the unit dose comprises an SNRI and an alpha-2C agonist, wherein the ratio of SNRI wt. % to alpha-2C agonist wt. % can be from about 10:1 to about 1:10, e.g., about 10:1 to about 1.5:1; about 10:1 to about 2:1; about 9:1 to about 1.5:1; about 9:1 to about 2:1; about 8:1 to about 1.5:1; about 8:1 to about 2:1; about 7:1 to about 2:1; about 5:1 to about 2:1; about 4:1 to about 2:1; or about 3:1 to about 2:1; about 1:2 to about 1:3; about 1:2 to about 1:4; about 1:2 to about 1:5; about 1:2 to about 1:7; about 1:8 to about 1:2; about 1:1.5 to about 1:8; about 1:2 to about 1:9; about 1:1.5 to about 1:9; about 1:2 to about 1:10; or about 1:1.5 to about 1:10.

In some embodiments, the unit dose comprises a CGRP receptor antagonist and an anti-CGRP monoclonal antibody, wherein the ratio of CGRP receptor antagonist wt. % to anti-CGRP monoclonal antibody wt. % can be from about 10:1 to about 1:10, e.g., about 10:1 to about 1.5:1; about 10:1 to about 2:1; about 9:1 to about 1.5:1; about 9:1 to about 2:1; about 8:1 to about 1.5:1; about 8:1 to about 2:1; about 7:1 to about 2:1; about 5:1 to about 2:1; about 4:1 to about 2:1; or about 3:1 to about 2:1; about 1:2 to about 1:3; about 1:2 to about 1:4; about 1:2 to about 1:5; about 1:2 to about 1:7; about 1:8 to about 1:2; about 1:1.5 to about 1:8; about 1:2 to about 1:9; about 1:1.5 to about 1:9; about 1:2 to about 1:10; or about 1:1.5 to about 1:10. In some embodiments, the unit dose comprises a CGRP receptor antagonist and an alpha-2C agonist, wherein the ratio of CGRP receptor antagonist wt. % to alpha-2C agonist wt. % can be from about 10:1 to about 1:10, e.g., about 10:1 to about 1.5:1; about 10:1 to about 2:1; about 9:1 to about 1.5:1; about 9:1 to about 2:1; about 8:1 to about 1.5:1; about 8:1 to about 2:1; about 7:1 to about 2:1; about 5:1 to about 2:1; about 4:1 to about 2:1; or about 3:1 to about 2:1; about 1:2 to about 1:3; about 1:2 to about 1:4; about 1:2 to about 1:5; about 1:2 to about 1:7; about 1:8 to about 1:2; about 1:1.5 to about 1:8; about 1:2 to about 1:9; about 1:1.5 to about 1:9; about 1:2 to about 1:10; or about 1:1.5 to about 1:10. In some embodiments, the unit dose comprises an anti-CGRP monoclonal antibody and an alpha-2C agonist, wherein the ratio of anti-CGRP monoclonal antibody wt. % to alpha-2C agonist wt. % can be from about 10:1 to about 1:10, e.g., about 10:1 to about 1.5:1; about 10:1 to about 2:1; about 9:1 to about 1.5:1; about 9:1 to about 2:1; about 8:1 to about 1.5:1; about 8:1 to about 2:1; about 7:1 to about 2:1; about 5:1 to about 2:1; about 4:1 to about 2:1; or about 3:1 to about 2:1; about 1:2 to about 1:3; about 1:2 to about 1:4; about 1:2 to about 1:5; about 1:2 to about 1:7; about 1:8 to about 1:2; about 1:1.5 to about 1:8; about 1:2 to about 1:9; about 1:1.5 to about 1:9; about 1:2 to about 1:10; or about 1:1.5 to about 1:10.

The active ingredient combinations disclosed herein are ideally suited for the preparation of a kit. In some embodiments, kits are provided for carrying out any of the methods described herein. The kits of this disclosure may comprise a carrier container being compartmentalized to receive in close confinement one or more containers such as vials, tubes, syringes, and the like, each of the containers comprising one of the separate elements to be used in the method.

An active ingredient combination as described in this disclosure for use in treating a subject may be delivered in a pharmaceutical package or kit to doctors and subjects. Such packaging is intended to improve patient convenience and compliance with the treatment plan. Typically the packaging comprises paper (cardboard) or plastic. In some embodiments, the kit or pharmaceutical package further comprises instructions for use (e.g., for administering according to a method as described herein).

In one embodiment, the kit or pharmaceutical package comprises the active ingredient combination in a defined, therapeutically effective dose in a single unit dosage form or as separate unit doses. The dose and form of the unit dose (e.g., tablet, capsule, immediate release, delayed release, etc.) can be any doses or forms as described herein. In some embodiments, the unit dose comprises two or more separate unit dosage forms, each comprising one or more of the active ingredients in the active ingredient combination. In some embodiments, the two or more separate unit dosage forms may be formulated for administration via the same or different routes (e.g., two unit dosage forms may be formulated for oral administration, or one may be formulated for oral administration while another is formulated for injection).

In one embodiment, the kit or pharmaceutical package includes doses suitable for multiple days of administration, such as one week, one month, or three months.

In certain embodiments, kits are provided for producing a single-dose administration unit. In certain embodiments, kits containing single or multi-chambered pre-filled syringes are included. In certain embodiments, kits containing one or more containers of a formulation described in this disclosure are included.

### III. Methods of Treating

In another aspect, provided herein is a method of treating a subject, comprising administering to the subject a therapeutically effective amount of the pharmaceutical compositions described above. In some embodiments, the subject has been diagnosed with a chronic pain disorder, e.g., FM, PTSD, ME/CFS, IBS, post-viral syndroms (e.g., long COVID), post-war syndromes (e.g., Post-Gulf War syndrome), environmentally implicated syndromes (e.g., chemical sensitivity syndrome and sick-building syndrome), or combinations thereof. In some embodiments, the subject has at least one MUS including but not limited to widespread chronic pain, fatigue, non-restorative sleep, cognitive dysfunction, or a combination thereof. In some embodiments, the composition is administered to the subject orally (e.g., sublingually), intravenously, or intramuscularly. As used herein, the term “subject” means a mammalian subject. Exemplary subjects include, but are not limited to humans, monkeys, dogs, cats, mice, rats, cows, pigs, birds, horses, camels, goats, and sheep.

As used herein, “treating” or “treatment” of any disease or disorder refers to preventing or ameliorating a disease or disorder in a subject or a symptom thereof. The term ameliorating refers to any therapeutically beneficial result in the treatment of a disease state, e.g., FM, lessening in the severity or progression, or curing thereof. Thus, treating or treatment includes ameliorating at least one physical parameter or symptom. Treating or treatment includes modulating the disease or disorder, either physically (e.g., stabilization of a discernible symptom) or physiologically (e.g., stabilization of a physical parameter) or both. Treating or treatment includes delaying, preventing increases in, or decreasing at least one symptom. Thus, in the disclosed methods, treatment can refer to a 10%, 20%, 30%, 40%, 50%, 60%, 70%, 80%, 90%, or 100% reduction in the severity of an established disease or condition or symptom of the disease or condition. For example, a method for treating fibromyalgia in a subject by administering a pharmaceutical composition as described in this disclosure is considered to be a treatment if there is a 10% reduction in one or more symptoms of fibromyalgia in a subject as compared to a control. Thus the reduction can be a 10%, 20%, 30%, 40%, 50%, 60%, 70%, 80%, 90%, 100%, or any percent reduction in between 10% and 100% as compared to native or control levels. In some embodiments, the pharmaceutical composition is administered to the subject until the subject exhibits amelioration of at least one symptom of FM (or any of the other listed disorders). It is understood that treatment does not necessarily refer to a cure or complete ablation of the disease, condition, or symptoms of the disease or condition.

“Administering” or “administration of” a composition to a subject (and grammatical equivalents of this phrase), as used herein, refer to the act of injecting or otherwise physically delivering a substance as it exists outside the body (e.g., a pharmaceutical composition comprising a combination of active ingredients and any additional components, as described above) into a subject. Administration can be via enteral or parenteral routes. In some embodiments, administration is by mucosal, intradermal, intravenous, intramuscular, subcutaneous delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, delayed, or reduced in severity, administration of the substance typically occurs before the onset of the disease or symptoms thereof. Administration refers to direct administration, which may be administration to a subject by a medical professional or may be self-administration, and/or indirect administration, which may be the act of prescribing a composition. For example, a physician who instructs a subject to self-administer a composition and/or provides a subject with a prescription for a composition is administering the composition to the subject.

The compositions can be administered to a subject, e.g., a human subject, using a variety of methods that depend, in part, on the route of administration. The route can be, e.g., intravenous injection or infusion (IV), subcutaneous injection (SC), intraperitoneal (IP) injection, intramuscular injection (IM), intradermal injection (ID), subcutaneous, transdermal, intracavity, oral, intracranial injection, intrathecal injection (IT), or by topical delivery. The injection can be in a bolus or a continuous infusion. Techniques for preparing injectate or infusate delivery systems containing the active ingredients described herein are well known to those of skill in the art. Generally, such systems should utilize components that will not significantly impair the biological properties of the active ingredients (see, for example, Remington's Pharmaceutical Sciences, 18th edition, 1990, Mack Publishing). Those of skill in the art can readily determine the various parameters and conditions for producing injectates or infusates without resorting to undue experimentation. Administration can be achieved by, e.g., topical administration, local administration, injection, by means of an implant.

As used herein, the term “therapeutically effective amount” refers to an amount of pharmaceutical composition as described herein that, when administered to a subject, is effective to achieve an intended purpose, e.g., to reduce or ameliorate at least one symptom of a chronic pain disorder, prevent worsening of at least one symptom of a chronic pain disorder, and/or otherwise reduce the length of time that a patient experiences a symptom of a chronic pain disorder, or extend the length of time before a symptom may recur. A therapeutically effective amount is not, however, a dosage so large as to cause adverse side effects, such as hyperviscosity syndromes, pulmonary edema, congestive heart failure, and the like. A therapeutically effective amount may vary with the subject's age, condition, and sex, as well as the extent of the disease in the subject and can be determined by one of skill in the art. Other factors can include, e.g., other medical disorders concurrently or previously affecting the subject, the general health of the subject, the genetic disposition of the subject, diet, time of administration, rate of excretion, drug combination, and any other additional therapeutics or treatments that are administered to the subject. Although individual needs may vary, determination of optimal ranges for effective amounts of formulations is within the skill of the art. Human doses can be extrapolated from animal studies. Generally, the dosage required to provide an effective amount of a formulation, which can be adjusted by one skilled in the art, will vary depending on the age, health, physical condition, weight, type and extent of the disease or disorder of the recipient, frequency of treatment, the nature of concurrent therapy (if any), the method of administration, and the nature and scope of the desired effect(s) (Nies et ah, Chapter 3 In: Goodman & Gilman's The Pharmacological Basis of Therapeutics, 9th Ed., Hardman et ah, eds., McGraw-Hill, New York, NY, 1996). It should also be understood that a specific dosage and treatment regimen for any particular subject also depends upon the judgment of the treating medical practitioner (e.g., doctor or nurse). A therapeutically effective amount is also one in which any toxic or detrimental effects of the composition are outweighed by the therapeutically beneficial effects. The dosage of the therapeutically effective amount may be adjusted by the individual physician or veterinarian in the event of any complication. In some instances, a therapeutically effective amount may vary from about 0.01 mg to about 500 mg, e.g. from 1 mg to 400 mg, from 5 mg to 350 mg, from 10 mg to 300 mg, from 0.01 mg to 100 mg, from 0.01 mg to 200 mg, from 0.01 mg to 250 mg, from 100 mg to 400 mg, from 150 mg to 300 mg, from 200 mg to 500 mg, from 250 mg to 500 mg, or from 300 mg to 500 mg, in one or more dose administrations daily, for one or several days.

In some embodiments, a therapeutically effective amount of the pharmaceutical compositions comprises at least one active ingredient component at a lower dose than the standard dose for said active ingredient. In some embodiments, the therapeutically effective amount comprises an active ingredient at less than 100% of the standard dose, e.g., less than 99% of the standard dose, less than 98% of the standard dose, less than 96% of the standard dose, less than 94% of the standard dose, less than 92% of the standard dose, less than 90% of the standard dose, less than 85% of the standard dose, less than 80% of the standard dose, less than 75% of the standard dose, less than 70% of the standard dose, less than 65% of the standard dose, less than 60% of the standard dose, less than 55% of the standard dose, less than 50% of the standard dose, less than 45% of the standard dose, less than 40% of the standard dose, less than 35% of the standard dose, less than 30% of the standard dose, less than 25% of the standard dose, less than 20% of the standard dose, less than 15% of the standard dose, less than 10% of the standard dose, or less than 5% of the standard dose. In some embodiments, administration of the active ingredient at lower doses can prevent undesirable side effects.

In some embodiments, the pharmaceutical composition is administered to the subject 1 time/day. In some embodiments, the pharmaceutical composition is administered to the subject multiple times/day, e.g., 2 times/day, 3 times/day, or 4 times/day. In some embodiments, the pharmaceutical composition is administered to the subject from 1 time/year to 1 time/day, e.g., 1 time/year, 2 times/year, 4 times/year, 6 times/year, 12 times/year, 1 time/six months, 1 time/four months, 1 time/3 months, 1 time/2 months, 1 time/month, 2 times/month, 4 times/month, 8 times/month, 1 time/4 weeks, 1 time/3 weeks, 1 time/2 weeks, 1 time/week, 2 times/week, 3 times/week, 4 times/week, 5 times/week, 6 times/week, 7 times/week, or 1 time/day. In some embodiments, the pharmaceutical composition is administered on consecutive days or on non-consecutive days. In some embodiments, the pharmaceutical composition is administered to the subject for at least 1 day, at least 2 days, at least 4 days, at least 5 days, at least 6 days, at least 1 week, at least 2 weeks, at least 3 weeks, at least 1 month, at least 2 months, or at least 3 months.

A pharmaceutical preparation as described herein can comprise a therapeutically effective amount of at least two of the active ingredients in combination as described herein. Such effective amounts can be determined by one of ordinary skill in the art as described above. Considerations include the effect of the administered pharmaceutical composition, or the combinatorial effect of the active ingredients therein.

In some embodiments, a therapeutically effective amount of a pharmaceutical composition described herein can comprise up to about 120 mg duloxetine or up to about 60 mg duloxetine, e.g., up to 58 mg duloxetine, up to 56 mg duloxetine, up to 54 mg duloxetine, up to 52 mg duloxetine, up to 50 mg duloxetine, up to 48 mg duloxetine, up to 46 mg duloxetine, up to 44 mg duloxetine, up to 42 mg duloxetine, up to 40 mg duloxetine, up to 38 mg duloxetine, up to 36 mg duloxetine, up to 34 mg duloxetine, up to 32 mg duloxetine, up to 30 mg duloxetine, up to 28 mg duloxetine, up to 26 mg duloxetine, up to 24 mg duloxetine, up to 22 mg duloxetine, up to 20 mg duloxetine, up to 18 mg duloxetine, up to 16 mg duloxetine, up to 14 mg duloxetine, up to 12 mg duloxetine, or up to 10 mg duloxetine. In terms of ranges, the therapeutically effective amount can comprise from 10 mg to 60 mg duloxetine, e.g., from 15 mg to 55 mg duloxetine, from 20 mg to 50 mg duloxetine, from 25 mg to 45 mg duloxetine, from 30 mg to 40 mg duloxetine, from 10 mg to 30 mg duloxetine, from 10 mg to 20 mg duloxetine, from 30 mg to 45 mg duloxetine, from 30 mg to 50 mg duloxetine, from 30 mg to 60 mg duloxetine, from 40 mg to 60 mg duloxetine, or from 50 mg to 60 mg duloxetine. In some embodiments, the therapeutically effective amount comprises one or more additional active ingredients in addition to duloxetine and the total active ingredients in the therapeutically effective amount comprise from 0.1 wt. % to 99.9 wt. % duloxetine, e.g., up to 0.5 wt. % duloxetine, up to 1 wt. % duloxetine, up to 2 wt. % duloxetine, up to 4 wt. % duloxetine, up to 6 wt. % duloxetine, up to 8 wt. % duloxetine, up to 10 wt. % duloxetine, up to 15 wt. % duloxetine, up to 20 wt. % duloxetine, up to 25 wt. % duloxetine, up to 30 wt. % duloxetine, up to 35 wt. % duloxetine, up to 40 wt. % duloxetine, up to 45 wt. % duloxetine, up to 50 wt. % duloxetine, up to 55 wt. % duloxetine, up to 60 wt. % duloxetine, up to 65 wt. % duloxetine, up to 70 wt. % duloxetine, up to 75 wt. % duloxetine, up to 80 wt. % duloxetine, up to 85 wt. % duloxetine, up to 90 wt. % duloxetine, up to 92 wt. % duloxetine, up to 94 wt. % duloxetine, up to 96 wt. % duloxetine, up to 98 wt. % duloxetine, up to 99 wt. % duloxetine, or up to 99.5 wt. % duloxetine. In some embodiments, the pharmaceutical composition comprising duloxetine is administered to the subject 1 time/day.

In some embodiments, a therapeutically effective amount of a pharmaceutical composition described herein can comprise up to 190 mg milnacipran, up to 185 mg milnacipran, up to 180 mg milnacipran, up to 175 mg milnacipran, up to 170 mg milnacipran, up to 165 mg milnacipran, up to 160 mg milnacipran, up to 155 mg milnacipran, up to 150 mg milnacipran, up to 145 mg milnacipran, up to 140 mg milnacipran, up to 135 mg milnacipran, up to 130 mg milnacipran, up to 125 mg milnacipran, up to 120 mg milnacipran, up to 115 mg milnacipran, up to 110 mg milnacipran, up to 105 mg milnacipran, up to 100 mg milnacipran, up to 95 mg milnacipran, up to 90 mg milnacipran, up to 85 mg milnacipran, up to 80 mg milnacipran, up to 75 mg milnacipran, up to 70 mg milnacipran, up to 65 mg milnacipran, up to 60 mg milnacipran, up to 55 mg milnacipran, up to 50 mg milnacipran, up to 45 mg milnacipran, up to 40 mg milnacipran, up to 35 mg milnacipran, up to 30 mg milnacipran, up to 25 mg milnacipran, up to 20 mg milnacipran, up to 15 mg milnacipran, or up to 10 mg milnacipran. In terms of ranges, the therapeutically effective amount can comprise from 10 mg to 200 mg milnacipran, e.g., from 20 mg to 190 mg milnacipran, from 30 mg to 180 mg milnacipran, from 40 mg to 170 mg milnacipran, from 50 mg to 160 mg milnacipran, from 60 mg to 150 mg milnacipran, from 70 mg to 140 mg milnacipran, from 80 mg to 130 mg milnacipran, from 90 mg to 120 mg milnacipran, from 100 mg to 110 mg milnacipran, from 10 mg to 100 mg milnacipran, from 15 mg to 90 mg milnacipran, from 20 mg to 80 mg milnacipran, from 25 mg to 75 mg milnacipran, from 100 mg to 200 mg milnacipran, from 110 mg to 190 mg milnacipran, from 120 mg to 180 mg milnacipran, from 125 mg to 175 mg milnacipran, from 130 mg to 165 mg milnacipran, from 50 mg to 150 mg milnacipran, from 60 mg to 150 mg milnacipran, from 75 mg to 150 mg milnacipran, or from 75 mg to 125 mg milnacipran. In some embodiments, the therapeutically effective amount comprises one or more additional active ingredients in addition to milnacipran and the total active ingredients in the therapeutically effective amount comprise from 0.1 wt. % to 99.9 wt. % milnacipran, e.g., up to 0.5 wt. % milnacipran, up to 1 wt. % milnacipran, up to 2 wt. % milnacipran, up to 4 wt. % milnacipran, up to 6 wt. % milnacipran, up to 8 wt. % milnacipran, up to 10 wt. % milnacipran, up to 15 wt. % milnacipran, up to 20 wt. % milnacipran, up to 25 wt. % milnacipran, up to 30 wt. % milnacipran, up to 35 wt. % milnacipran, up to 40 wt. % milnacipran, up to 45 wt. % milnacipran, up to 50 wt. % milnacipran, up to 55 wt. % milnacipran, up to 60 wt. % milnacipran, up to 65 wt. % milnacipran, up to 70 wt. % milnacipran, up to 75 wt. % milnacipran, up to 80 wt. % milnacipran, up to 85 wt. % milnacipran, up to 90 wt. % milnacipran, up to 92 wt. % milnacipran, up to 94 wt. % milnacipran, up to 96 wt. % milnacipran, up to 98 wt. % milnacipran, up to 99 wt. % milnacipran, or up to 99.5 wt. % milnacipran. In some embodiments, the pharmaceutical composition comprising milnacipran is administered to the subject 1 time/day.

In some embodiments, a therapeutically effective amount of a pharmaceutical composition described herein can comprise up to about 75 mg rimegepant, e.g., up to 74 mg rimegepant, up to 73 mg rimegepant, up to 72 mg rimegepant, up to 70 mg rimegepant, up to 68 mg rimegepant, up to 66 mg rimegepant, up to 64 mg rimegepant, up to 62 mg rimegepant, up to 60 mg rimegepant, up to 58 mg rimegepant, up to 56 mg rimegepant, up to 54 mg rimegepant, up to 52 mg rimegepant, up to 50 mg rimegepant, up to 48 mg rimegepant, up to 46 mg rimegepant, up to 44 mg rimegepant, up to 42 mg rimegepant, up to 40 mg rimegepant, up to 38 mg rimegepant, up to 36 mg rimegepant, up to 34 mg rimegepant, up to 32 mg rimegepant, up to 30 mg rimegepant, up to 28 mg rimegepant, up to 26 mg rimegepant, up to 24 mg rimegepant, up to 22 mg rimegepant, up to 20 mg rimegepant, up to 18 mg rimegepant, up to 16 mg rimegepant, up to 14 mg rimegepant, up to 12 mg rimegepant, or up to 10 mg rimegepant. In terms of ranges, the therapeutically effective amount can comprise from 10 mg to 75 mg rimegepant, e.g., from 15 mg to 70 mg rimegepant, from 20 mg to 65 mg rimegepant, from 25 mg to 60 mg rimegepant, from 30 mg to 55 mg rimegepant, from 10 mg to 40 mg rimegepant, from 10 mg to 30 mg rimegepant, from 10 mg to 20 mg rimegepant, from 30 mg to 45 mg rimegepant, from 30 mg to 50 mg rimegepant, from 30 mg to 60 mg rimegepant, from 30 mg to 70 mg rimegepant, from 30 mg to 75 mg rimegepant, from 40 mg to 75 mg rimegepant, from 50 mg to 75 mg rimegepant, or from 60 mg to 75 mg rimegepant. In some embodiments, the therapeutically effective amount comprises one or more additional active ingredients in addition to rimegepant and the total active ingredients in the therapeutically effective amount comprise from 0.1 wt. % to 99.9 wt. % rimegepant, e.g., up to 0.5 wt. % rimegepant, up to 1 wt. % rimegepant, up to 2 wt. % rimegepant, up to 4 wt. % rimegepant, up to 6 wt. % rimegepant, up to 8 wt. % rimegepant, up to 10 wt. % rimegepant, up to 15 wt. % rimegepant, up to 20 wt. % rimegepant, up to 25 wt. % rimegepant, up to 30 wt. % rimegepant, up to 35 wt. % rimegepant, up to 40 wt. % rimegepant, up to 45 wt. % rimegepant, up to 50 wt. % rimegepant, up to 55 wt. % rimegepant, up to 60 wt. % rimegepant, up to 65 wt. % rimegepant, up to 70 wt. % rimegepant, up to 75 wt. % rimegepant, up to 80 wt. % rimegepant, up to 85 wt. % rimegepant, up to 90 wt. % rimegepant, up to 92 wt. % rimegepant, up to 94 wt. % rimegepant, up to 96 wt. % rimegepant, up to 98 wt. % rimegepant, up to 99 wt. % rimegepant, or up to 99.5 wt. % rimegepant. In some embodiments, the pharmaceutical composition comprising rimegepant is administered to the subject 1 time/day.

In some embodiments, a therapeutically effective amount of a pharmaceutical composition described herein can comprise up to about 200 mg ubrogepant, e.g., up to 195 mg ubrogepant, up to 190 mg ubrogepant, up to 185 mg ubrogepant, up to 180 mg ubrogepant, up to 175 mg ubrogepant, up to 170 mg ubrogepant, up to 165 mg ubrogepant, up to 160 mg ubrogepant, up to 155 mg ubrogepant, up to 150 mg ubrogepant, up to 145 mg ubrogepant, up to 140 mg ubrogepant, up to 135 mg ubrogepant, up to 130 mg ubrogepant, up to 125 mg ubrogepant, up to 120 mg ubrogepant, up to 115 mg ubrogepant, up to 110 mg ubrogepant, up to 105 mg ubrogepant, up to 100 mg ubrogepant, up to 95 mg ubrogepant, up to 90 mg ubrogepant, up to 85 mg ubrogepant, up to 80 mg ubrogepant, up to 75 mg ubrogepant, up to 70 mg ubrogepant, up to 65 mg ubrogepant, up to 60 mg ubrogepant, up to 55 mg ubrogepant, up to 50 mg ubrogepant, up to 45 mg ubrogepant, up to 40 mg ubrogepant, up to 35 mg ubrogepant, up to 30 mg ubrogepant, up to 25 mg ubrogepant, up to 20 mg ubrogepant, up to 15 mg ubrogepant, or up to 10 mg ubrogepant. In terms of ranges, the therapeutically effective amount can comprise from 10 mg to 200 mg ubrogepant, e.g., from 20 mg to 190 mg ubrogepant, from 30 mg to 180 mg ubrogepant, from 40 mg to 170 mg ubrogepant, from 50 mg to 160 mg ubrogepant, from 60 mg to 150 mg ubrogepant, from 70 mg to 140 mg ubrogepant, from 80 mg to 130 mg ubrogepant, from 90 mg to 120 mg ubrogepant, from 100 mg to 110 mg ubrogepant, from 10 mg to 30 mg ubrogepant, from 10 mg to 40 mg ubrogepant, from 10 mg to 45 mg ubrogepant, from 10 mg to 50 mg ubrogepant, from 10 mg to 75 mg ubrogepant, from 10 mg to 100 mg ubrogepant, from 15 mg to 90 mg ubrogepant, from 20 mg to 80 mg ubrogepant, from 25 mg to 75 mg ubrogepant, from 100 mg to 200 mg ubrogepant, from 110 mg to 190 mg ubrogepant, from 120 mg to 180 mg ubrogepant, from 125 mg to 175 mg ubrogepant, from 130 mg to 165 mg ubrogepant, from 50 mg to 150 mg ubrogepant, from 60 mg to 150 mg ubrogepant, from 75 mg to 150 mg ubrogepant, or from 75 mg to 125 mg ubrogepant. In some embodiments, the therapeutically effective amount comprises one or more additional active ingredients in addition to ubrogepant and the total active ingredients in the therapeutically effective amount comprise from 0.1 wt. % to 99.9 wt. % ubrogepant, e.g., up to 0.5 wt. % ubrogepant, up to 1 wt. % ubrogepant, up to 2 wt. % ubrogepant, up to 4 wt. % ubrogepant, up to 6 wt. % ubrogepant, up to 8 wt. % ubrogepant, up to 10 wt. % ubrogepant, up to 15 wt. % ubrogepant, up to 20 wt. % ubrogepant, up to 25 wt. % ubrogepant, up to 30 wt. % ubrogepant, up to 35 wt. % ubrogepant, up to 40 wt. % ubrogepant, up to 45 wt. % ubrogepant, up to 50 wt. % ubrogepant, up to 55 wt. % ubrogepant, up to 60 wt. % ubrogepant, up to 65 wt. % ubrogepant, up to 70 wt. % ubrogepant, up to 75 wt. % ubrogepant, up to 80 wt. % ubrogepant, up to 85 wt. % ubrogepant, up to 90 wt. % ubrogepant, up to 92 wt. % ubrogepant, up to 94 wt. % ubrogepant, up to 96 wt. % ubrogepant, up to 98 wt. % ubrogepant, up to 99 wt. % ubrogepant, or up to 99.5 wt. % ubrogepant. In some embodiments, the pharmaceutical composition comprising ubrogepant is administered to the subject 1 time/day.

In some embodiments, a therapeutically effective amount of a pharmaceutical composition described herein can comprise up to about 60 mg atogepant, e.g., up to 58 mg atogepant, up to 56 mg atogepant, up to 54 mg atogepant, up to 52 mg atogepant, up to 50 mg atogepant, up to 48 mg atogepant, up to 46 mg atogepant, up to 44 mg atogepant, up to 42 mg atogepant, up to 40 mg atogepant, up to 38 mg atogepant, up to 36 mg atogepant, up to 34 mg atogepant, up to 32 mg atogepant, up to 30 mg atogepant, up to 28 mg atogepant, up to 26 mg atogepant, up to 24 mg atogepant, up to 22 mg atogepant, up to 20 mg atogepant, up to 18 mg atogepant, up to 16 mg atogepant, up to 14 mg atogepant, up to 12 mg atogepant, or up to 10 mg atogepant. In terms of ranges, the therapeutically effective amount can comprise from 10 mg to 60 mg atogepant, e.g., from 15 mg to 55 mg atogepant, from 20 mg to 50 mg atogepant, from 25 mg to 45 mg atogepant, from 30 mg to 40 mg atogepant, from 10 mg to 30 mg atogepant, from 10 mg to 20 mg atogepant, from 30 mg to 45 mg atogepant, from 30 mg to 50 mg atogepant, from 30 mg to 60 mg atogepant, from 40 mg to 60 mg atogepant, or from 50 mg to 60 mg atogepant. In some embodiments, the therapeutically effective amount comprises one or more additional active ingredients in addition to atogepant and the total active ingredients in the therapeutically effective amount comprise from 0.1 wt. % to 99.9 wt. % atogepant, e.g., up to 0.5 wt. % atogepant, up to 1 wt. % atogepant, up to 2 wt. % atogepant, up to 4 wt. % atogepant, up to 6 wt. % atogepant, up to 8 wt. % atogepant, up to 10 wt. % atogepant, up to 15 wt. % atogepant, up to 20 wt. % atogepant, up to 25 wt. % atogepant, up to 30 wt. % atogepant, up to 35 wt. % atogepant, up to 40 wt. % atogepant, up to 45 wt. % atogepant, up to 50 wt. % atogepant, up to 55 wt. % atogepant, up to 60 wt. % atogepant, up to 65 wt. % atogepant, up to 70 wt. % atogepant, up to 75 wt. % atogepant, up to 80 wt. % atogepant, up to 85 wt. % atogepant, up to 90 wt. % atogepant, up to 92 wt. % atogepant, up to 94 wt. % atogepant, up to 96 wt. % atogepant, up to 98 wt. % atogepant, up to 99 wt. % atogepant, or up to 99.5 wt. % atogepant. In some embodiments, the pharmaceutical composition comprising atogepant is administered to the subject 1 time/day.

In some embodiments, a therapeutically effective amount of a pharmaceutical composition described herein can comprise up to about 140 mg erenumab, e.g., up to 139 mg erenumab, up to 138 mg erenumab, up to 137 mg erenumab, up to 135 mg erenumab, up to 130 mg erenumab, up to 125 mg erenumab, up to 120 mg erenumab, up to 115 mg erenumab, up to 110 mg erenumab, up to 105 mg erenumab, up to 100 mg erenumab, up to 95 mg erenumab, up to 90 mg erenumab, up to 85 mg erenumab, up to 80 mg erenumab, up to 75 mg erenumab, up to 70 mg erenumab, up to 65 mg erenumab, up to 60 mg erenumab, up to 55 mg erenumab, up to 50 mg erenumab, up to 45 mg erenumab, up to 40 mg erenumab, up to 35 mg erenumab, up to 30 mg erenumab, up to 25 mg erenumab, up to 20 mg erenumab, up to 15 mg erenumab, or up to 10 mg erenumab. In terms of ranges, the therapeutically effective amount can comprise from 10 mg to 140 mg erenumab, e.g., from 15 mg to 135 mg erenumab, from 20 mg to 130 mg erenumab, from 25 mg to 125 mg erenumab, from 30 mg to 120 mg erenumab, from 35 mg to 115 mg erenumab, from 40 mg to 110 mg erenumab, from 45 mg to 105 mg erenumab, from 50 mg to 100 mg erenumab, from 55 mg to 95 mg erenumab, from 60 mg to 90 mg erenumab, from 10 mg to 30 mg erenumab, from 10 mg to 40 mg erenumab, from 10 mg to 45 mg erenumab, from 10 mg to 50 mg erenumab, from 10 mg to 60 mg erenumab, from 10 mg to 70 mg erenumab, from 10 mg to 75 mg erenumab, from 10 mg to 100 mg erenumab, from 15 mg to 90 mg erenumab, from 20 mg to 80 mg erenumab, from 25 mg to 75 mg erenumab, from 70 mg to 120 mg erenumab, from 70 mg to 125 mg erenumab, from 70 mg to 130 mg erenumab, from 70 mg to 135 mg erenumab, or from 70 mg to 140 mg erenumab. In some embodiments, the therapeutically effective amount comprises one or more additional active ingredients in addition to erenumab and the total active ingredients in the therapeutically effective amount comprise from 0.1 wt. % to 99.9 wt. % erenumab, e.g., up to 0.5 wt. % erenumab, up to 1 wt. % erenumab, up to 2 wt. % erenumab, up to 4 wt. % erenumab, up to 6 wt. % erenumab, up to 8 wt. % erenumab, up to 10 wt. % erenumab, up to 15 wt. % erenumab, up to 20 wt. % erenumab, up to 25 wt. % erenumab, up to 30 wt. % erenumab, up to 35 wt. % erenumab, up to 40 wt. % erenumab, up to 45 wt. % erenumab, up to 50 wt. % erenumab, up to 55 wt. % erenumab, up to 60 wt. % erenumab, up to 65 wt. % erenumab, up to 70 wt. % erenumab, up to 75 wt. % erenumab, up to 80 wt. % erenumab, up to 85 wt. % erenumab, up to 90 wt. % erenumab, up to 92 wt. % erenumab, up to 94 wt. % erenumab, up to 96 wt. % erenumab, up to 98 wt. % erenumab, up to 99 wt. % erenumab, or up to 99.5 wt. % erenumab. In some embodiments, the pharmaceutical composition comprising erenumab is administered to the subject 1 time/month.

In some embodiments, a therapeutically effective amount of a pharmaceutical composition described herein can comprise up to about 300 mg eptinezumab, e.g., up to 295 mg eptinezumab, up to 290 mg eptinezumab, up to 285 mg eptinezumab, up to 280 mg eptinezumab, up to 275 mg eptinezumab, up to 270 mg eptinezumab, up to 265 mg eptinezumab, up to 260 mg eptinezumab, up to 255 mg eptinezumab, up to 250 mg eptinezumab, up to 245 mg eptinezumab, up to 240 mg eptinezumab, up to 235 mg eptinezumab, up to 230 mg eptinezumab, up to 225 mg eptinezumab, up to 220 mg eptinezumab, up to 215 mg eptinezumab, up to 210 mg eptinezumab, up to 205 mg eptinezumab, up to 200 mg eptinezumab, up to 195 mg eptinezumab, up to 190 mg eptinezumab, up to 185 mg eptinezumab, up to 180 mg eptinezumab, up to 175 mg eptinezumab, up to 170 mg eptinezumab, up to 165 mg eptinezumab, up to 160 mg eptinezumab, up to 155 mg eptinezumab, up to 150 mg eptinezumab, up to 145 mg eptinezumab, up to 140 mg eptinezumab, up to 135 mg eptinezumab, up to 130 mg eptinezumab, up to 125 mg eptinezumab, up to 120 mg eptinezumab, up to 115 mg eptinezumab, up to 110 mg eptinezumab, up to 105 mg eptinezumab, up to 100 mg eptinezumab, up to 95 mg eptinezumab, up to 90 mg eptinezumab, up to 85 mg eptinezumab, up to 80 mg eptinezumab, up to 75 mg eptinezumab, up to 70 mg eptinezumab, up to 65 mg eptinezumab, up to 60 mg eptinezumab, up to 55 mg eptinezumab, up to 50 mg eptinezumab, up to 45 mg eptinezumab, up to 40 mg eptinezumab, up to 35 mg eptinezumab, up to 30 mg eptinezumab, up to 25 mg eptinezumab, up to 20 mg eptinezumab, up to 15 mg eptinezumab, or up to 10 mg eptinezumab. In terms of ranges, the therapeutically effective amount can comprise from 10 mg to 300 mg eptinezumab, e.g., from 20 mg to 290 mg eptinezumab, from 30 mg to 280 mg eptinezumab, from 40 mg to 270 mg eptinezumab, from 50 mg to 260 mg eptinezumab, from 60 mg to 250 mg eptinezumab, from 70 mg to 240 mg eptinezumab, from 80 mg to 230 mg eptinezumab, from 90 mg to 220 mg eptinezumab, from 100 mg to 210 mg eptinezumab, from 110 mg to 200 mg eptinezumab, from 120 mg to 190 mg eptinezumab, from 130 mg to 180 mg eptinezumab, from 140 mg to 170 mg eptinezumab, from 10 mg to 30 mg eptinezumab, from 10 mg to 40 mg eptinezumab, from 10 mg to 45 mg eptinezumab, from 10 mg to 50 mg eptinezumab, from 10 mg to 75 mg eptinezumab, from 10 mg to 100 mg eptinezumab, from 10 mg to 150 mg eptinezumab, from 100 mg to 150 mg eptinezumab, from 100 mg to 175 mg eptinezumab, from 100 mg to 200 mg eptinezumab, from 150 mg to 300 mg eptinezumab, from 150 mg to 275 mg eptinezumab, from 150 mg to 250 mg eptinezumab, from 150 mg to 225 mg eptinezumab, from 150 mg to 200 mg eptinezumab, from 200 mg to 300 mg eptinezumab, from 200 mg to 275 mg eptinezumab, or from 200 mg to 250 mg eptinezumab. In some embodiments, the therapeutically effective amount comprises one or more additional active ingredients in addition to eptinezumab and the total active ingredients in the therapeutically effective amount comprise from 0.1 wt. % to 99.9 wt. % eptinezumab, e.g., up to 0.5 wt. % eptinezumab, up to 1 wt. % eptinezumab, up to 2 wt. % eptinezumab, up to 4 wt. % eptinezumab, up to 6 wt. % eptinezumab, up to 8 wt. % eptinezumab, up to 10 wt. % eptinezumab, up to 15 wt. % eptinezumab, up to 20 wt. % eptinezumab, up to 25 wt. % eptinezumab, up to 30 wt. % eptinezumab, up to 35 wt. % eptinezumab, up to 40 wt. % eptinezumab, up to 45 wt. % eptinezumab, up to 50 wt. % eptinezumab, up to 55 wt. % eptinezumab, up to 60 wt. % eptinezumab, up to 65 wt. % eptinezumab, up to 70 wt. % eptinezumab, up to 75 wt. % eptinezumab, up to 80 wt. % eptinezumab, up to 85 wt. % eptinezumab, up to 90 wt. % eptinezumab, up to 92 wt. % eptinezumab, up to 94 wt. % eptinezumab, up to 96 wt. % eptinezumab, up to 98 wt. % eptinezumab, up to 99 wt. % eptinezumab, or up to 99.5 wt. % eptinezumab. In some embodiments, the pharmaceutical composition comprising eptinezumab is administered to the subject 1 time/month. In some embodiments, the pharmaceutical composition comprising eptinezumab is administered to the subject 1 time/3 months.

In some embodiments, a therapeutically effective amount of a pharmaceutical composition described herein can comprise up to about 225 mg fremanezumab, e.g., up to 220 mg fremanezumab, up to 215 mg fremanezumab, up to 210 mg fremanezumab, up to 205 mg fremanezumab, up to 200 mg fremanezumab, up to 195 mg fremanezumab, up to 190 mg fremanezumab, up to 185 mg fremanezumab, up to 180 mg fremanezumab, up to 175 mg fremanezumab, up to 170 mg fremanezumab, up to 165 mg fremanezumab, up to 160 mg fremanezumab, up to 155 mg fremanezumab, up to 150 mg fremanezumab, up to 145 mg fremanezumab, up to 140 mg fremanezumab, up to 135 mg fremanezumab, up to 130 mg fremanezumab, up to 125 mg fremanezumab, up to 120 mg fremanezumab, up to 115 mg fremanezumab, up to 110 mg fremanezumab, up to 105 mg fremanezumab, up to 100 mg fremanezumab, up to 95 mg fremanezumab, up to 90 mg fremanezumab, up to 85 mg fremanezumab, up to 80 mg fremanezumab, up to 75 mg fremanezumab, up to 70 mg fremanezumab, up to 65 mg fremanezumab, up to 60 mg fremanezumab, up to 55 mg fremanezumab, up to 50 mg fremanezumab, up to 45 mg fremanezumab, up to 40 mg fremanezumab, up to 35 mg fremanezumab, up to 30 mg fremanezumab, up to 25 mg fremanezumab, up to 20 mg fremanezumab, up to 15 mg fremanezumab, or up to 10 mg fremanezumab. In terms of ranges, the therapeutically effective amount can comprise from 10 mg to 225 mg fremanezumab, e.g., from 20 mg to 220 mg fremanezumab, from 30 mg to 215 mg fremanezumab, from 40 mg to 210 mg fremanezumab, from 50 mg to 205 mg fremanezumab, from 60 mg to 200 mg fremanezumab, from 70 mg to 190 mg fremanezumab, from 80 mg to 180 mg fremanezumab, from 90 mg to 170 mg fremanezumab, from 100 mg to 160 mg fremanezumab, from 10 mg to 30 mg fremanezumab, from 10 mg to 40 mg fremanezumab, from 10 mg to 45 mg fremanezumab, from 10 mg to 50 mg fremanezumab, from 10 mg to 75 mg fremanezumab, from 10 mg to 100 mg fremanezumab, from 10 mg to 115 mg fremanezumab, from 10 mg to 125 mg fremanezumab, from 15 mg to 90 mg fremanezumab, from 20 mg to 80 mg fremanezumab, from 25 mg to 75 mg fremanezumab, from 100 mg to 225 mg fremanezumab, from 110 mg to 220 mg fremanezumab, from 120 mg to 210 mg fremanezumab, from 125 mg to 175 mg fremanezumab, from 130 mg to 165 mg fremanezumab, from 50 mg to 150 mg fremanezumab, from 60 mg to 150 mg fremanezumab, from 75 mg to 150 mg fremanezumab, from 75 mg to 125 mg fremanezumab, from 150 mg to 225 mg fremanezumab, from 160 mg to 225 mg fremanezumab, from 175 mg to 225 mg fremanezumab, or from 200 mg to 225 mg fremanezumab. In some embodiments, the therapeutically effective amount comprises one or more additional active ingredients in addition to fremanezumab and the total active ingredients in the therapeutically effective amount comprise from 0.1 wt. % to 99.9 wt. % fremanezumab, e.g., up to 0.5 wt. % fremanezumab, up to 1 wt. % fremanezumab, up to 2 wt. % fremanezumab, up to 4 wt. % fremanezumab, up to 6 wt. % fremanezumab, up to 8 wt. % fremanezumab, up to 10 wt. % fremanezumab, up to 15 wt. % fremanezumab, up to 20 wt. % fremanezumab, up to 25 wt. % fremanezumab, up to 30 wt. % fremanezumab, up to 35 wt. % fremanezumab, up to 40 wt. % fremanezumab, up to 45 wt. % fremanezumab, up to 50 wt. % fremanezumab, up to 55 wt. % fremanezumab, up to 60 wt. % fremanezumab, up to 65 wt. % fremanezumab, up to 70 wt. % fremanezumab, up to 75 wt. % fremanezumab, up to 80 wt. % fremanezumab, up to 85 wt. % fremanezumab, up to 90 wt. % fremanezumab, up to 92 wt. % fremanezumab, up to 94 wt. % fremanezumab, up to 96 wt. % fremanezumab, up to 98 wt. % fremanezumab, up to 99 wt. % fremanezumab, or up to 99.5 wt. % fremanezumab. In some embodiments, the pharmaceutical composition comprising fremanezumab is administered to the subject 1 time/month.

In some embodiments, a therapeutically effective amount of a pharmaceutical composition described herein can comprise up to about 300 mg galcanezumab, e.g., up to 295 mg galcanezumab, up to 290 mg galcanezumab, up to 285 mg galcanezumab, up to 280 mg galcanezumab, up to 275 mg galcanezumab, up to 270 mg galcanezumab, up to 265 mg galcanezumab, up to 260 mg galcanezumab, up to 255 mg galcanezumab, up to 250 mg galcanezumab, up to 245 mg galcanezumab, up to 240 mg galcanezumab, up to 235 mg galcanezumab, up to 230 mg galcanezumab, up to 225 mg galcanezumab, up to 220 mg galcanezumab, up to 215 mg galcanezumab, up to 210 mg galcanezumab, up to 205 mg galcanezumab, up to 200 mg galcanezumab, up to 195 mg galcanezumab, up to 190 mg galcanezumab, up to 185 mg galcanezumab, up to 180 mg galcanezumab, up to 175 mg galcanezumab, up to 170 mg galcanezumab, up to 165 mg galcanezumab, up to 160 mg galcanezumab, up to 155 mg galcanezumab, up to 150 mg galcanezumab, up to 145 mg galcanezumab, up to 140 mg galcanezumab, up to 135 mg galcanezumab, up to 130 mg galcanezumab, up to 125 mg galcanezumab, up to 120 mg galcanezumab, up to 115 mg galcanezumab, up to 110 mg galcanezumab, up to 105 mg galcanezumab, up to 100 mg galcanezumab, up to 95 mg galcanezumab, up to 90 mg galcanezumab, up to 85 mg galcanezumab, up to 80 mg galcanezumab, up to 75 mg galcanezumab, up to 70 mg galcanezumab, up to 65 mg galcanezumab, up to 60 mg galcanezumab, up to 55 mg galcanezumab, up to 50 mg galcanezumab, up to 45 mg galcanezumab, up to 40 mg galcanezumab, up to 35 mg galcanezumab, up to 30 mg galcanezumab, up to 25 mg galcanezumab, up to 20 mg galcanezumab, up to 15 mg galcanezumab, or up to 10 mg galcanezumab. In terms of ranges, the therapeutically effective amount can comprise from 10 mg to 300 mg galcanezumab, e.g., from 20 mg to 290 mg galcanezumab, from 30 mg to 280 mg galcanezumab, from 40 mg to 270 mg galcanezumab, from 50 mg to 260 mg galcanezumab, from 60 mg to 250 mg galcanezumab, from 70 mg to 240 mg galcanezumab, from 80 mg to 230 mg galcanezumab, from 90 mg to 220 mg galcanezumab, from 100 mg to 210 mg galcanezumab, from 110 mg to 200 mg galcanezumab, from 120 mg to 190 mg galcanezumab, from 130 mg to 180 mg galcanezumab, from 140 mg to 170 mg galcanezumab, from 10 mg to 30 mg galcanezumab, from 10 mg to 40 mg galcanezumab, from 10 mg to 45 mg galcanezumab, from 10 mg to 50 mg galcanezumab, from 10 mg to 75 mg galcanezumab, from 10 mg to 100 mg galcanezumab, from 10 mg to 150 mg galcanezumab, from 100 mg to 150 mg galcanezumab, from 100 mg to 175 mg galcanezumab, from 100 mg to 200 mg galcanezumab, from 150 mg to 300 mg galcanezumab, from 150 mg to 275 mg galcanezumab, from 150 mg to 250 mg galcanezumab, from 150 mg to 225 mg galcanezumab, from 150 mg to 200 mg galcanezumab, from 200 mg to 300 mg galcanezumab, from 200 mg to 275 mg galcanezumab, or from 200 mg to 250 mg galcanezumab. In some embodiments, the therapeutically effective amount comprises one or more additional active ingredients in addition to galcanezumab and the total active ingredients in the therapeutically effective amount comprise from 0.1 wt. % to 99.9 wt. % galcanezumab, e.g., up to 0.5 wt. % galcanezumab, up to 1 wt. % galcanezumab, up to 2 wt. % galcanezumab, up to 4 wt. % galcanezumab, up to 6 wt. % galcanezumab, up to 8 wt. % galcanezumab, up to 10 wt. % galcanezumab, up to 15 wt. % galcanezumab, up to 20 wt. % galcanezumab, up to 25 wt. % galcanezumab, up to 30 wt. % galcanezumab, up to 35 wt. % galcanezumab, up to 40 wt. % galcanezumab, up to 45 wt. % galcanezumab, up to 50 wt. % galcanezumab, up to 55 wt. % galcanezumab, up to 60 wt. % galcanezumab, up to 65 wt. % galcanezumab, up to 70 wt. % galcanezumab, up to 75 wt. % galcanezumab, up to 80 wt. % galcanezumab, up to 85 wt. % galcanezumab, up to 90 wt. % galcanezumab, up to 92 wt. % galcanezumab, up to 94 wt. % galcanezumab, up to 96 wt. % galcanezumab, up to 98 wt. % galcanezumab, up to 99 wt. % galcanezumab, or up to 99.5 wt. % galcanezumab. In some embodiments, the pharmaceutical composition comprising galcanezumab is administered to the subject 1 time/month.

In some embodiments, a therapeutically effective amount of a pharmaceutical composition described herein can comprise up to about 0.6 mg clonidine, e.g., up to 0.58 mg clonidine, up to 0.56 mg clonidine, up to 0.54 mg clonidine, up to 0.52 mg clonidine, up to 0.50 mg clonidine, up to 0.48 mg clonidine, up to 0.46 mg clonidine, up to 0.44 mg clonidine, up to 0.42 mg clonidine, up to 0.40 mg clonidine, up to 0.38 mg clonidine, up to 0.36 mg clonidine, up to 0.34 mg clonidine, up to 0.32 mg clonidine, up to 0.30 mg clonidine, up to 0.28 mg clonidine, up to 0.26 mg clonidine, up to 0.24 mg clonidine, up to 0.22 mg clonidine, up to 0.20 mg clonidine, up to 0.18 mg clonidine, up to 0.16 mg clonidine, up to 0.14 mg clonidine, up to 0.12 mg clonidine, up to 0.10 mg clonidine, up to 0.08 mg clonidine, up to 0.06 mg clonidine, up to 0.04 mg clonidine, up to 0.02 mg clonidine, or up to 0.01 mg clonidine. In terms of ranges, the therapeutically effective amount can comprise from 0.01 mg to 0.60 mg clonidine, e.g., from 0.02 mg to 0.55 mg clonidine, from 0.05 mg to 0.50 mg clonidine, from 0.1 mg to 0.50 mg clonidine, from 0.20 mg to 0.40 mg clonidine, from 0.01 mg to 0.30 mg clonidine, from 0.01 mg to 0.20 mg clonidine, from 0.01 mg to 0.10 mg clonidine, from 0.10 mg to 0.40 mg clonidine, from 0.30 mg to 0.60 mg clonidine, from 0.40 mg to 0.60 mg clonidine, or from 0.50 mg to 0.60 mg clonidine. In some embodiments, the therapeutically effective amount comprises one or more additional active ingredients in addition to clonidine and the total active ingredients in the therapeutically effective amount comprise from 0.1 wt. % to 99.9 wt. % clonidine, e.g., up to 0.5 wt. % clonidine, up to 1 wt. % clonidine, up to 2 wt. % clonidine, up to 4 wt. % clonidine, up to 6 wt. % clonidine, up to 8 wt. % clonidine, up to 10 wt. % clonidine, up to 15 wt. % clonidine, up to 20 wt. % clonidine, up to 25 wt. % clonidine, up to 30 wt. % clonidine, up to 35 wt. % clonidine, up to 40 wt. % clonidine, up to 45 wt. % clonidine, up to 50 wt. % clonidine, up to 55 wt. % clonidine, up to 60 wt. % clonidine, up to 65 wt. % clonidine, up to 70 wt. % clonidine, up to 75 wt. % clonidine, up to 80 wt. % clonidine, up to 85 wt. % clonidine, up to 90 wt. % clonidine, up to 92 wt. % clonidine, up to 94 wt. % clonidine, up to 96 wt. % clonidine, up to 98 wt. % clonidine, up to 99 wt. % clonidine, or up to 99.5 wt. % clonidine. In some embodiments, the pharmaceutical composition comprising clonidine is administered to the subject 1 time/day.

In some embodiments, a therapeutically effective amount of a pharmaceutical composition described herein can comprise an SNRI at up to about 300 mg, e.g., up to 295 mg, up to 290 mg, up to 285 mg, up to 280 mg, up to 275 mg, up to 270 mg, up to 265 mg, up to 260 mg, up to 255 mg, up to 250 mg, up to 245 mg, up to 240 mg, up to 235 mg, up to 230 mg, up to 225 mg, up to 220 mg, up to 215 mg, up to 210 mg, up to 205 mg, up to 200 mg, up to 195 mg, up to 190 mg, up to 185 mg, up to 180 mg, up to 175 mg, up to 170 mg, up to 165 mg, up to 160 mg, up to 155 mg, up to 150 mg, up to 145 mg, up to 140 mg, up to 135 mg, up to 130 mg, up to 125 mg, up to 120 mg, up to 115 mg, up to 110 mg, up to 105 mg, up to 100 mg, up to 95 mg, up to 90 mg, up to 85 mg, up to 80 mg, up to 75 mg, up to 70 mg, up to 65 mg, up to 60 mg, up to 55 mg, up to 50 mg, up to 45 mg, up to 40 mg, up to 35 mg, up to 30 mg, up to 25 mg, up to 20 mg, up to 15 mg, up to 10 mg, up to 5 mg, up to 2 mg, up to 1 mg, up to 0.7 mg, up to 0.6 mg, up to 0.5 mg, up to 0.4 mg, up to 0.3 mg, up to 0.2 mg, or up to 0.1 mg. In terms of ranges, the therapeutically effective amount can comprise an SNRI from 0.1 mg to 300 mg, e.g., from 0.1 mg to 295 mg, from 0.5 mg to 295 mg, from 1 mg to 290 mg, from 5 mg to 290 mg, from 10 mg to 285 mg, from 20 mg to 285 mg, from 30 mg to 280 mg, from 40 mg to 270 mg, from 50 mg to 260 mg, from 60 mg to 250 mg, from 70 mg to 240 mg, from 80 mg to 230 mg, from 90 mg to 220 mg, from 100 mg to 210 mg, from 110 mg to 200 mg, from 120 mg to 190 mg, from 130 mg to 180 mg, from 140 mg to 170 mg, from 0.1 mg to 30 mg, from 0.1 mg to 40 mg, from 0.1 mg to 45 mg, from 0.1 mg to 50 mg, from 0.1 mg to 75 mg, from 0.1 mg to 100 mg, from 0.1 mg to 150 mg, from 100 mg to 150 mg, from 100 mg to 175 mg, from 100 mg to 200 mg, from 150 mg to 300 mg, from 150 mg to 275 mg, from 150 mg to 250 mg, from 150 mg to 225 mg, from 150 mg to 200 mg, from 200 mg to 300 mg, from 200 mg to 275 mg, or from 200 mg to 250 mg. In some embodiments, the therapeutically effective amount comprises one or more additional active ingredients in addition to an SNRI, and the total active ingredients in the therapeutically effective amount comprise from 0.1 wt. % to 99.9 wt. % SNRI, e.g., up to 0.5 wt. % SNRI, up to 1 wt. % SNRI, up to 2 wt. % SNRI, up to 4 wt. % SNRI, up to 6 wt. % SNRI, up to 8 wt. % SNRI, up to 10 wt. % SNRI, up to 15 wt. % SNRI, up to 20 wt. % SNRI, up to 25 wt. % SNRI, up to 30 wt. % SNRI, up to 35 wt. % SNRI, up to 40 wt. % SNRI, up to 45 wt. % SNRI, up to 50 wt. % SNRI, up to 55 wt. % SNRI, up to 60 wt. % SNRI, up to 65 wt. % SNRI, up to 70 wt. % SNRI, up to 75 wt. % SNRI, up to 80 wt. % SNRI, up to 85 wt. % SNRI, up to 90 wt. % SNRI, up to 92 wt. % SNRI, up to 94 wt. % SNRI, up to 96 wt. % SNRI, up to 98 wt. % SNRI, up to 99 wt. % SNRI, or up to 99.5 wt. % SNRI.

In some embodiments, a therapeutically effective amount of a pharmaceutical composition described herein can comprise a CGRP receptor antagonist at up to about 300 mg, e.g., up to 295 mg, up to 290 mg, up to 285 mg, up to 280 mg, up to 275 mg, up to 270 mg, up to 265 mg, up to 260 mg, up to 255 mg, up to 250 mg, up to 245 mg, up to 240 mg, up to 235 mg, up to 230 mg, up to 225 mg, up to 220 mg, up to 215 mg, up to 210 mg, up to 205 mg, up to 200 mg, up to 195 mg, up to 190 mg, up to 185 mg, up to 180 mg, up to 175 mg, up to 170 mg, up to 165 mg, up to 160 mg, up to 155 mg, up to 150 mg, up to 145 mg, up to 140 mg, up to 135 mg, up to 130 mg, up to 125 mg, up to 120 mg, up to 115 mg, up to 110 mg, up to 105 mg, up to 100 mg, up to 95 mg, up to 90 mg, up to 85 mg, up to 80 mg, up to 75 mg, up to 70 mg, up to 65 mg, up to 60 mg, up to 55 mg, up to 50 mg, up to 45 mg, up to 40 mg, up to 35 mg, up to 30 mg, up to 25 mg, up to 20 mg, up to 15 mg, up to 10 mg, up to 5 mg, up to 2 mg, up to 1 mg, up to 0.7 mg, up to 0.6 mg, up to 0.5 mg, up to 0.4 mg, up to 0.3 mg, up to 0.2 mg, or up to 0.1 mg. In terms of ranges, the therapeutically effective amount can comprise a CGRP receptor antagonist from 0.1 mg to 300 mg, e.g., from 0.1 mg to 295 mg, from 0.5 mg to 295 mg, from 1 mg to 290 mg, from 5 mg to 290 mg, from 10 mg to 285 mg, from 20 mg to 285 mg, from 30 mg to 280 mg, from 40 mg to 270 mg, from 50 mg to 260 mg, from 60 mg to 250 mg, from 70 mg to 240 mg, from 80 mg to 230 mg, from 90 mg to 220 mg, from 100 mg to 210 mg, from 110 mg to 200 mg, from 120 mg to 190 mg, from 130 mg to 180 mg, from 140 mg to 170 mg, from 0.1 mg to 30 mg, from 0.1 mg to 40 mg, from 0.1 mg to 45 mg, from 0.1 mg to 50 mg, from 0.1 mg to 75 mg, from 0.1 mg to 100 mg, from 0.1 mg to 150 mg, from 100 mg to 150 mg, from 100 mg to 175 mg, from 100 mg to 200 mg, from 150 mg to 300 mg, from 150 mg to 275 mg, from 150 mg to 250 mg, from 150 mg to 225 mg, from 150 mg to 200 mg, from 200 mg to 300 mg, from 200 mg to 275 mg, or from 200 mg to 250 mg. In some embodiments, the therapeutically effective amount comprises one or more additional active ingredients in addition to a CGRP receptor antagonist, and the total active ingredients in the therapeutically effective amount comprise from 0.1 wt. % to 99.9 wt. % CGRP receptor antagonist, e.g., up to 0.5 wt. % CGRP receptor antagonist, up to 1 wt. % CGRP receptor antagonist, up to 2 wt. % CGRP receptor antagonist, up to 4 wt. % CGRP receptor antagonist, up to 6 wt. % CGRP receptor antagonist, up to 8 wt. % CGRP receptor antagonist, up to 10 wt. % CGRP receptor antagonist, up to 15 wt. % CGRP receptor antagonist, up to 20 wt. % CGRP receptor antagonist, up to 25 wt. % CGRP receptor antagonist, up to 30 wt. % CGRP receptor antagonist, up to 35 wt. % CGRP receptor antagonist, up to 40 wt. % CGRP receptor antagonist, up to 45 wt. % CGRP receptor antagonist, up to 50 wt. % CGRP receptor antagonist, up to 55 wt. % CGRP receptor antagonist, up to 60 wt. % CGRP receptor antagonist, up to 65 wt. % CGRP receptor antagonist, up to 70 wt. % CGRP receptor antagonist, up to 75 wt. % CGRP receptor antagonist, up to 80 wt. % CGRP receptor antagonist, up to 85 wt. % CGRP receptor antagonist, up to 90 wt. % CGRP receptor antagonist, up to 92 wt. % CGRP receptor antagonist, up to 94 wt. % CGRP receptor antagonist, up to 96 wt. % CGRP receptor antagonist, up to 98 wt. % CGRP receptor antagonist, up to 99 wt. % CGRP receptor antagonist, or up to 99.5 wt. % CGRP receptor antagonist.

In some embodiments, a therapeutically effective amount of a pharmaceutical composition described herein can comprise an anti-CGRP monoclonal antibody at up to about 300 mg, e.g., up to 295 mg, up to 290 mg, up to 285 mg, up to 280 mg, up to 275 mg, up to 270 mg, up to 265 mg, up to 260 mg, up to 255 mg, up to 250 mg, up to 245 mg, up to 240 mg, up to 235 mg, up to 230 mg, up to 225 mg, up to 220 mg, up to 215 mg, up to 210 mg, up to 205 mg, up to 200 mg, up to 195 mg, up to 190 mg, up to 185 mg, up to 180 mg, up to 175 mg, up to 170 mg, up to 165 mg, up to 160 mg, up to 155 mg, up to 150 mg, up to 145 mg, up to 140 mg, up to 135 mg, up to 130 mg, up to 125 mg, up to 120 mg, up to 115 mg, up to 110 mg, up to 105 mg, up to 100 mg, up to 95 mg, up to 90 mg, up to 85 mg, up to 80 mg, up to 75 mg, up to 70 mg, up to 65 mg, up to 60 mg, up to 55 mg, up to 50 mg, up to 45 mg, up to 40 mg, up to 35 mg, up to 30 mg, up to 25 mg, up to 20 mg, up to 15 mg, up to 10 mg, up to 5 mg, up to 2 mg, up to 1 mg, up to 0.7 mg, up to 0.6 mg, up to 0.5 mg, up to 0.4 mg, up to 0.3 mg, up to 0.2 mg, or up to 0.1 mg. In terms of ranges, the therapeutically effective amount can comprise an anti-CGRP monoclonal antibody from 0.1 mg to 300 mg, e.g., from 0.1 mg to 295 mg, from 0.5 mg to 295 mg, from 1 mg to 290 mg, from 5 mg to 290 mg, from 10 mg to 285 mg, from 20 mg to 285 mg, from 30 mg to 280 mg, from 40 mg to 270 mg, from 50 mg to 260 mg, from 60 mg to 250 mg, from 70 mg to 240 mg, from 80 mg to 230 mg, from 90 mg to 220 mg, from 100 mg to 210 mg, from 110 mg to 200 mg, from 120 mg to 190 mg, from 130 mg to 180 mg, from 140 mg to 170 mg, from 0.1 mg to 30 mg, from 0.1 mg to 40 mg, from 0.1 mg to 45 mg, from 0.1 mg to 50 mg, from 0.1 mg to 75 mg, from 0.1 mg to 100 mg, from 0.1 mg to 150 mg, from 100 mg to 150 mg, from 100 mg to 175 mg, from 100 mg to 200 mg, from 150 mg to 300 mg, from 150 mg to 275 mg, from 150 mg to 250 mg, from 150 mg to 225 mg, from 150 mg to 200 mg, from 200 mg to 300 mg, from 200 mg to 275 mg, or from 200 mg to 250 mg. In some embodiments, the therapeutically effective amount comprises one or more additional active ingredients in addition to an anti-CGRP monoclonal antibody, and the total active ingredients in the therapeutically effective amount comprise from 0.1 wt. % to 99.9 wt. % anti-CGRP monoclonal antibody, e.g., up to 0.5 wt. % anti-CGRP monoclonal antibody, up to 1 wt. % anti-CGRP monoclonal antibody, up to 2 wt. % anti-CGRP monoclonal antibody, up to 4 wt. % anti-CGRP monoclonal antibody, up to 6 wt. % anti-CGRP monoclonal antibody, up to 8 wt. % anti-CGRP monoclonal antibody, up to 10 wt. % anti-CGRP monoclonal antibody, up to 15 wt. % anti-CGRP monoclonal antibody, up to 20 wt. % anti-CGRP monoclonal antibody, up to 25 wt. % anti-CGRP monoclonal antibody, up to 30 wt. % anti-CGRP monoclonal antibody, up to 35 wt. % anti-CGRP monoclonal antibody, up to 40 wt. % anti-CGRP monoclonal antibody, up to 45 wt. % anti-CGRP monoclonal antibody, up to 50 wt. % anti-CGRP monoclonal antibody, up to 55 wt. % anti-CGRP monoclonal antibody, up to 60 wt. % anti-CGRP monoclonal antibody, up to 65 wt. % anti-CGRP monoclonal antibody, up to 70 wt. % anti-CGRP monoclonal antibody, up to 75 wt. % anti-CGRP monoclonal antibody, up to 80 wt. % anti-CGRP monoclonal antibody, up to 85 wt. % anti-CGRP monoclonal antibody, up to 90 wt. % anti-CGRP monoclonal antibody, up to 92 wt. % anti-CGRP monoclonal antibody, up to 94 wt. % anti-CGRP monoclonal antibody, up to 96 wt. % anti-CGRP monoclonal antibody, up to 98 wt. % anti-CGRP monoclonal antibody, up to 99 wt. % anti-CGRP monoclonal antibody, or up to 99.5 wt. % anti-CGRP monoclonal antibody.

In some embodiments, a therapeutically effective amount of a pharmaceutical composition described herein can comprise an alpha-2C agonist at up to about 300 mg, e.g., up to 295 mg, up to 290 mg, up to 285 mg, up to 280 mg, up to 275 mg, up to 270 mg, up to 265 mg, up to 260 mg, up to 255 mg, up to 250 mg, up to 245 mg, up to 240 mg, up to 235 mg, up to 230 mg, up to 225 mg, up to 220 mg, up to 215 mg, up to 210 mg, up to 205 mg, up to 200 mg, up to 195 mg, up to 190 mg, up to 185 mg, up to 180 mg, up to 175 mg, up to 170 mg, up to 165 mg, up to 160 mg, up to 155 mg, up to 150 mg, up to 145 mg, up to 140 mg, up to 135 mg, up to 130 mg, up to 125 mg, up to 120 mg, up to 115 mg, up to 110 mg, up to 105 mg, up to 100 mg, up to 95 mg, up to 90 mg, up to 85 mg, up to 80 mg, up to 75 mg, up to 70 mg, up to 65 mg, up to 60 mg, up to 55 mg, up to 50 mg, up to 45 mg, up to 40 mg, up to 35 mg, up to 30 mg, up to 25 mg, up to 20 mg, up to 15 mg, up to 10 mg, up to 5 mg, up to 2 mg, up to 1 mg, up to 0.7 mg, up to 0.6 mg, up to 0.5 mg, up to 0.4 mg, up to 0.3 mg, up to 0.2 mg, or up to 0.1 mg. In terms of ranges, the therapeutically effective amount can comprise an alpha-2C agonist from 0.1 mg to 300 mg, e.g., from 0.1 mg to 295 mg, from 0.5 mg to 295 mg, from 1 mg to 290 mg, from 5 mg to 290 mg, from 10 mg to 285 mg, from 20 mg to 285 mg, from 30 mg to 280 mg, from 40 mg to 270 mg, from 50 mg to 260 mg, from 60 mg to 250 mg, from 70 mg to 240 mg, from 80 mg to 230 mg, from 90 mg to 220 mg, from 100 mg to 210 mg, from 110 mg to 200 mg, from 120 mg to 190 mg, from 130 mg to 180 mg, from 140 mg to 170 mg, from 0.1 mg to 30 mg, from 0.1 mg to 40 mg, from 0.1 mg to 45 mg, from 0.1 mg to 50 mg, from 0.1 mg to 75 mg, from 0.1 mg to 100 mg, from 0.1 mg to 150 mg, from 100 mg to 150 mg, from 100 mg to 175 mg, from 100 mg to 200 mg, from 150 mg to 300 mg, from 150 mg to 275 mg, from 150 mg to 250 mg, from 150 mg to 225 mg, from 150 mg to 200 mg, from 200 mg to 300 mg, from 200 mg to 275 mg, or from 200 mg to 250 mg. In some embodiments, the therapeutically effective amount comprises one or more additional active ingredients in addition to an alpha-2C agonist, and the total active ingredients in the therapeutically effective amount comprise from 0.1 wt. % to 99.9 wt. % alpha-2C agonist, e.g., up to 0.5 wt. % alpha-2C agonist, up to 1 wt. % alpha-2C agonist, up to 2 wt. % alpha-2C agonist, up to 4 wt. % alpha-2C agonist, up to 6 wt. % alpha-2C agonist, up to 8 wt. % alpha-2C agonist, up to 10 wt. % alpha-2C agonist, up to 15 wt. % alpha-2C agonist, up to 20 wt. % alpha-2C agonist, up to 25 wt. % alpha-2C agonist, up to 30 wt. % alpha-2C agonist, up to 35 wt. % alpha-2C agonist, up to 40 wt. % alpha-2C agonist, up to 45 wt. % alpha-2C agonist, up to 50 wt. % alpha-2C agonist, up to 55 wt. % alpha-2C agonist, up to 60 wt. % alpha-2C agonist, up to 65 wt. % alpha-2C agonist, up to 70 wt. % alpha-2C agonist, up to 75 wt. % alpha-2C agonist, up to 80 wt. % alpha-2C agonist, up to 85 wt. % alpha-2C agonist, up to 90 wt. % alpha-2C agonist, up to 92 wt. % alpha-2C agonist, up to 94 wt. % alpha-2C agonist, up to 96 wt. % alpha-2C agonist, up to 98 wt. % alpha-2C agonist, up to 99 wt. % alpha-2C agonist, or up to 99.5 wt. % alpha-2C agonist.

In some embodiments, a therapeutically effective amount of a pharmaceutical composition disclosed herein comprises a combination of at least two components selected from the categories consisting of: 1) a serotonin-norepinephrine reuptake inhibitor (SNRI); 2) a calcitonin gene-related peptide (CGRP) receptor antagonist; 3) an anti-CGRP monoclonal antibody; and 4) an alpha-2C agonist. The at least two components may be present at any ratio relative to each other.

In some embodiments, a therapeutically effective amount of a pharmaceutical composition disclosed herein comprises an SNRI and a CGRP receptor antagonist, wherein the ratio of SNRI wt. % to CGRP receptor antagonist wt. % can be from about 10:1 to about 1:10, e.g., about 10:1 to about 1.5:1; about 10:1 to about 2:1; about 9:1 to about 1.5:1; about 9:1 to about 2:1; about 8:1 to about 1.5:1; about 8:1 to about 2:1; about 7:1 to about 2:1; about 5:1 to about 2:1; about 4:1 to about 2:1; or about 3:1 to about 2:1; about 1:2 to about 1:3; about 1:2 to about 1:4; about 1:2 to about 1:5; about 1:2 to about 1:7; about 1:8 to about 1:2; about 1:1.5 to about 1:8; about 1:2 to about 1:9; about 1:1.5 to about 1:9; about 1:2 to about 1:10; or about 1:1.5 to about 1:10. In some embodiments, the therapeutically effective amount comprises an SNRI and an anti-CGRP monoclonal antibody, wherein the ratio of SNRI wt. % to anti-CGRP monoclonal antibody wt. % can be from about 10:1 to about 1:10, e.g., about 10:1 to about 1.5:1; about 10:1 to about 2:1; about 9:1 to about 1.5:1; about 9:1 to about 2:1; about 8:1 to about 1.5:1; about 8:1 to about 2:1; about 7:1 to about 2:1; about 5:1 to about 2:1; about 4:1 to about 2:1; or about 3:1 to about 2:1; about 1:2 to about 1:3; about 1:2 to about 1:4; about 1:2 to about 1:5; about 1:2 to about 1:7; about 1:8 to about 1:2; about 1:1.5 to about 1:8; about 1:2 to about 1:9; about 1:1.5 to about 1:9; about 1:2 to about 1:10; or about 1:1.5 to about 1:10. In some embodiments, the therapeutically effective amount comprises an SNRI and an alpha-2C agonist, wherein the ratio of SNRI wt. % to alpha-2C agonist wt. % can be from about 10:1 to about 1:10, e.g., about 10:1 to about 1.5:1; about 10:1 to about 2:1; about 9:1 to about 1.5:1; about 9:1 to about 2:1; about 8:1 to about 1.5:1; about 8:1 to about 2:1; about 7:1 to about 2:1; about 5:1 to about 2:1; about 4:1 to about 2:1; or about 3:1 to about 2:1; about 1:2 to about 1:3; about 1:2 to about 1:4; about 1:2 to about 1:5; about 1:2 to about 1:7; about 1:8 to about 1:2; about 1:1.5 to about 1:8; about 1:2 to about 1:9; about 1:1.5 to about 1:9; about 1:2 to about 1:10; or about 1:1.5 to about 1:10.

In some embodiments, the therapeutically effective amount comprises a CGRP receptor antagonist and an anti-CGRP monoclonal antibody, wherein the ratio of CGRP receptor antagonist wt. % to anti-CGRP monoclonal antibody wt. % can be from about 10:1 to about 1:10, e.g., about 10:1 to about 1.5:1; about 10:1 to about 2:1; about 9:1 to about 1.5:1; about 9:1 to about 2:1; about 8:1 to about 1.5:1; about 8:1 to about 2:1; about 7:1 to about 2:1; about 5:1 to about 2:1; about 4:1 to about 2:1; or about 3:1 to about 2:1; about 1:2 to about 1:3; about 1:2 to about 1:4; about 1:2 to about 1:5; about 1:2 to about 1:7; about 1:8 to about 1:2; about 1:1.5 to about 1:8; about 1:2 to about 1:9; about 1:1.5 to about 1:9; about 1:2 to about 1:10; or about 1:1.5 to about 1:10. In some embodiments, the therapeutically effective amount comprises a CGRP receptor antagonist and an alpha-2C agonist, wherein the ratio of CGRP receptor antagonist wt. % to alpha-2C agonist wt. % can be from about 10:1 to about 1:10, e.g., about 10:1 to about 1.5:1; about 10:1 to about 2:1; about 9:1 to about 1.5:1; about 9:1 to about 2:1; about 8:1 to about 1.5:1; about 8:1 to about 2:1; about 7:1 to about 2:1; about 5:1 to about 2:1; about 4:1 to about 2:1; or about 3:1 to about 2:1; about 1:2 to about 1:3; about 1:2 to about 1:4; about 1:2 to about 1:5; about 1:2 to about 1:7; about 1:8 to about 1:2; about 1:1.5 to about 1:8; about 1:2 to about 1:9; about 1:1.5 to about 1:9; about 1:2 to about 1:10; or about 1:1.5 to about 1:10. In some embodiments, the therapeutically effective amount comprises an anti-CGRP monoclonal antibody and an alpha-2C agonist, wherein the ratio of anti-CGRP monoclonal antibody wt. % to alpha-2C agonist wt. % can be from about 10:1 to about 1:10, e.g., about 10:1 to about 1.5:1; about 10:1 to about 2:1; about 9:1 to about 1.5:1; about 9:1 to about 2:1; about 8:1 to about 1.5:1; about 8:1 to about 2:1; about 7:1 to about 2:1; about 5:1 to about 2:1; about 4:1 to about 2:1; or about 3:1 to about 2:1; about 1:2 to about 1:3; about 1:2 to about 1:4; about 1:2 to about 1:5; about 1:2 to about 1:7; about 1:8 to about 1:2; about 1:1.5 to about 1:8; about 1:2 to about 1:9; about 1:1.5 to about 1:9; about 1:2 to about 1:10; or about 1:1.5 to about 1:10.

Suitable human doses of any of the pharmaceutical compositions described herein can further be evaluated in, e.g., Phase I dose escalation studies. See, e.g., van Gurp et al. (2008) Am J Transplantation 8 (8): 1711-1718; Hanouska et al. (2007) Clin Cancer Res 13 (2, part 1): 523-531; and Hetherington et al. (2006) Antimicrobial Agents and Chemotherapy 50 (10): 3499-3500.

In some embodiments, suitable human doses of any of the pharmaceutical compositions described herein can be evaluated according to a trial study. In some embodiments, such a study may involve recruiting a cohort of fibromyalgia or post-traumatic stress disorder patients. In some embodiments, patient information (e.g., demographic data, vital signs, medication history, baseline pain ratings, etc.) may be collected. In some embodiments, current medications other than the pharmaceutical composition being tested may be either washed out prior to the trial or held constand during the trial. In some embodiments, the trial may run for 8-20 weeks (e.g., 10 weeks, 12 weeks, 14 weeks, 16 weeks, 18 weeks). In some embodiments, outcomes of interest (e.g., pain reduction, sleep quality and/or quantity, side effect profile, etc.) may be collected at regular intervals (e.g., daily, weekly, monthly, etc.).

In some embodiments, during the first period of a 12-week trial study (e.g., first 4 weeks), a first component of a pharmaceutical composition as described herein may be administered via any of the administration routes described herein (e.g., oral route or injection). In some embodiments, during the second period (e.g., weeks 5-8), a second component of the pharmaceutical composition may be added to the first component and administered via any of the administration routes described herein (e.g., oral route or injection). In some embodiments, during the third period (e.g., weeks 9-12), a third component of the pharmaceutical composition may be added to the first and second components and administered via any of the administration routes described herein (e.g., oral route or injection). In some embodiments, a trial study may include a control group receiving standard of care treatment. In some embodiments, a trial study may be designed in a cross over fashion, i.e., where the patient group initially receiving a pharmaceutical composition described herein will cross over to receiving standard of care treatment and a control patient group initially receiving standard of care treatment may cross over to receive a pharmaceutical composition as described herein. In some embodiments, a trial study may be designed as an “N of one” trial, i.e., each enrolled subject may function as their own control by receiving standard of care treatment initially followed by receiving a pharmaceutical composition as described herein.

In some embodiments, a pharmaceutical composition described herein can be administered to a subject as a monotherapy. Alternatively, the pharmaceutical composition can be administered in conjunction with other therapies for chronic pain conditions (combination therapy). For example, the composition can be administered to a subject at the same time, prior to, or after, a second therapy. In some embodiments, the pharmaceutical composition and the one or more additional active agents are administered at the same time. Optionally, the pharmaceutical composition described herein can be administered first in time and the one or more additional active agents are administered second in time. In some embodiments, the one or more additional active agents are administered first in time and the pharmaceutical composition described herein is administered second in time. Optionally, the pharmaceutical composition and the one or more additional agents can be administered simultaneously in the same or different routes. For example, a composition comprising the active ingredients described herein optionally contains one or more additional agents. In certain embodiments, the additional agents may include, for example, pregabalin (Lyrica), tramadol (Ultram), or cyclobenzaprine (Flexeril).

A pharmaceutical composition described herein can replace or augment a previously or currently administered therapy. For example, upon treating with a pharmaceutical composition comprising a combination of the active ingredients as described herein, administration of the one or more additional active agents can cease or diminish, e.g., be administered at lower levels or dosages. In some embodiments, administration of the previous therapy can be maintained.

Monitoring a subject (e.g., a human patient) for an improvement of a chronic pain disorder, as defined herein, means evaluating the subject for a change in a disorder parameter, e.g., a reduction in one or more symptoms of the chronic pain disorder exhibited by the subject. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality, e.g., adding or dropping any of the treatments for a chronic pain disorder described herein. In some embodiments, the evaluation can include evaluating whether a therapeutic composition comprising a different combination of active ingredients should be administered.

The present disclosure will be better understood in view of the following non-limiting examples.

### EXAMPLES

### Example 1

A patient with diagnosed Fibromyalgia who has failed prior treatments (due to inefficacy or side effects) or does not achieve the desired relief from the current medical regimen or has never been successfully treated with medications will be started on Duloxetine 20 mg QD or BID for 4 weeks and the outcomes of interest and potential unwanted effects will be documented. Then a second component (i.e. a CGRP antagonist like Rimegepant, one dose (75 mg or less) under the tongue every other day) will be added for the following 4 weeks and the outcomes of interest and unwanted effects will be documented. Then a third component (i.e. a CGRP receptor antagonist like Erenumab, 70 mg injected once subcutaneously) or an alpha2C agonist (i.e. clonidine 0.1 mg PO daily) may be added. Again, the outcomes of interest and unwanted effects will be documented.

### Illustrations

Illustration 1: a pharmaceutical composition comprising at least two active ingredients selected from: 1) a serotonin-norepinephrine reuptake inhibitor (SNRI); 2) a calcitonin gene-related peptide (CGRP) receptor antagonist; 3) an anti-CGRP monoclonal antibody; and 4) an alpha-2C agonist.

Illustration 2: the composition of Illustration 1, wherein the SNRI compound comprises duloxetine, milnacipran, or a combination thereof.

Illustration 3: the composition of Illustration 1 or 2, wherein the CGRP receptor antagonist comprises rimegepant, fremanezumab, atogepant, erenumab, or a combination thereof.

Illustration 4: the composition of any of illustrations 1 to 3, wherein the anti-CGRP monoclonal antibody comprises eptinezumab, fremanezumab, galcanezumab, or a combination thereof.

Illustration 5: the composition of any of illustrations 1 to 4, wherein the alpha-2C agonist comprises clonidine.

Illustration 6: the composition of any of illustrations 1 to 5, wherein the composition comprises an SNRI and CGRP receptor antagonist.

Illustration 7: the composition of any of illustrations 1 to 6, wherein the composition comprises an SNRI and an anti-CGRP monoclonal antibody.

Illustration 8: the composition of any one of illustrations 1 to 7, wherein the composition comprises an SNRI, a CGRP receptor antagonist, and an anti-CGRP monoclonal antibody.

Illustration 9: the composition of any one of illustrations 6 to 8, wherein the SNRI comprises duloxetine.

Illustration 10: the composition of any preceding illustration, wherein the composition is administrable orally, intravenously, or intramuscularly.

Illustration 10: a unit dose of the composition of any preceding illustration.

Illustration 12: the unit dose of Illustration 11, wherein at least one composition component is present at a lower dose than the standard dose for said component.

Illustration 13: the unit dose of Illustration 12, wherein the composition component is present at less than about 75% of the standard dose.

Illustration 14: the unit dose of Illustration 12 or 13, wherein the composition component is present at less than about 50% of the standard dose.

Illustration 15: the unit dose of any one of illustrations 12 to 14, wherein the composition component is present at less than about 35% of the standard dose.

Illustration 16: a method of treating a subject with fibromyalgia, post-traumatic stress disorder, myalgic encephalomyelitis/chronic fatigue syndrome, irritable bowel syndrome, post-viral syndromes, long COVID, post-war syndromes, Post-Gulf War, environmentally implicated syndromes, chemical hypersensitivity, sick-building syndrome, or combinations thereof, comprising administering to the subject a therapeutically effective amount of the composition of any one of illustrations 1 to 9.

Illustration 17: a method of treating a subject with fibromyalgia, post-traumatic stress disorder, myalgic encephalomyelitis/chronic fatigue syndrome, or combinations thereof, comprising administering to the subject a therapeutically effective amount of the composition of any one of illustrations 1 to 9.

Illustration 18: a method of treating a subject with fibromyalgia, comprising administering to the subject a therapeutically effective amount of the composition of any one of illustrations 1 to 9.

Illustration 19: a method of treating a subject displaying the symptoms of diffuse widespread pain, fatigue, and brain fog, comprising administering to the subject a therapeutically effective amount of the composition of any one of illustrations 1 to 9.

Illustration 20: the method of any one of illustrations 16 to 19, wherein the composition is administered to the subject orally, intravenously, or intramuscularly.

Illustration 21: the method of any one of illustrations 16 to 20, wherein the therapeutically effective amount comprises a lower dose of at least one composition component than the standard dose for said component.

Illustration 22: the method of Illustration 21, wherein the composition component is present at less than about 75% of the standard dose.

Illustration 23: the method of Illustration 21 or 22, wherein the composition component is present at less than about 50% of the standard dose.

Illustration 24: the method of any one of illustrations 21 to 23, wherein the composition component is present at less than about 35% of the standard dose.

Illustration 25: the method of any one of illustrations 21 to 24, wherein the lower dose prevents undesirable side effects.

While the invention has been described in detail, modifications within the spirit and scope of the invention will be readily apparent to those of skill in the art. It should be understood that aspects of the invention and portions of various embodiments and various features recited above and/or in the appended claims may be combined or interchanged either in whole or in part. In the foregoing descriptions of the various embodiments, those embodiments which refer to another embodiment may be appropriately combined with other embodiments as will be appreciated by one of ordinary skill in the art. Furthermore, those of ordinary skill in the art will appreciate that the foregoing description is by way of example only, and is not intended to limit the invention.

1. A pharmaceutical composition comprising at least two active ingredients selected from:
1. a serotonin-norepinephrine reuptake inhibitor (SNRI);
2. a calcitonin gene-related peptide (CGRP) receptor antagonist;
3. an anti-CGRP monoclonal antibody; and
4. an alpha-2C agonist.

2. The composition of claim 1, wherein the SNRI compound comprises duloxetine, milnacipran, or a combination thereof.

3. The composition of claim 1, wherein the CGRP receptor antagonist comprises rimegepant, fremanezumab, atogepant, erenumab, or a combination thereof.

4. The composition of claim 1, wherein the anti-CGRP monoclonal antibody comprises eptinezumab, fremanezumab, galcanezumab, or a combination thereof.

5. The composition of claim 1, wherein the alpha-2C agonist comprises clonidine.

6. The composition of claim 1, wherein the composition comprises an SNRI and CGRP receptor antagonist.

7. The composition of claim 1, wherein the composition comprises an SNRI and an anti-CGRP monoclonal antibody.

8. The composition of claim 1, wherein the composition comprises an SNRI, a CGRP receptor antagonist, and an anti-CGRP monoclonal antibody.

9. The composition of claim 6, wherein the SNRI comprises duloxetine.

10. The composition of claim 1, wherein the composition is administrable orally, intravenously, or intramuscularly.

11. A unit dose of the composition of claim 1.

12. The unit dose of claim 11, wherein at least one composition component is present at a lower dose than the standard dose for said component.

13. The unit dose of claim 12, wherein the composition component is present at less than about 75% of the standard dose.

14. (canceled)

15. (canceled)

16. A method of treating a subject with fibromyalgia, post-traumatic stress disorder, myalgic encephalomyelitis/chronic fatigue syndrome, irritable bowel syndrome, post-viral syndromes, long COVID, post-war syndromes, Post-Gulf War, environmentally implicated syndromes, chemical hypersensitivity, sick-building syndrome, or combinations thereof, comprising administering to the subject a therapeutically effective amount of the composition of claim 1.

17. A method of treating a subject with fibromyalgia, post-traumatic stress disorder, myalgic encephalomyelitis/chronic fatigue syndrome, or combinations thereof, comprising administering to the subject a therapeutically effective amount of the composition of claim 1.

18. A method of treating a subject with fibromyalgia, comprising administering to the subject a therapeutically effective amount of the composition of claim 1.

19. A method of treating a subject displaying the symptoms of diffuse widespread pain, fatigue, and brain fog, comprising administering to the subject a therapeutically effective amount of the composition of claim 1.

20. The method of claim 16, wherein the composition is administered to the subject orally, intravenously, or intramuscularly.

21. The method of claim 16, wherein the therapeutically effective amount comprises a lower dose of at least one composition component than the standard dose for said component.

22. The method of claim 21, wherein the composition component is present at less than about 75% of the standard dose.

23. (canceled)

24. (canceled)

25. The method of claim 21, wherein the lower dose prevents undesirable side effects.
